(19) United States
(12) Patent Application Publication
(10) Pub. No.: US 2025/0134526 A1
(43) Pub. Date: May 1, 2025

(54) INTRAVASCULAR DEVICE

(71) Applicant: LuSeed Vascular Ltd., Tel-Aviv (IL)

(72) Inventors: Amir ARTHUR, Rishon LeTsion (IL); Nitzan HIRSH, Tel Aviv (IL); Moshe MILLER, ModiIn Makabim-ReUt (IL); Shraga CINER, Herzliya (IL); Shlomo KANTOR, Bikat HaYarden (IL)

(73) Assignee: LuSeed Vascular Ltd., Tel-Aviv (IL)

(21) Appl. No.: 19/004,580

(22) Filed: Dec. 30, 2024

Related U.S. Application Data
(63) Continuation of application No. 18/219,809, filed on Jul. 10, 2023.
Application No. 18/219,809 is a continuation of application No. PCT/IL2022/050032, filed on Jan. 10, 2022.
(60) Provisional application No. 63/240,939, filed on Sep. 5, 2021.
(60) Provisional application No. 63/135,629, filed on Jan. 10, 2021.

(51) Int. Cl. A61B 17/12

(52) U.S. Cl. CPC A61B 17/12113; A61B 17/12172; A61B 2017/12063; A61B 2017/12068

(57) ABSTRACT

An intravascular device comprising: an expandable body having an expanded configuration and a contracted configuration, said expandable body in said expanded configuration having a convex curved shape having a base and walls; and a connector; wherein said connector is attached to said expandable body and recessed within said convex curved shape.

### RELATED APPLICATIONS

This application is a Continuation of U.S. patent application Ser. No. 18/219,809 filed on Jul. 10, 2023, which is a Continuation of PCT Patent Application No. PCT/IL2022/050032 having International filing date of Jan. 10, 2022, which claims the benefit of priority under 35 USC § 119(e) of U.S. Provisional Patent Application Nos. 63/240,939 filed on Sep. 5, 2021 and 63/135,629 filed on Jan. 10, 2021. The contents of the above applications are all incorporated by reference as if fully set forth herein in their entirety.

### FIELD AND BACKGROUND OF THE INVENTION

The present invention, in some embodiments thereof, relates to treatment of intravascular abnormalities and, more particularly, but not exclusively, to treatment of aneurysm using an expandable intravascular device.

U.S. Patent Application No. 2011/0144669 discloses “An implant for treating brain aneurysms, especially terminal aneurysms, comprises a neck cover and elongate shaft removably secured to an embolic delivery catheter. As such, the shaft aids in directing and placing the cover at the aneurysm neck, protecting the delivery catheter from adhesion with the embolic material, and securing the cover in place with connection or adhesion of the shaft to the embolic material delivered through the catheter. The implant can be anchored at the aneurysm either by interface and/or adhesion of the shaft or shaft and cover with the resident embolic materials.”

U.S. Patent Application No. 2012/0330341 discloses “Embolic implants, methods of manufacture and delivery are disclosed. The subject implants are especially suitable for use is stent-caged aneurysm treatment.”

U.S. Pat. No. 9,393,022 discloses “Embolic implants, delivery systems and methods of manufacture and delivery are disclosed. The subject implants are deployed in two stages. If sized properly as observed in the first stage, they are deployed to the second stage and detached. If not sized properly in/at the first stage, the implants are designed to be withdrawn and replaced with a more appropriately sized implant or another treatment option selected. Some of the implant configurations may be withdrawn even after the second stage deployment as well.”

Additional background art includes European Patent Document No. EP3136986, U.S. Patent Application No. 2019/0269414, U.S. Pat. No. 9,629,635, U.S. Patent Application No. 2017/0367708, U.S. patent application No. 20180140305, Japanese Patent Document No. JP2019526324, International Patent Application Publication No. WO2019/165360, U.S. patent application No. 20200038032, U.S. patent application No. 20190090884, International Patent Application Publication No. WO2017/106567, U.S. Patent Application No. 2019/0269411, U.S. Patent Application No. 2019/0343532, European Patent Document No. EP2157937, AU2009291548, and U.S. Pat. No. 10,335,153.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.
Example 1. An intravascular device comprising:
an expandable body having an expanded configuration and a contracted configuration, said expandable body in said expanded configuration having a convex curved shape having a base and walls; and
a connector;
wherein said connector is attached to said expandable body and recessed within said convex curved shape.
Example 2. The device according to Example 1, wherein said device comprises a support which provides a double layer to at least a portion of said expandable body.
Example 3. The device according to Example 2, wherein said support and said expandable body are connected by said connector.
Example 4. The device according to any one of Examples 1-2, wherein said expandable body is formed by a tubular structure which is closed at a proximal end by said connector.
Example 5. The device according to any one of Examples 1-4, comprising a radioactive source coupled to expandable body.
Example 6. The device according to any one of Examples 2-5, wherein one or both of said expandable body and said support include mesh.
Example 7. The device according to Example 6, wherein mesh fiber tips of ends of one or more portion of said device are blunted.
Example 8. The device according to any one of Examples 6-7, wherein at least some of mesh fibers are interconnected at a distal end of one or more portion of said device.
Example 9. The device according to any one of Examples 2-8, wherein said expandable body joins said connector from a distal end of said connector;
wherein said support joins said connector from a proximal end of said connector.
Example 10. The device according to any one of Examples 2-8, wherein said expandable body and said support are formed by a single tubular structure, folded to provide said expandable body and said support.
Example 11. The device according to any one of Examples 1-9, wherein said expandable body, in said expanded configuration is sufficiently resilient to resist collapse within an aneurysm.
Example 12. The device according to any one of Examples 1-10, wherein said expandable body includes a support portion reinforcing one or both of a portion of said walls and said base.
Example 13. The device according to Example 12, wherein said support includes at least a portion recessed within a volume formed by said walls.
Example 14. The device according to any one of Examples 12-13, wherein said support includes at least a portion extending around one or both of said walls and said base.
Example 15. The device according to any one of Examples 12-14, wherein said support extends from a distal end of said walls.
Example 16. The device according to any one of Examples 1-14, wherein at least one portion of said expandable body is sufficiently flexible to conform, at least partially, to an internal shape of an aneurysm.
Example 17. The device according to any one of Examples 1-16, wherein said expandable body is elastically expandable to said expanded configuration.
Example 18. An intravascular device comprising:
an expandable body including two layers and having an expanded configuration and a contracted configuration, where, when said expandable body is in said expanded configuration each of said two layers has a convex curved shape including walls extending from a base;
a connector connecting bases of said two layers.
Example 19. The intravascular device according to Example 18, wherein said connector is recessed within said convex curved shape, at least when said expandable body is in said expanded configuration.
Example 20. The intravascular device according to any one of Examples 18-19, wherein said connector defines a proximal end of said expandable body and wherein ends of said walls of one or both of said layers are not connected, providing an opening to a volume defined within walls of said first or said second layer.
Example 21. The intravascular device according to any one of Examples 18-20, wherein each said layer of said expandable body is formed by a tubular structure which is closed at a proximal end by said connector.
Example 22. The intravascular device according to any one of Examples 18-21, wherein said expandable body is formed by a single tubular structure, folded to form said bases, folding nesting a portion of the tubular structure within another portion of the tubular structure.
Example 23. The device according to any one of Examples 18-22, wherein said expandable body is constructed from mesh.
Example 24. The device according to Example 23, wherein mesh ends are blunted.
Example 25. The device according to any one of Examples 23-24, wherein at least a portion of mesh fibers are interconnected at an end region of said expandable body.
Example 26. The device according to any one of Examples 18-25, comprising a top portion at least partially enclosing a volume enclosed by said walls, where said base is disposed at a proximal end of said expandable body, said walls extend distally from said base and said top is disposed at a distal portion of said walls.
Example 27. An intravascular device comprising:
an expandable body having an expanded configuration and a contracted configuration, said expandable body comprising:
   a proximal end;
   a distal end; and
   walls connecting proximal and said distal end;
a connector disposed at said proximal end of said expandable body and connecting proximal ends of said walls;
a support disposed within a volume described by said walls and comprising:
   a support proximal end;
   a support distal end; and
   support walls extending from said connector at said support proximal end and extending towards said support distal end;
wherein said walls and said support walls are connected by said connector.
Example 28. The device of Example 27, wherein distal ends of said walls are not connected, providing an opening to said volume.
Example 29. The device of Example 28, wherein distal ends of said support walls are not connected, providing an opening to said volume.
Example 30. An intravascular device comprising:
an expandable body having an expanded configuration and a contracted configuration, said expandable body comprising:
   a first tubular portion;
   a second tubular portion nested within said first tubular portion;
   at least one connector closing a proximal opening of said first tubular portion and a proximal opening of said second tubular portion at a connection region; and
   wherein said first tubular portion enters said connection region from a first direction and said second tubular portion enters said connection region from a second direction.
Example 31. The device according to Example 30, wherein said first and said second tubular portions are formed by a single tubular portion folded at said connection region.
Example 32. An intravascular device comprising:
an expandable body having an expanded configuration and a contracted configuration, said expandable body comprising:
   a base; and
   walls extending from said base; and
a radioactive source connected to said base.
Example 33. The device of Example 32, wherein a dose emanating outside said expandable body, and provided by said radioactive source, when said expandable body is in in said expanded configuration, is less than 50 Gy.
Example 34. The device according to any one of Examples 32-33, where said radioactive source irradiates a dose of 10-40 Gy at 0-1 mm from an outer surface of the source.
Example 35. The device according to any one of Examples 32-34, wherein said radioactive source irradiates at an initial rate of 500-1000 mGy/hour.
Example 36. The device according to any one of Examples 32-35, wherein said source, when said expandable body is in said expanded configuration, is positioned in a central region of said expandable body.
Example 37. The device according to any one of Examples 32-35, wherein said source is attached to said expandable body by a holder, where said source is attached to said holder and said holder is attached to said body.
Example 38. The device according to Example 37, wherein said source is mounted onto said holder.
Example 39. The device according to any one of Examples 32-38, wherein said source is at least partially held within a lumen of said holder.
Example 40. The device according to any one of Examples 32-39, wherein a portion of said base connected to said source is recessed within said walls.
Example 41. The device according to any one of Examples 32-40, wherein said expandable body includes an opening sized and shaped to enable attachment of said source to said device.
Example 42. The device according to Example 41, wherein said opening is an opening in material mesh forming at least a portion of said expandable body.
Example 43. A method of treatment of aneurysm comprising:
positioning an expandable body within an aneurysm in position to cover an opening of the aneurysm;
irradiating said aneurysm using a source connected to said expandable body at radiation levels configured to stimulate formation of thrombi within the aneurysm and growth of tissue across the aneurysm opening.
Example 44. The method according to Example 43, wherein said positioning comprising delivering said expandable body intravascularly, in a contracted configuration.
Example 45. The method according to any one of Examples 43-44, wherein said positioning comprises expanding said expandable body within said aneurysm.
Example 46. The method according to any one of Examples 44-45, wherein said delivering comprises applying pressure to a holder connected to said expandable device.
Example 47. The method according to any one of Examples 43-46, comprising attaching a radioactive source to an expandable device, prior to positioning said device.
Example 48. The method according to Example 47, comprising collapsing said expandable device, prior to positioning said device and after attaching said radioactive source.
Example 49. The method according to any one of Examples 43-48, wherein said irradiating comprises irradiating a region of tissue at an opening of said aneurysm with a dose of 10-40 Gy.
Example 50. The method according to Example 49, wherein said irradiating comprises irradiating tissue outside said aneurysm with a dose of less than 50 Gy.
Example 101. An intravascular device comprising:
an expandable body having an expanded configuration and a contracted configuration, said expandable body comprising:
a base; and
walls extending from said base; and
a radioactive source connected to said base.
Example 102. The device of example 101, wherein said radioactive source provides a dose of 10-40 Gy to a portion of said expandable body, in said expanded configuration.
Example 103. The device of any one of Examples 101-102 wherein a dose emanating outside said expandable body, and provided by said radioactive source, when said expandable body is in in said expanded configuration, is less than 50 Gy.
Example 104. The device according to any one of Examples 101-103, where said radioactive source irradiates a dose of 10-40 Gy at 0-1 mm from an outer surface of the source.
Example 105. The device according to any one of Examples 101-104, where said radioactive source irradiates a dose of 0.1-50 Gy at 0-1 mm from an outer surface of the source.
Example 106. The device of Examples 101-105, where said radioactive source irradiates a dose of 20-150 Gy at 0-1 mm from an outer surface of the source.
Example 107. The device according to any one of Examples 104-106, wherein said radioactive source irradiates at an initial rate of 500-1000 mGy/hour.
Example 108. The device according to any one of Examples 104-106, wherein said radioactive source irradiates at an initial a rate of 25-500 mGy/hour.
Example 109. The device according to any one of Examples 104-106, wherein said radioactive source irradiates at an initial a rate of 0.1-100 mGy/hour.
Example 110. The device according to any one of Examples 101-109, wherein said expandable body, in said expanded configuration is sufficiently resilient to resist collapse within an aneurysm.
Example 111. The device according to any one of Examples 101-110, wherein said expandable body includes a support portion reinforcing one or both of a portion of said walls and said base.
Example 112. The device according to Example 111, wherein said support includes at least a portion recessed within a volume formed by said walls.
Example 113. The device according to any one of Examples 111-112, wherein said support includes at least a portion extending around one or both of said walls and said base.
Example 114. The device according to any one of Examples 111-113, wherein said support extends from a distal end of said walls.
Example 115. The device according to any one of Examples 111-113, wherein at least one portion of said expandable base is sufficiently flexible to conform, at least partially, to an internal shape of an aneurysm.
Example 116. The device according to any one of Examples 111-115, wherein said expandable body is elastically expandable to said expanded configuration.
Example 117. The device according to any one of Examples 111-116, wherein said source, when said expandable body is in said expanded configuration, is positioned in a central region of said expandable body.
Example 118. The device according to any one of Examples 111-117, wherein said source is attached to said expandable body by a holder, where said source is attached to said holder and said holder is attached to said body.
Example 119. The device according to Example 118, wherein said source is mounted onto said holder.
Example 120. The device according to any one of Examples 111-119, wherein said source is at least partially held within a lumen of said holder.
Example 121. The device according to any one of Examples 118-120, wherein said device includes a push-wire and said push-wire is connected to said holder.
Example 122. The device according to any one of examples 101-121, wherein a portion of said base connected to said source is recessed within said walls.
Example 123. The device according to any one of examples 101-122, wherein said walls are tubular.
Example 124. The device according to any one of examples 101-123, wherein said expandable body is constructed from mesh.
Example 125. The device according to any one of examples 101-124, wherein said expandable body includes an opening sized and shaped to enable attachment of said source to said device.
Example 126. The device according to Example 125, wherein said opening is an opening in material mesh forming at least a portion of said expandable body.
Example 127. The device according to any one of examples 101-126, comprising a top portion at least partially enclosing a volume enclosed by said walls, where said base is disposed at a proximal end of said expandable body, said walls extend distally from said base and said top is disposed at a distal portion of said walls.
Example 128. The device according to any one of examples 101-127, wherein at least a portion of said expandable body includes a double layered mesh.
Example 129. A method of treatment of aneurysm comprising:
positioning an expandable body within an aneurysm in position to cover an opening of the aneurysm;
irradiating said aneurysm using a source connected to said expandable body at radiation levels configured to stimulate formation of thrombi within the aneurysm and growth of tissue across the aneurysm opening.
Example 130. The method according to Example 129, wherein said positioning comprising delivering said expandable body intravascularly, in a contracted configuration.
Example 131. The method according to any one of examples 129-130, wherein said positioning comprises expanding said expandable body within said aneurysm.
Example 132. The method according to any one of examples 130-131, wherein said delivering comprises applying pressure to a holder connected to said expandable device.
Example 133. The method according to any one of examples 129-132, comprising attaching a radioactive source to an expandable device, prior to positioning said device.
Example 134. The method according to Example 133, comprising collapsing said expandable device, prior to positioning said device and after attaching said radioactive source.
Example 135. The method according to any one of examples 129-134, wherein said irradiating comprises irradiating a region of tissue at an opening of said aneurysm with a dose of 10-40 Gy.
Example 136. The method according to Example 135, wherein said irradiating comprises irradiating tissue outside said aneurysm with a dose of less than 50 Gy.
Example 137. An intravascular device comprising:
an expandable body having an expanded configuration and a contracted configuration, said expandable body in said expanded configuration having a convex curved shape; and
a connector;
wherein said connector is attached to said expandable body and recessed within said convex curved shape.
Example 138. The device according to Example 137, wherein said device comprises a support which provides a double layer to at least a portion of said expandable body.
Example 139. The device according to Example 138, wherein said support and said expandable body are connected by said connector.
Example 130. The device according to any one of examples 137-139, wherein said expandable body is formed by a tubular structure which is closed at a proximal end by said connector.
Example 131. The device according to Example 140, wherein said device comprises a support which provides a double layer to at least a portion of said expandable body;
wherein said support is formed by a tubular structure which is closed at a proximal end by said connector.
Example 132. The device according to any one of examples 138-141, wherein one or both of said expandable body and said support are include mesh.
Example 133. The device according to any one of examples 138-142, wherein said expandable body joins said connector from a distal end of said connector;
wherein said support joins said connector from a proximal end of said connector.
Example 134. The device according to any one of examples 141-143, wherein said expandable body and said support are formed by a single tubular structure, folded to provide said expandable body and said support.
Example 135. An intravascular device comprising:
a connector;
an expandable body having an expanded configuration and a contracted configuration, said expandable body comprising:
   a proximal end;
   a distal end; and
   walls connecting proximal and said distal end, said walls connected at said connector disposed at said proximal end;
a support disposed within a volume described by said walls and comprising:
   a support proximal end;
   a support distal end; and
walls
   support walls extending from said connector at said support proximal end towards said support distal end;
wherein said walls and said support walls are connected only by said connector.
Example 136. The device of Example 135, wherein distal ends of said walls are not connected, providing an opening to said volume.
Example 137. The device of Example 136, wherein distal ends of said support walls are not connected, providing an opening to said volume.
Example 138. An intravascular device comprising:
an expandable body having an expanded configuration and a contracted configuration, said expandable body comprising:
   a first tubular portion;
   a second tubular portion nested within said first tubular portion;
   at least one connector closing a proximal opening of said first tubular portion and a proximal opening of said second tubular portion at a connection region; and
   wherein said first tubular portion enters said connection region from a first direction and said second tubular portion enters said connection region from a second direction.
Example 139. The device according to Example 138, wherein said first and said second tubular portions are formed by a single tubular portion folded at said connection region.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified cross sectional schematic of an intravascular device system, according to some embodiments of the invention;

FIG. 2 is a flow chart of a method of treatment, according to some embodiments of the invention;

FIG. 3 is a flow chart of a method of treatment, according to some embodiments of the invention;

FIGS. 4A-H are simplified schematic cross sections showing treatment of an exemplary aneurysm, according to some embodiments of the invention;

FIGS. 5, 6, 7, 8, 9, 10 are simplified schematic cross sectional views of exemplary expandable devices;

FIG. 11 is a simplified view of an expandable device, according to some embodiments of the invention;

FIG. 12A is a simplified schematic view of an expandable device, from a distal direction, according to some embodiments of the invention;

FIG. 12B is a simplified schematic side view of an expandable device, according to some embodiments of the invention;

FIG. 12C is a simplified schematic cross sectional view of an expandable device, according to some embodiments of the invention;

FIG. 12D is a simplified schematic top view, from a proximal direction, of an expandable device, according to some embodiments of the invention;

FIG. 12E is a simplified schematic cross section of a collapsed expandable device, according to some embodiments of the invention;

FIG. 12F is a simplified schematic cross section of an expandable device within an aneurysm, according to some embodiments of the invention;

FIG. 12G is a simplified schematic cross sectional view of an expandable device, according to some embodiments of the invention;

FIG. 12H is a simplified schematic cross section of an expandable device within an aneurysm, according to some embodiments of the invention;

FIGS. 13A-B are simplified schematic sectional views of an expandable device 1302, according to some embodiments of the invention;

FIG. 13C is a simplified schematic cross sectional view of a portion of an expandable device, according to some embodiments of the invention;

FIG. 14A is a simplified schematic sectional view of an expandable device, according to some embodiments of the invention;

FIGS. 14B-C are simplified schematics of an expandable device, according to some embodiments of the invention;

FIG. 15A is a simplified schematic sectional view of an expandable device, according to some embodiments of the invention;

FIGS. 15B-C are simplified schematics of an expandable device, according to some embodiments of the invention;

FIG. 16A is a simplified schematic sectional view of an expandable device, according to some embodiments of the invention;

FIG. 16B is a simplified schematic view of an expandable device, according to some embodiments of the invention;

FIG. 17A is a simplified schematic of an expandable device, according to some embodiments of the invention;

FIG. 17B is a simplified schematic sectional view of an expandable device, according to some embodiments of the invention;

FIG. 18 is a simplified schematic cross sectional view of an expandable device, according to some embodiments of the invention;

FIG. 19A is a simplified schematic of a source holder, according to some embodiments of the invention;

FIG. 19B is a simplified schematic of a radioactive source, according to some embodiments of the invention;

FIG. 19C is a simplified schematic of a radioactive source connected to a source holder, according to some embodiments of the invention;

FIG. 20A is a simplified schematic of a source holder, according to some embodiments of the invention;

FIG. 20B is a simplified schematic of a radioactive source, according to some embodiments of the invention;

FIG. 20C is a simplified schematic of a radioactive source connected to a source holder, according to some embodiments of the invention;

FIG. 21 is a simplified schematic of a radioactive source, according to some embodiments of the invention;

FIG. 22A is a simplified schematic of a source holder, according to some embodiments of the invention;

FIG. 22B is a simplified schematic of a radioactive source, according to some embodiments of the invention;

FIG. 23 is a simplified schematic of a portion of an expandable device, according to some embodiments of the invention;

FIG. 24 is a simplified schematic of a portion of an expandable device, according to some embodiments of the invention;

FIG. 25 is a simplified schematic of a portion of an expandable device, according to some embodiments of the invention;

FIG. 26 is a simplified schematic of a portion of an expandable device, according to some embodiments of the invention;

FIG. 27 is a simplified schematic cross sectional view of an expandable device, according to some embodiments of the invention;

FIG. 28 is a simplified schematic cross sectional view of an expandable device, according to some embodiments of the invention;

FIG. 29A is a simplified schematic of an expandable device, according to some embodiments of the invention;

FIG. 29B is a simplified schematic cross section of an expandable device within an aneurysm, according to some embodiments of the invention;

FIG. 30A is a simplified schematic of an expandable device, according to some embodiments of the invention;

FIG. 30B is a simplified schematic cross section of an expandable device within an aneurysm, according to some embodiments of the invention;

FIG. 31 is a simplified schematic cross section of an expandable device within an aneurysm, according to some embodiments of the invention;

FIG. 32 is a simplified schematic cross section of an expandable device within an aneurysm, according to some embodiments of the invention;

FIG. 33 is a simplified schematic cross section of an expandable device within an aneurysm, according to some embodiments of the invention;

FIG. 34 is a simplified schematic cross section of an expandable device within an aneurysm, according to some embodiments of the invention;

FIG. 35 is a simplified schematic cross section of an expandable device, according to some embodiments of the invention;

FIG. 36 is a simplified schematic cross section of an expandable device, according to some embodiments of the invention;

FIG. 37 is a simplified schematic cross section of an expandable device, according to some embodiments of the invention;

FIG. 38 shows tables illustrating exemplary dose rates, according to some embodiments of the invention;

FIG. 39A is a flow chart of a method, according to some embodiments of the invention;

FIG. 39B is a simplified cross sectional schematic of an intravascular device system, according to some embodiments of the invention;

FIG. 39C is a simplified cross sectional schematic of an intravascular device system, according to some embodiments of the invention;

FIG. 40A is a simplified schematic cross sectional view of a device, according to some embodiments of the invention;

FIG. 40B is a simplified schematic cross sectional view of a portion of a device, according to some embodiments of the invention;

FIG. 40C is a simplified schematic cross sectional view of a portion of a device, according to some embodiments of the invention;

FIG. 41A is an image of a side view of a device, according to some embodiments of the invention;

FIG. 41B is an image of a device, according to some embodiments of the invention;

FIG. 42A is a simplified schematic cross sectional view of a device being delivered to a treatment region, according to some embodiments of the invention;

FIG. 42B is a simplified schematic cross sectional view of a device being deployed in a treatment region, according to some embodiments of the invention;

FIG. 42C is a simplified schematic cross sectional view of a deployed device in a treatment region, according to some embodiments of the invention;

FIG. 42D is an image of a deployed device in a treatment region, according to some embodiments of the invention;

FIG. 42E is a simplified schematic cross sectional view of a deployed device in a treatment region, according to some embodiments of the invention;

FIG. 42F is an image of a deployed device in a treatment region, according to some embodiments of the invention;

FIG. 43A is a simplified schematic cross sectional view of a device, according to some embodiments of the invention;

FIG. 43B is a simplified schematic cross sectional view of a portion of a device, according to some embodiments of the invention;

FIG. 43C is a simplified schematic cross sectional view of a portion of a device, according to some embodiments of the invention;

FIG. 44A is an image of a side view of a device, according to some embodiments of the invention;

FIG. 44B is an image of a device, according to some embodiments of the invention;

FIG. 45A is a simplified schematic cross sectional view of a device being delivered to a treatment region, according to some embodiments of the invention;

FIG. 45B is a simplified schematic cross sectional view of a device being deployed in a treatment region, according to some embodiments of the invention;

FIG. 45C is a simplified schematic cross sectional view of a deployed device in a treatment region, according to some embodiments of the invention;

FIG. 45D is a simplified schematic cross sectional view of a deployed device in a treatment region, according to some embodiments of the invention;

FIG. 45E is an image of a deployed device in a treatment region, according to some embodiments of the invention;

FIG. 46A is a simplified schematic cross sectional view of a device being delivered to a treatment region, according to some embodiments of the invention;

FIG. 46B is a simplified schematic cross sectional view of a device being deployed in a treatment region, according to some embodiments of the invention;

FIG. 46C is a simplified schematic cross sectional view of a deployed device in a treatment region, according to some embodiments of the invention;

FIG. 46D is an image of a deployed device in a treatment region, according to some embodiments of the invention;

FIG. 47A is a simplified schematic cross sectional view of a portion of a device, according to some embodiments of the invention;

FIG. 47B is a simplified schematic cross sectional view of a portion of a device, according to some embodiments of the invention;

FIG. 47C is a simplified schematic cross sectional view of a portion of a device, according to some embodiments of the invention;

FIG. 48A is a simplified schematic cross sectional view of a portion of a device, according to some embodiments of the invention;

FIG. 48B is a simplified schematic cross sectional view of a portion of a device, according to some embodiments of the invention;

FIG. 48C is a simplified schematic cross sectional view of a portion of a device, according to some embodiments of the invention;

FIG. 49A is a simplified schematic cross sectional view of a portion of a device, according to some embodiments of the invention;

FIG. 49B is a simplified schematic cross sectional view of a portion of a device, according to some embodiments of the invention;

FIG. 50A is a simplified schematic cross sectional view of a portion of a device, according to some embodiments of the invention;

FIG. 50B is a simplified schematic cross sectional view of a portion of a device, according to some embodiments of the invention;

FIG. 51 is a flowchart of a method, according to some embodiments of the invention;

FIG. 52 is an image of a portion of a device, according to some embodiments of the invention;

FIG. 53 is a simplified schematic cross sectional view of a device, according to some embodiments of the invention;

FIG. 54 is an image of a portion of a device, according to some embodiments of the invention; and

FIGS. 55A-D are simplified schematics device portions, according to some embodiments of the invention.

### DESCRIPTION OF SPECIFIC EMBODIMENTS OF THE INVENTION

The present invention, in some embodiments thereof, relates to treatment of intravascular abnormalities and, more particularly, but not exclusively, to treatment of aneurysm using an expandable intravascular device.

### Overview

A broad aspect of some embodiments of the invention relates to treatment of aneurysm using low level radiation to stimulate formation of thrombi within the aneurysm and/or neointima and/or vascular tissue growth at an opening of the aneurysm, to close the aneurysm. In some embodiments, thrombi formed within the aneurysm are prevented from exiting the aneurysm e.g. mechanically by obstruction of the aneurysm. Optionally, in some embodiments, additionally, blood flow into and/or out of the aneurysm is reduced, for example, by the mechanical obstruction. In some embodiments, closure of the aneurysm is achieved without blood flow (e.g. at peak arterial pressure) into and/or out of the aneurysm being reduced.

In some embodiments, an expandable intravascular device is positioned within the aneurysm and holds one or more radioactive source in a position to expose the aneurysm inner volume and/or aneurysm neck to suitable radiation levels whist holding the source at a distance away from healthy tissue that the radiation levels.

In some embodiments, the device includes an expandable body which has a range of expanded configurations associated with different aneurysm sizes. Where, in some embodiments, the expandable body conforms, at least partially to a size and/or shape of the aneurysm. In some embodiments, the device expandable body has a single relaxed expanded configuration e.g. when not under external forces of tissue and/or a delivery apparatus.

In some embodiments, the expandable device, when positioned inside an aneurysm, exposes tissue at an opening of the aneurysm (the “neck”) to a dose of 1-50 Gy, or 10-40 Gy, or lower or higher or intermediate doses or ranges (and/or doses as described in this section of the document). In some embodiments, the device does not expose tissue outside the aneurysm to more than 50 Gy, or more than 100 Gy, or more than 20 Gy, or lower or higher or intermediate ranges or doses. In some embodiments, the only regions of the device which are exposed to doses of more than 50 Gy (or more 100 Gy, or more than 20 Gy, or lower or higher or intermediate ranges or doses) are central (e.g. as defined elsewhere in this document) region/s of the device which, when the device is within an aneurysm relate only to an inner volume of the aneurysm and/or blood vessel and/or blood and/or thrombi.

In some embodiments, a highly radiative source is used, for example, where dosage at a source surface (or less than 1 mm, or less than 0.5 mm from a source surface) are above 50 Gy, or above 100 Gy, or above 300 Gy, or 300-500 Gy, or lower or higher or intermediate sources or ranges. In some embodiments, a highly radiative source provides, at 0-1 mm from an outer surface of the source, a dose of 100-500 Gy or lower or higher or intermediate ranges or doses. In some embodiments, a highly radiative source is positioned such that and/or shielded such that dosage at the neck of the device and/or at a proximal end of the device, and/or (and/or as described elsewhere in this document) and/or dosage outside the device is less than 50 Gy, or less than 100 Gy, or less than 20 Gy, or lower or higher or intermediate ranges or doses.

In some embodiments, dosage at the aneurysm neck is affected by one or more of:
Properties of the source, including size and/or shape and/or activity of the source.
A position of the source within the aneurysm, with respect to the neck e.g. distance of the source to the neck. Which, in some embodiments, is defined by a position of the source with respect to the expandable body, for the expanded configuration of the device for the particular aneurysm.
Shielding of the neck provided by the expandable body, where the shielding is affected by material properties of wires of the expandable body, and/or density of the mesh of the expandable device which, in some embodiments, is associated with expanded configuration of the device for the particular aneurysm.
Shielding of the neck provided by additional shielding elements of the device defined, for example, by the position and/or material characteristics of the element/s.

In some embodiments, dosage outside the aneurysm is also affected by these factors. For example one or more of:
Properties of the source, including size and/or shape and/or activity of the source.
A position of the source within the aneurysm, with respect to tissue portion/s outside the aneurysm e.g. distance therebetween. Which, in some embodiments, is defined by a position of the source with respect to the expandable body, for the expanded configuration of the device for the particular aneurysm.
Shielding of the tissue outside the aneurysm provided by the expandable body, where the shielding is affected by material properties of wires of the expandable body, and/or density of the mesh of the expandable device which, in some embodiments, is associated with expanded configuration of the device for the particular aneurysm.
Shielding of the tissue outside the aneurysm provided by additional shielding elements of the device defined, for example, by the position and/or material characteristics of the element/s.

In some embodiments, one or more of source property/ies, position within the device, shielding provided by the device, is selected to provide desired dosage at the aneurysm neck and outside the aneurysm. Where, in some embodiments, one or more of shielding and position of the source are selected for different aneurysm sizes. In some embodiments, one or more feature is fixed, and other feature/s are selected to provide the desired doses. For example, in some embodiments, an expandable device structure and/or material characteristics are selected and source and/or additional shielding element/s are then selected (and/or a position within the device thereof) to provide the desired dosages.

In some embodiments, one or more source is an element attached to the expandable device. Alternatively or additionally, in some embodiments, one or more portion of the expandable body (e.g. of the mesh) is itself radioactive. Where, in some embodiments, portion/s of the expandable device are treated (e.g. by one or more of the treatment methods as described elsewhere in this document) to be radioactive source/s e.g. as described within this document.

In some embodiments, material of the expandable device at least partially absorbs radiation emitted by source/s. In some embodiments, for example, at least a portion of the expandable device is positioned at and/or in close proximity (e.g. less than 0.1-1 mm, or less than 0.1-0.5 mm, or lower or higher or intermediate distances or ranges) away from walls of the aneurysm, the expandable body serving to, in some embodiments, protect tissue outside of the aneurysm from radiation emitted by the source. In some embodiments, an inner layer of a double layer device (e.g. including walls and support walls) acts to reduce radiation emitted by the source to a suitable levels for a region between the first layer closer to the source of the double layer and a second layer of the double layer. In some embodiments, a number of layers and/or a density of the expandable body is selected for different portions of the aneurysm, for example, in some embodiments, the source is less screened by the expandable body at a region of a neck of the aneurysm. In some embodiments, connecting region (e.g. line of sight) from the source to a neck region is less obstructed than region/s connecting the source to other portions of the device. Where, in some embodiments, less obstruction includes less layers of mesh (e.g. one layer as opposed to two layers) and/or porosity of the mesh and/or positioning of radiopaque element/s e.g. marker/s and/or connecting ring/s. In some embodiments, shielding provided by the expandable body itself enables use of higher strength sources and/or different type/s of radiation e.g. without (or minimally) damaging healthy tissue and/or tissue external to the aneurysm.

In some embodiments, shielding provided by the body of the device allows a single device to be used in a range of applications. For example, a device with a source disposed within a body of the expandable device. Where, when the expandable body is expanded within a small aneurysm has higher shielding properties (e.g. associated with denser packing of the mesh) protecting tissue outside the aneurysm and the same device, in some embodiments, is used for a larger aneurysm, the source being disposed further away from tissue outside of the aneurysm balancing reduced screening by the expanded less dense mesh.

In some embodiments, the expandable intravascular device covers, at least partially, and, in some embodiments, fully, an opening of the aneurysm. For example, with a mesh where, in some embodiments, a mesh pore size is selected to prevent outflow of thrombi over a certain size from the aneurysm.

In an exemplary embodiment, the aneurysm is a wide neck cerebral saccular aneurysm. In some embodiments, the aneurysm has dimensions of 3-10 mm average and/or maximum dimension with an aneurysm neck maximum and/or average cross sectional dimension of 3-8 mm, or at least 1-mm, or at least 2 mm, or at least 3 mm, or at least 4 mm, or lower or higher or intermediate sizes or ranges. In some embodiments, a dome to neck ratio of the aneurysm is less than 2, or is 0.1-3, or is lower or higher or intermediate ranges or ratios.

In some embodiments, the aneurysm is a large or giant aneurysm with a maximal and/or average dimension of 10-25 mm or lower or higher or intermediate dimensions or ranges, with, for example, dome to neck ratio of 0.1-3, or lower or higher or intermediate ranges or ratios.

In some embodiments, the aneurysm sac and/or aneurysm neck are exposed to low doses and/or low dose rates of ionizing irradiation, for example, as described later in this document.

An aspect of some embodiments of the invention relates to an intravascular device including an expandable body having walls and a base, with at least one radioactive source connected to the body.

In some embodiments, the expandable body is provided separately to the radioactive source/s, for example, the radioactive source being affixed to the expandable body prior to positioning of the device intravascularly. Potential benefits being decoupling of shelf life of the source and expandable body and/or the need to store only the sources in shielded containers.

In some embodiments, the expandable intravascular device is delivered to an aneurysm and expanded within a volume of the aneurysm, positioning the radioactive source within the aneurysm.

In some embodiments, once the device is expanded within the aneurysm, the base covers an opening of the aneurysm. For example, in some embodiments, the base and/or walls of the device, in an expanded configuration, are sized and/or shaped to cover the aneurysm.

In some embodiments, a device expanded configuration is over-sized, in one or more dimension, with respect to the aneurysm. A potential benefit being that force of the device on the aneurysm and/or resulting friction and/or close fitting of the device to the aneurysm prevents movement of the device, once expanded. A potential benefit of force and/or friction between the device and aneurysm is wounding and/or irritation to the aneurysm which, in some embodiments, increase a rate of healing and/or treatment. In some embodiments, over-sizing is in one or more direction perpendicular to a plane of the aneurysm opening, and/or in an aneurysm opening to dome direction, which direction designations are herein termed, with respect to the device, a proximal-distal direction and/or a length direction of the expandable device. Additionally, or alternatively, in some embodiments, over-sizing is in one or more direction parallel to a plane of the aneurysm volume and/or perpendicular to the proximal-distal and/or parallel to a width and/or depth direction of the device.

In some embodiments, once the device is expanded within the aneurysm, a distal portion of the device adjacent to and/or in contact with aneurysm walls acts to hold the device in position covering the opening of the aneurysm. For example, where contact of the distal portion of the device (e.g. with aneurysm dome), in some embodiments, is arranged by selection of the device to be over-sized with respect to dimension/s of the aneurysm e.g. in an aneurysm neck to dome direction. In some embodiments, the device is configured, for example, a length of the device extending distally from the base is configured to dispose a distal portion of the device at walls of the aneurysm. Where, in some embodiments, the device is elastically expandable in a proximal-distal direction and a relaxed length of the device is larger than a length of the aneurysm e.g. distance from the aneurysm opening to facing inner walls of the aneurysm.

In some embodiments, the expandable body, when at least partially expanded and positioned within an aneurysm, is sufficiently resistant to forces of tissue on the expandable body to prevent collapse of the expandable body. In some embodiments, the base and/or walls are configured to resist collapse of the expandable device e.g. under tissue forces of the aneurysm and/or surrounding tissue. In some embodiments, thrombi formed within the aneurysm prevent collapse of the aneurysm and/or of the expandable body. A potential benefit being reduced likelihood of dislodgement of the device from the aneurysm and/or the aneurysm collapsing leaving a remnant aneurysm neck. In some embodiments, the base and/or walls of the expandable body resist collapse of the aneurysm onto the source. A potential benefit being that healthy tissue surrounding the aneurysm is not irradiated at the radiation levels selected for treatment of the aneurysm.

In some embodiments, one or more portion of the expandable body is sufficiently flexible that the expandable body conforms, at least partially, to a shape of the aneurysm. For example, in some embodiments, portion/s of the expandable body walls (e.g. distal portion/s) of the walls conform, at least partially, to a shape of the aneurysm. A potential benefit being reduced likelihood of the device dislodging from position within the aneurysms.

In some embodiments, the expandable body of the device is self-expanding. For example, elastically self-expanding upon exiting (e.g. being pushed out of) a delivery catheter. In some embodiments, the elastic expanding force of the expandable body is sufficient to resist collapse of the expandable body, potentially reducing the chance of dislodgement of the device from the aneurysm lumen.

In some embodiments, the expandable body is not self-expanding or is only partially self-expanding. In some embodiments, for example, the expandable is balloon expanded e.g. by inflating a balloon positioned within the expandable body.

In some embodiments, the source is connected to the expandable body such that the source when the expandable body is in a relaxed expanded configuration (not under externally applied force/s) is positioned at a central region of the expandable body, in one or more direction. In some embodiments, the source is connected to the expandable body such that the source, once the expandable body is positioned within the aneurysm, is positioned in a central region of the aneurysm. For example, in some embodiments, the source is connected to the expandable body such that the source when the expandable body is deployed within an aneurysm (optionally at least partially conforming to the aneurysm shape) is positioned at a central region of the expandable body, in one or more direction.

In some embodiments, a center of the source is positioned within a central 5-80%, or 5-50%, or 5-20%, or lower or higher or intermediate percentages or ranges of the device volume. In some embodiments, the source is positioned within a central region of the expandable structure in one or more direction. For example, in some embodiments, the source (e.g. a center of a cross section of the source taken parallel to the base) is positioned centrally with respect to a base of the expandable device e.g. within a central 5-80%, or 5-50%, or 5-20%, or lower or higher or intermediate percentages or ranges of the base. For example, in some embodiments, the source (e.g. a center of a cross section of the source taken parallel to the walls) is positioned centrally with respect to walls of the expandable device e.g. within a central 5-80%, or 5-50%, or 5-20%, or lower or higher or intermediate percentages or ranges of the walls.

In an exemplary embodiment, the source is connected to a holder which is attached to the expandable body. In some embodiments, the source is connected to the holder, shortly prior to use of the device in treatment. A potential benefit being de-coupling of the source expiry-date and/or lifetime from that of the expandable body.

In some embodiments, the holder also provides mechanical support to the expandable body. Where, in some embodiments, the holder is used in delivery of the device e.g. intravascularly.

In some embodiments, one or more connection between the expandable body and other element/s is located within the expandable body. For example, within a central 80-99%, or 80-95% of the expandable body. Where connections include, for example, connection between the expandable body and a holder and/or source and/or connection between the expandable body and a portion of a delivery apparatus e.g. push wire.

In some embodiments, a portion of the expandable body is at least partially recessed within a volume of the device defined by walls of the device. For example, at least a portion of the base and/or at least a portion of a top of the expandable device is recessed within a lumen defined by the walls. In some embodiments, recessing is of a portion of the device including one or more connector. For example, a connector holding a portion of the device closed e.g. base closing connector and/or top closing connector. For example, a connector to a push-wire and/or other delivery apparatus portion/s. For example, a connector to a source and/or source holder.

In some embodiments, recessing of a portion to which the source is connected (e.g. base and/or top) moves connection of the source closer to a center of the device walls. A potential advantage of recessing connection of the source to the body is reduced disruption of the source connection to blood flow within the main vessel to which the aneurysm is a deformity.

In some embodiments, a connection of the expandable device to a delivery apparatus (e.g. a push-wire) is recessed within the device.

In an exemplary embodiment, connection of the source and of the delivery apparatus is at the same portion and/or region of the base, which, in some embodiments, is recessed within the expandable body.

In some embodiments, the expandable body includes a top. Where in some embodiments, the top extends from the walls and at least partially encloses a lumen formed by the walls. In some embodiments, the top is closed. In some embodiments, the top includes an opening, which, in some embodiments, provides access for attachment of the source to the expandable body e.g. attachment to the base and/or to the base via a holder attached to the base.

In some embodiments, one or more portion of the expandable structure is multi-layer. Where, for example, in some embodiments, a portion of the base and/or of a top is recessed within a tubular structure defined by the walls. In some embodiments, the base and/or walls are surrounded (e.g. proximally) by a support structure. A potential advantage of a multi-layer structure include increased resilience to collapse of the structure and/or increased scaffolding providing surfaces for growth of thrombi.

In some embodiments, the top is at least partially recessed within the volume described by the walls. A potential benefit of a recessed base and/or recessed top is increased resistance of the expandable device to compression, for example, of the region of the expandable body where the recessed portion/s reinforce (e.g. form a double layer with) walls of the device. In some embodiments, the increased resistance is to compression in a direction perpendicular to a central longitudinal axis of the walls (e.g. tubular walls), for example a radial direction for a cylindrical type expandable body.

A potential benefit of recessed portion/s is increased rate of forming of thrombi within the aneurysm associated with increased scaffolding provided by the recessed portion/s.

In some embodiments, recessed portion/s are formed by folding of material extending from walls of the expandable device within the lumen defined by the walls. For example, a tubular structure forms the walls and end portion/s of the walls are then folded into the tubular structure. In some embodiments, folded end/s are not recessed but extend externally around the walls. For example providing mechanical strengthening of the walls and/or a double layer of mesh providing scaffolding and/or restriction of blood flow e.g. at the aneurysm opening. In some embodiments, folded and/or double layer portion/s are not used at a base of the device, preventing additional material from screening opening of the aneurysm from emitted radiation. Alternatively, in some embodiments, a base includes more than one layer of mesh which in some embodiments, is formed by folding of the material e.g. of the base and/or of walls the folded material extending to form a double layered portion. Potential benefits being increased restriction of blood flow and/or increased mechanical resilience.

In some embodiments, one or more portion of the expandable body is formed from a mesh e.g. a wire mesh. In some embodiments, the mesh material is biocompatible and/or coated in biocompatible material. In an exemplary embodiment, the wire mesh includes nitinol. In some embodiments, pore size of the mesh is selected to reduce blood flow into the aneurysm and/or prevent thrombi from escaping from within the aneurysm. Further exemplary details of the mesh structure are described in the section below entitled “Exemplary expandable body materials”.

In some embodiments, one or more portion of the expandable body includes more than one layer of mesh e.g. is double-layered. Potential benefits of additional layers include increased mechanical resilience (e.g. associated with resistance of the device to collapse) and/or reduced pore size and/or increased scaffolding providing a surface for thrombi to grow within the aneurysm and/or for enothelization at the aneurysm neck. In some embodiments, the base portion is formed at least partially by a multi-layer mesh (e.g. double layer).

In some embodiments, the source is attached to the expandable body by a holder. In some embodiments, the holder is formed of ionizing radiation resistant material for example, an ionizing radiation resistant polymer, e.g. PEEK and/or polyimide. In some embodiments, the source is placed within a lumen of the holder. Where, in some embodiments, once the source is within the lumen of the holder the lumen is closed and/or constricted to prevent movement of the source.

In some embodiments, the source is attached externally to the holder. In an exemplary embodiment, the source is stretched onto the holder, reactive force of the stretching holding the source to the holder.

In some embodiments, the holder extends from the base distally and the source is attached to the holder at a distal portion of the holder.

In some embodiments, the holder provides mechanical support to the intravascular device. For example, in some embodiments, delivery of the device is by applying pressure to the holder, for example, through a push-wire connected to the holder. In some embodiments, the holder is elongate. In some embodiments, the holder is flexible axially but resists deformation in a direction of elongation of the holder. Potentially axial flexibility and/or resistance to deformation in a direction of elongation enable delivery of the device e.g. the collapsed device through a delivery lumen (e.g. catheter).

In some embodiments, the holder is directly connected to the expandable body. For example, by welding. For example, by connecting (e.g. adhering, screwing together) two pieces of the holder with the body disposed therebetween.

Alternatively or additionally, in some embodiments, the expandable body is attached to the holder by a portion of the expandable body being disposed between the holder and a connector. In an exemplary embodiment, the connector is ring-shaped and is optionally radiopaque. In some embodiments, the connector is attached to the expandable body by one or more of crimping, gluing, welding, and quenching. In some embodiments, the connector is attached to the expandable body and holder together e.g. by one or more of crimping, gluing, welding, and quenching. In an exemplary embodiment, the connector is quenched onto mesh of the expandable body and the holder together.

In some embodiments, the expandable body is attached to the holder at one or more place. For example, in some embodiments, both the expandable body base and the expandable body top are attached to the holder. For example, each of the base and the ring by a different connector.

In some embodiments, the walls and base are reinforced by a support (which in some embodiments, includes additional walls optionally connected to a base). In some embodiments, the support is proximal to the expandable body walls and base and optionally connected to the delivery apparatus. Optionally, in some embodiments, the support is attached to the holder.

In some embodiments, the expandable device walls form a tubular shape. In some embodiments, a cross section of the tubular shape is constant along a length of the walls (e.g. varies by at most 10%, or 5% or lower or higher or intermediate percentages). In some embodiments, the cross section is symmetrical about one or more axis e.g. in some embodiments, circular.

In some embodiments, a cross section of the tubular shape changes along the length of the walls e.g. increases in a distal direction, for example, where the expanded device includes sloped walls e.g. has a truncated cone shape extending from the base.

In some embodiments, a cross section of the tubular shape of the walls increases rapidly along a length of the walls, for example, to form a flattened cup shape with the base. For example, in some embodiments, the wall cross section at least doubles, or increases by 1.5-50 times, or 5-20, or about 10 times along the wall length.

In some embodiments, the expandable device is delivered pressure applied to a push-wire, after delivery, the push-wire is detached and removed. In some embodiments, detachment of the push-wire is by electrolytic weakening of a connection between the push-wire and the expandable device. Where, in some embodiments, the push-wire is electrically conducting and a current applied to the push-wire acts to weaken the connection enabling the push-wire to be removed from the treatment site, while leaving the expandable device in situ. Alternatively or additionally, in some embodiments, disconnection of the push-wire is via electrothermal weakening of the connection between the push-wire and the expandable device e.g. where an electrical current is used to heat the connection to weaken it. Alternatively or additionally, in some embodiments, detachment of the push-wire from the device includes a mechanical release e.g. where tension is changed on an elongate element (in some embodiments, the push-wire) to detach the push-wire from the expandable device. In some embodiments, attachment of the push-wire is to the holder. Where, in some embodiments, the holder is electrically insulating, potentially preventing electrical stimulation (e.g. for electrolytic and/or electrothermic detachment of the push-wire from affecting the expandable device and/or adjacent and/or enclosed tissue).

In some embodiments, the device is configured to be reattached to an apparatus for movement of the device. For example, to be reattached to a push-wire. In some embodiments, the device is extracted and/or re-positioned after reattachment e.g. of a push-wire. In some embodiments, a push-wire is magnetically attached and/or detached from the expandable body. For example, via the holder.

In some embodiments, the radioactive source is removed from within the aneurysm e.g. after treatment has been completed, for example, leaving portion/s of the expandable body of the device in situ. For example, in some embodiments, the holder is re-attached to a push-wire and the holder and source are removed from the device.

In some embodiments, the expandable device includes one or more radiopaque marker. For example, in some embodiments, one or more connector (e.g. connection ring) includes radiopaque material. For example, in some embodiments, the mesh of the device itself includes radiopaque material.

In some embodiments, the source is positioned, with respect to radiopaque marker/s such that the marker/s do not shield radiation from being emitted in particular direction/s and/or to particular region/s of tissue. For example, in some embodiments, a marker (e.g. a ring attachment) is positioned distally of the source, potentially preventing shielding of the aneurysm neck region from radiation, by the marker.

In some embodiments, a radioactive source is directionally shielded in one or more direction. For example, to prevent radiating sensitive tissue outside the aneurysm e.g. the optic nerve. In some embodiments, shielding portion/s form radiopaque markers e.g. potentially enabling a user to verify correct positioning of the device and/or shield.

In some embodiments, pore size of one or more portion of the device is selected to allow insertion of additional element/s. For example, for insertion of aneurysm treatment coil/s of the art.

Although devices and methods have been described with respect to treatment of aneurysm, in some embodiments, devices and/or methods as described in this document are used for treatment of other tissue. For example for closing lumen/s of other body portions, e.g. left atrial appendage closure.

In some embodiments, devices as described in this document do not include radioactive source/s. For example, in some embodiments, structures as described are used for treatment without a radioactive source. In some embodiments, a device including a source is deployed, and the source is later removed e.g. leaving the device in situ. In some embodiments, a device without a source is deployed.

Optionally, in some embodiments, after a treatment time period, one or more radioactive source is coupled to the device e.g. a device initially lacking source/s. Where, in some embodiments, removal and/or addition of radioactive source/s is based on clinical assessment of efficacy of treatment and/or pre-determined in a treatment plan.

A broad aspect of some embodiments of the invention relates to treatment of an aneurysm by deploying an expandable device into the aneurysm. In some embodiments, the device has a double layer (e.g. a body layer and a support layer) which, when the device is deployed, covers an opening of the aneurysm. In some embodiments, one or both layers are formed of mesh e.g. wire mesh.

In some embodiments, the base and walls form a curved surface which, when the device is deployed within an aneurysm is presented to blood flow of vessel/s hosting the aneurysm. In some embodiments, the second layer of the double layer is provided by a support.

In some embodiments, distal portions of the support and the body are not connected and/or the support and body are only connected at the connection region. A potential benefit being ease of delivery through vessels (e.g. tortuous blood vessels) as, for example, in some embodiments, transitioning through turns support and body are able to move with respect to each other.

In some embodiments, the device includes an expandable body which has a range of expanded configurations associated with different aneurysm sizes. Where, in some embodiments, the expandable body conforms, at least partially to a size and/or shape of the aneurysm. In some embodiments, the device expandable body has a single relaxed expanded configuration e.g. when not under external forces of tissue and/or a delivery apparatus.

In some embodiments, the expandable intravascular device covers, at least partially, and, in some embodiments, fully, an opening of the aneurysm. For example, with a mesh where, in some embodiments, a mesh pore size is selected to prevent outflow of thrombi over a certain size from the aneurysm.

In an exemplary embodiment, the aneurysm is a wide neck cerebral saccular aneurysm. In some embodiments, the aneurysm has dimensions of 3-10 mm average and/or maximum dimension with an aneurysm neck maximum and/or average cross sectional dimension of 3-8 mm, or at least 1-mm, or at least 2 mm, or at least 3 mm, or at least 4 mm, or lower or higher or intermediate sizes or ranges. In some embodiments, a dome to neck ratio of the aneurysm is less than 2, or is 0.1-3, or is lower or higher or intermediate ranges or ratios.

In some embodiments, the aneurysm is a large or giant aneurysm with a maximal and/or average dimension of 10-25 mm or lower or higher or intermediate dimensions or ranges, with, for example, dome to neck ratio of 0.1-3, or lower or higher or intermediate ranges or ratios.

In some embodiments, once the device is expanded within the aneurysm, the base covers an opening of the aneurysm. For example, in some embodiments, the base and/or walls of the device, in an expanded configuration, are sized and/or shaped to cover the aneurysm.

In some embodiments, a device expanded configuration is over-sized, in one or more dimension, with respect to the aneurysm. A potential benefit being that force of the device on the aneurysm and/or resulting friction and/or close fitting of the device to the aneurysm prevents movement of the device, once expanded. A potential benefit of force and/or friction between the device and aneurysm is wounding and/or irritation to the aneurysm which, in some embodiments, increase a rate of healing and/or treatment. In some embodiments, over-sizing is in one or more direction perpendicular to a plane of the aneurysm opening, and/or in an aneurysm opening to dome direction, which direction designations are herein termed, with respect to the device, a proximal-distal direction and/or a length direction of the expandable device. Additionally, or alternatively, in some embodiments, over-sizing is in one or more direction parallel to a plane of the aneurysm volume and/or perpendicular to the proximal-distal and/or parallel to a width and/or depth direction of the device.

In some embodiments, once the device is expanded within the aneurysm, a distal portion of the device adjacent to and/or in contact with aneurysm walls acts to hold the device in position covering the opening of the aneurysm. For example, where contact of the distal portion of the device (e.g. with aneurysm dome), in some embodiments, is arranged by selection of the device to be over-sized with respect to dimension/s of the aneurysm e.g. in an aneurysm neck to dome direction. In some embodiments, the device is configured, for example, a length of the device extending distally from the base is configured to dispose a distal portion of the device at walls of the aneurysm. Where, in some embodiments, the device is elastically expandable in a proximal-distal direction and a relaxed length of the device is larger than a length of the aneurysm e.g. distance from the aneurysm opening to facing inner walls of the aneurysm.

In some embodiments, the expandable body, when at least partially expanded and positioned within an aneurysm, is sufficiently resistant to forces of tissue on the expandable body to prevent collapse of the expandable body. In some embodiments, the base and/or walls are configured to resist collapse of the expandable device e.g. under tissue forces of the aneurysm and/or surrounding tissue. In some embodiments, thrombi formed within the aneurysm prevent collapse of the aneurysm and/or of the expandable body. A potential benefit being reduced likelihood of dislodgement of the device from the aneurysm and/or the aneurysm collapsing leaving a remnant aneurysm neck. In some embodiments, the base and/or walls of the expandable body resist collapse of the aneurysm onto the source. A potential benefit being that healthy tissue surrounding the aneurysm is not irradiated at the radiation levels selected for treatment of the aneurysm.

In some embodiments, one or more portion of the expandable body is sufficiently flexible that the expandable body conforms, at least partially, to a shape of the aneurysm. For example, in some embodiments, portion/s of the expandable body walls (e.g. distal portion/s) of the walls conform, at least partially, to a shape of the aneurysm. A potential benefit being reduced likelihood of the device dislodging from position within the aneurysms.

In some embodiments, the expandable body of the device is self-expanding. For example, elastically self-expanding upon exiting (e.g. being pushed out of) a delivery catheter. In some embodiments, the elastic expanding force of the expandable body is sufficient to resist collapse of the expandable body, potentially reducing the chance of dislodgement of the device from the aneurysm lumen.

In some embodiments, the expandable body is not self-expanding or is only partially self-expanding. In some embodiments, for example, the expandable is balloon expanded e.g. by inflating a balloon positioned within the expandable body.

In some embodiments, one or more connection between the expandable body and other element/s is located within the expandable body. For example, within a central 80-99%, or 80-95% of the expandable body. Where connections include, for example, connection between the expandable body and a holder and/or source and/or connection between the expandable body and a portion of a delivery apparatus e.g. push wire.

In some embodiments, a portion of the expandable body is at least partially recessed within a volume of the device defined by walls of the device. For example, at least a portion of the base and/or at least a portion of a top of the expandable device is recessed within a lumen defined by the walls. In some embodiments, recessing is of a portion of the device including one or more connector. For example, a connector holding a portion of the device closed e.g. base closing connector and/or top closing connector. For example, a connector to a push-wire and/or other delivery apparatus portion/s. For example, a connector to a source and/or source holder.

In some embodiments, recessing of a portion to which the source is connected (e.g. base and/or top) moves connection of the source closer to a center of the device walls. A potential advantage of recessing connection of the source to the body is reduced disruption of the source connection to blood flow within the main vessel to which the aneurysm is a deformity.

In some embodiments, a connection of the expandable device to a delivery apparatus (e.g. a push-wire) is recessed within the device.

In some embodiments, the expandable body includes a top. Where in some embodiments, the top extends from the walls and at least partially encloses a lumen formed by the walls. In some embodiments, the top is closed. In some embodiments, the top includes an opening, which, in some embodiments, provides access for attachment of the source to the expandable body e.g. attachment to the base and/or to the base via a holder attached to the base.

In some embodiments, one or more portion of the expandable structure is multi-layer. Where, for example, in some embodiments, a portion of the base and/or of a top is recessed within a tubular structure defined by the walls. In some embodiments, the base and/or walls are surrounded (e.g. proximally) by a support structure. A potential advantage of a multi-layer structure include increased resilience to collapse of the structure and/or increased scaffolding providing surfaces for growth of thrombi.

In some embodiments, the top is at least partially recessed within the volume described by the walls. A potential benefit of a recessed base and/or recessed top is increased resistance of the expandable device to compression, for example, of the region of the expandable body where the recessed portion/s reinforce (e.g. form a double layer with) walls of the device. In some embodiments, the increased resistance is to compression in a direction perpendicular to a central longitudinal axis of the walls (e.g. tubular walls), for example a radial direction for a cylindrical type expandable body.

A potential benefit of recessed portion/s is increased rate of forming of thrombi within the aneurysm associated with increased scaffolding provided by the recessed portion/s.

In some embodiments, recessed portion/s are formed by folding of material extending from walls of the expandable device within the lumen defined by the walls. For example, a tubular structure forms the walls and end portion/s of the walls are then folded into the tubular structure. In some embodiments, folded end/s are not recessed but extend externally around the walls. For example providing mechanical strengthening of the walls and/or a double layer of mesh providing scaffolding and/or restriction of blood flow e.g. at the aneurysm opening. In some embodiments, folded and/or double layer portion/s are not used at a base of the device, preventing additional material from screening opening of the aneurysm from emitted radiation. Alternatively, in some embodiments, a base includes more than one layer of mesh which in some embodiments, is formed by folding of the material e.g. of the base and/or of walls the folded material extending to form a double layered portion. Potential benefits being increased restriction of blood flow and/or increased mechanical resilience.

In some embodiments, one or more portion of the expandable body is formed from a mesh e.g. a wire mesh. In some embodiments, the mesh material is biocompatible and/or coated in biocompatible material. In an exemplary embodiment, the wire mesh includes nitinol. In some embodiments, pore size of the mesh is selected to reduce blood flow into the aneurysm and/or prevent thrombi from escaping from within the aneurysm. Further exemplary details of the mesh structure are described in the section below entitled “Exemplary expandable body materials”.

In some embodiments, one or more portion of the expandable body includes more than one layer of mesh e.g. is double-layered. Potential benefits of additional layers include increased mechanical resilience (e.g. associated with resistance of the device to collapse) and/or reduced pore size and/or increased scaffolding providing a surface for thrombi to grow within the aneurysm and/or for endothelization at the aneurysm neck. In some embodiments, the base portion is formed at least partially by a multi-layer mesh (e.g. double layer).

In some embodiments, the holder provides mechanical support to the intravascular device. For example, in some embodiments, delivery of the device is by applying pressure to the holder, for example, through a push-wire connected to the holder. In some embodiments, the holder is elongate. In some embodiments, the holder is flexible axially but resists deformation in a direction of elongation of the holder. Potentially axial flexibility and/or resistance to deformation in a direction of elongation enable delivery of the device e.g. the collapsed device through a delivery lumen (e.g. catheter).

In some embodiments, the holder is directly connected to the expandable body. For example, by welding. For example, by connecting (e.g. adhering, screwing together) two pieces of the holder with the body disposed therebetween.

Alternatively or additionally, in some embodiments, the expandable body is attached to the holder by a portion of the expandable body being disposed between the holder and a connector. In an exemplary embodiment, the connector is ring-shaped and is optionally radiopaque. In some embodiments, the connector is attached to the expandable body by one or more of crimping, gluing, welding, and quenching. In some embodiments, the connector is attached to the expandable body and holder together e.g. by one or more of crimping, gluing, welding, and quenching. In an exemplary embodiment, the connector is quenched onto mesh of the expandable body and the holder together.

In some embodiments, the expandable body is attached to the holder at one or more place. For example, in some embodiments, both the expandable body base and the expandable body top are attached to the holder. For example, each of the base and the ring by a different connector.

In some embodiments, the walls and base are reinforced by a support (which in some embodiments, includes additional walls optionally connected to a base). In some embodiments, the support is proximal to the expandable body walls and base and optionally connected to the delivery apparatus. Optionally, in some embodiments, the support is attached to the holder.

In some embodiments, the expandable device walls form a tubular shape. In some embodiments, a cross section of the tubular shape is constant along a length of the walls (e.g. varies by at most 10%, or 5% or lower or higher or intermediate percentages). In some embodiments, the cross section is symmetrical about one or more axis e.g. in some embodiments, circular.

In some embodiments, a cross section of the tubular shape changes along the length of the walls e.g. increases in a distal direction, for example, where the expanded device includes sloped walls e.g. has a truncated cone shape extending from the base.

In some embodiments, a cross section of the tubular shape of the walls increases rapidly along a length of the walls, for example, to form a flattened cup shape with the base. For example, in some embodiments, the wall cross section at least doubles, or increases by 1.5-50 times, or 5-20, or about 10 times along the wall length.

In some embodiments, the expandable device is delivered pressure applied to a push-wire, after delivery, the push-wire is detached and removed. In some embodiments, detachment of the push-wire is by electrolytic weakening of a connection between the push-wire and the expandable device. Where, in some embodiments, the push-wire is electrically conducting and a current applied to the push-wire acts to weaken the connection enabling the push-wire to be removed from the treatment site, while leaving the expandable device in situ. Alternatively or additionally, in some embodiments, disconnection of the push-wire is via electrothermal weakening of the connection between the push-wire and the expandable device e.g. where an electrical current is used to heat the connection to weaken it. Alternatively or additionally, in some embodiments, detachment of the push-wire from the device includes a mechanical release e.g. where tension is changed on an elongate element (in some embodiments, the push-wire) to detach the push-wire from the expandable device. In some embodiments, attachment of the push-wire is to the holder. Where, in some embodiments, the holder is electrically insulating, potentially preventing electrical stimulation (e.g. for electrolytic and/or electrothermic detachment of the push-wire from affecting the expandable device and/or adjacent and/or enclosed tissue).

In some embodiments, the device is configured to be reattached to an apparatus for movement of the device. For example, to be reattached to a push-wire. In some embodiments, the device is extracted and/or re-positioned after reattachment e.g. of a push-wire. In some embodiments, a push-wire is magnetically attached and/or detached from the expandable body.

For example, via the holder.

In some embodiments, the expandable device includes one or more radiopaque marker. For example, in some embodiments, one or more connector (e.g. connection ring) includes radiopaque material. For example, in some embodiments, the mesh of the device itself includes radiopaque material.

In some embodiments, pore size of one or more portion of the device is selected to allow insertion of additional element/s. For example, for insertion of aneurysm treatment coil/s of the art.

An aspect of some embodiments of the invention relates to a low delivery profile (also herein termed “crimped profile”) double layered aneurysm treatment device.

Where, when crimped (e.g. within a delivery catheter), the device maximal cross sectional extent (e.g. diameter) is 200-1400 μm, or 300-1000 μm, or 300-700 μm, or lower or higher or intermediate distances or ranges. In some embodiments, a minimal delivery profile of the device is defined by a cross sectional extent of a connection region of the device and, optionally a layer thickness/es of one or both of the double layers. Where, in some embodiments, the connection region maximal cross section dimension is 100-700 μm, or 200-500 μm, or lower or lower or higher or intermediate distances or ranges. In some embodiments, layer thickness of one or more layer of the device is 50-200 μm, or 50-150 μm, or 50-100 μm, or lower or higher or intermediate distances or ranges. In some embodiments, a double layered device, at a connection region of the device, when the device is in a crimped configuration, has at most, a single surrounding layer of device mesh material (e.g. body and/or support layer material).

In some embodiments, support portion/s of the device extend from the connection region in a different direction to that of body portion/s of the device. In some embodiments, when in a crimped configuration, the body portion (e.g. and not the support portion) surrounds the connection region.

In some embodiments, the double layer is formed by folding a single tubular material portion one or more times to form a support portion and a body portion. Where, in some embodiments, a single end of the tubular material portion is connected at the connection region. In some embodiments, when in a crimped configuration, the device is unbent (e.g. around the connection region) and/or unfolded, the connection region, in some embodiments, laterally not being surrounded by body and/or support material.

In some embodiments, layers of a double-layered device are able to move with respect to each other. For example, in some embodiments, the body and support regions only being connected at a single connection region. For example, in some embodiments, the layers being formed using different tubular portions. A potential benefit being lower porosity (or higher coverage) for the device (e.g. portion/s of the device closing an entrance to an aneurysm) for a given crimped profile. A potential benefit being increase in reliability of expansion of the device after crimping.

An aspect of some embodiments of the invention relates to an intravascular device having a double layer, where the layers are connected to each other at a single connection region and/or where distal ends of the layers of the device are not connected. Where, in some embodiments, a single connection region is positioned at a proximal portion of the device. Where, in some embodiments, a first layer having a base and walls and a second layer having a base and walls, distal ends of the walls of the first layer and distal ends of the walls of the second layer are not connected to each other. In some embodiments, one or both of the first layer and the second layer are open where there is no structure closing and/or reducing an area of distal opening/s as defined by the walls of the layers.

An aspect of some embodiments of the invention relates to ends of portions of a mesh device.

Where, in some embodiments, distal edge portion/s of a device are free mesh e.g. which is un-connected to other element/s and/or extends to possibly contact tissue (e.g. as opposed to being tucked into the device volume and/or held by a connector). In some embodiments, ends of fibers of the mesh are blunted e.g. at least a portion of tips of ends of the mesh. A potential benefit being reduced risk of the ends causing trauma to tissue.

In some embodiments, blunting is by welding and/or coating the tips. In some embodiments, a portion of tips are blunted e.g. a circumferential portion e.g. outlying tips extending further than others from a body of the device.

In some embodiments, mesh filaments are interconnected. Potentially connecting (e.g. of crossing wires) of a mesh increases radial resistance and/or strength of the device and/or prevents delamination of the mesh (e.g. of the mesh weave). In some embodiments, connecting wires of a mesh is by welding and/or adhesive. In some embodiments, connecting is for an end region of the mesh e.g. a region more at risk of delamination. For example, for a region including a distal 0.1-3 mm, or 0.1-1 mm, or lower or higher or intermediate distances or ranges.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary System

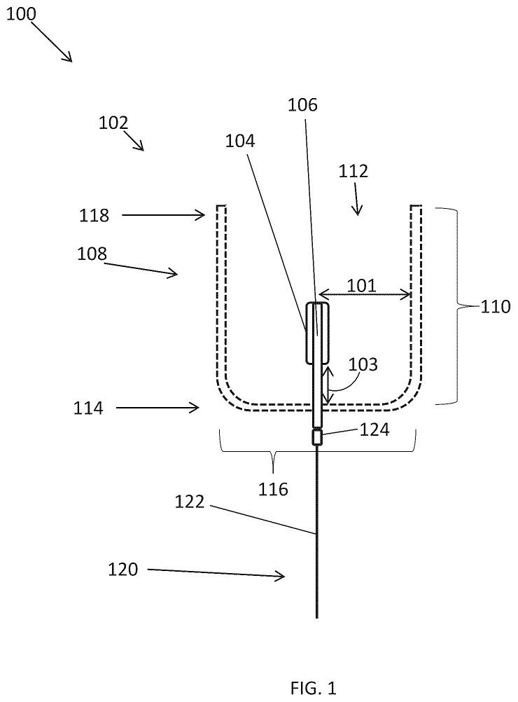

FIG. 1 is a simplified cross sectional schematic of an intravascular device system 100, according to some embodiments of the invention.

In some embodiments, system 100 includes an expandable intravascular device 102 (also herein termed “expandable device”, “intravascular device”).

In some embodiments, expandable device 102 has an expandable body 108. Where expandable body 108 has a distal end 118 and a proximal end 114.

In some embodiments, body 108 includes walls 110 and a base 116.

In an exemplary embodiment, expandable body 108 is at least partially formed by a mesh which, in FIG. 1 is indicated by dashed lines. In some embodiments, holes of the mesh are sized to restrict flow of blood through the mesh and/or to prevent escape of thrombi (e.g. over a certain size) from within a device lumen 112.

In some embodiments, one or more portion of the expandable body 108 is formed by a multi-layered (e.g. double layer) mesh, for example, illustrated in FIG. 1 by the double layer dashed line. Potential benefits of double-layering include higher resilience of the expandable body to collapse and/or increased reduction of movement of material (e.g. blood and/or thrombi) into and/or out of the aneurysm.

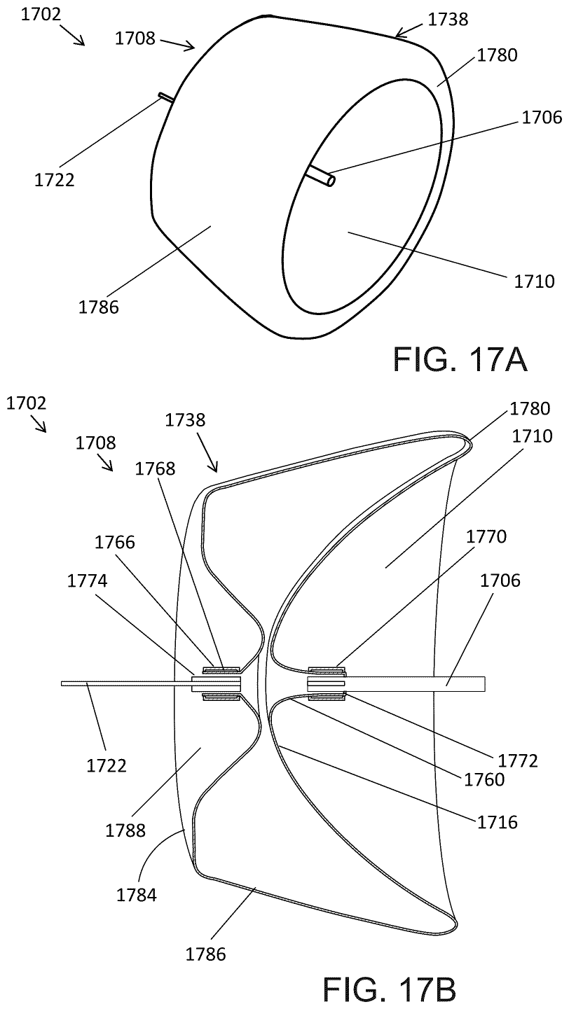

In some embodiments, a lumen 112 defined by walls 110 is closed, (e.g. at a proximal 114 end of the body) by base 116. Alternatively or additionally, a distal end 118 of lumen 112 is at least partially closed (e.g. as described and/or illustrated elsewhere in this document e.g. regarding FIG. 6, FIG. 7, FIG. 8, FIG. 10, FIGS. 17A-B).

In some embodiments, a radioactive source 104 (also herein referred to by the term “source”) is coupled to expandable device 102, for example, by a holder 106. Alternatively or additionally, in some embodiments, source 104 is directly attached to body 108. For example, by one or more of; welding mesh of body 108 to source 104, crimping body 108 to source 104, by gluing body 108 to source 104.

In some embodiments, holder 106 is elongate, extending from base 116 in a distal direction, for example, to position source 104 at a distance 103 of 0 to 10 mm, or 0.1-5 mm, or 0.1-3 mm, or 0.5-15 mm, or 1-1.5 mm or lower or higher or intermediate ranges or distances. In some embodiments, holder 106 and/or source 104 are positioned centrally with respect to base 116 and/or expandable body 108. For example, within a central 5-80%, or 5-50%, or 5-20%, or lower or higher or intermediate percentages or ranges of base 116. Where distance 101, for example, is 0.1 mm to 15 mm, or lower or higher or intermediate distances or ranges.

In some embodiments, expandable system 100 includes a delivery and/or positioning apparatus 120. In some embodiments, apparatus 120 includes an elongated element 122 which is coupled to expandable device 102. In some embodiments, elongated element 122 is a push-wire. Optionally, in some embodiments, a connector 124 connects push-wire 122 to expandable device 102. In an exemplary embodiment, push-wire 122 is connected to source mount 106 (directly and/or via a connector 124).

### Exemplary Methods

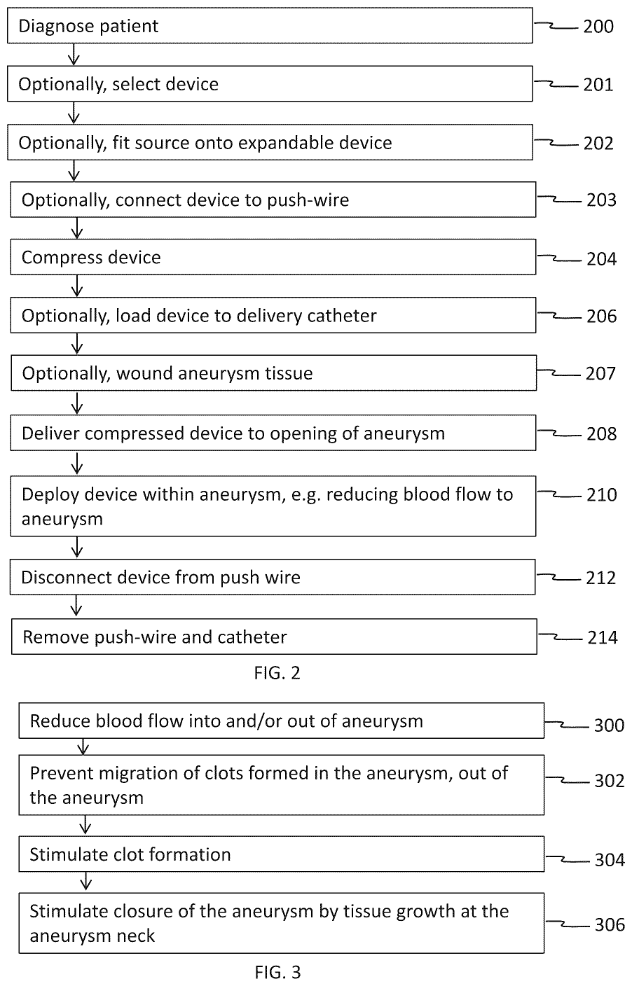

FIG. 2 is a flow chart of a method of treatment, according to some embodiments of the invention.

This flow chart generally describes a method of treatment of aneurysm, however, in some embodiments, relates to treatment of other tissue structure/s e.g. vascular structure/s and in an exemplary embodiment of treatment of the left atrial appendage (LAA).

At 200, in some embodiments, a patient is diagnosed, for example by a medical practitioner. In some embodiments, one or more type of measurement is collected from the patient. For example, imaging (e.g. one or more of x-ray, CT, MRI, ultrasound, nuclear imaging) of a site of vascular abnormality (e.g. aneurysm) and/or of a vascular structure (e.g. left atrial appendage). In some embodiments, measurements include a location and/or size and/or shape of the treatment site e.g. including an aneurysm for treatment (e.g. the left atrial appendage).

At 201, optionally, in some embodiments, an intravascular device is selected. Where, in some embodiments, the selection is based on the diagnosis e.g. including measurements. For example, in some embodiments, a size and/or shape of expandable body of the device is selected based on measurements (e.g. size and/or shape measurements) of the vascular region (e.g. aneurysm, e.g. left atrial appendage LAA) to be treated.

In some embodiments, a size and/or shape of an expandable body, for example, including size and/or shape of a base and/or walls of the device, is selected so that the expandable body covers an opening of the treatment site (e.g. opening of the aneurysm, e.g. LAA opening) e.g. covers at least 50-95%, or at least 80-90%, or at least 90%, or lower or higher or intermediate percentages or ranges of the opening area.

In some embodiments, a length of the device is selected so that the device, when expanded within the aneurysm (or LAA) extends from a top of the aneurysm (or LAA) to the aneurysm opening, the size and/or shape of the device holding a proximal portion (e.g. including the base of the expandable device) in position to close the opening of the aneurysm (or LAA).

In some embodiments, a size and/or shape of the expandable body in an expanded relaxed configuration is selected so that the expanded body within the aneurysm is larger than the aneurysm opening in one or more direction. Where, in some embodiments, over-sizing and/or elastic relaxation force are selected to provide sufficient force (e.g. outwards force from a center of the device e.g. radial force) between the device and aneurysm wall to hold the device in position within the aneurysm (or LAA).

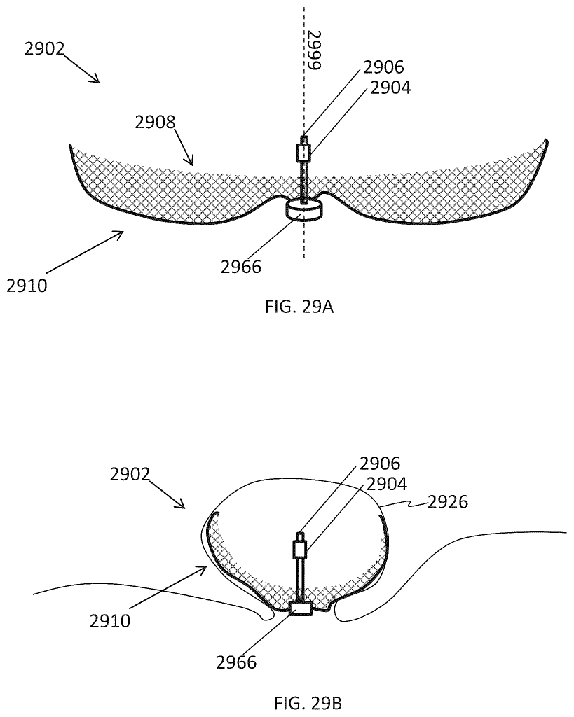

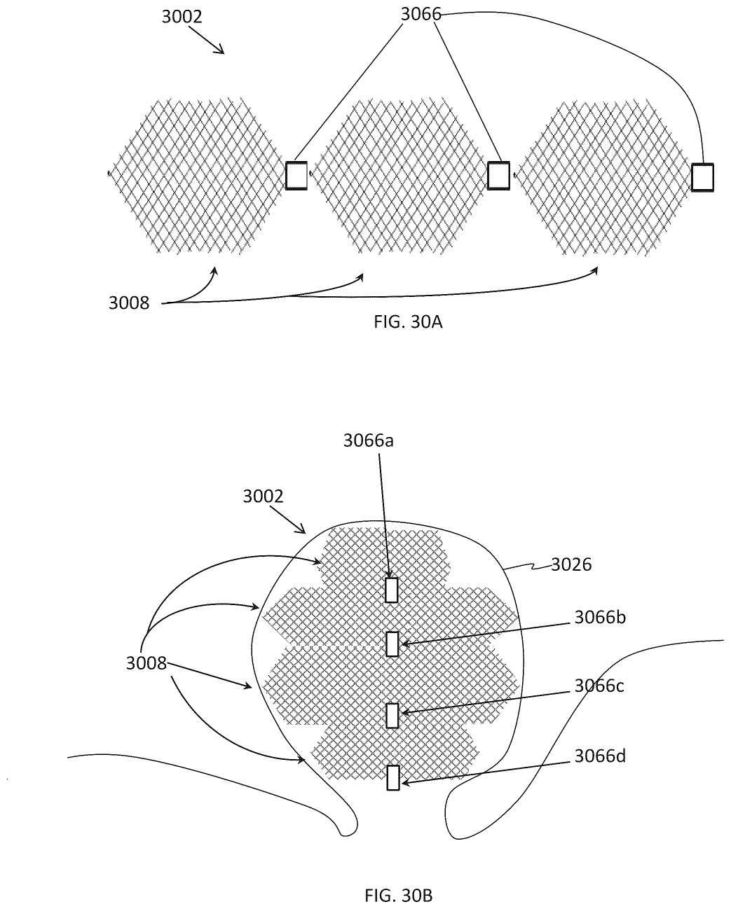

In some embodiments, a radioactive source of the intravascular device is selected. The source, for example, being selected based on the aneurysm size and/or shape and/or type. In some embodiments, for example, a larger dose and/or higher dose rate source is selected for a larger aneurysm. In some embodiments, the radioactive source includes more than one element, for example, where the radioactive elements are positioned at different regions of the expandable structure and/or aneurysm (e.g. including one or more feature as illustrated in and/or described regarding FIGS. 29A-B and/or FIGS. 30A-B). Where, in some embodiments, positioning of the radioactive elements is selected based on the size and/or shape and/or type of aneurysm (or LAA) to be treated.

In some embodiments, directional shielding of the source is selected. For example, to prevent irradiating particular tissue e.g. outside the aneurysm. For example, the optic nerve. In some embodiments, a shielding element is selected and optionally attached to the device and/or delivered through a catheter for positioning e.g. after the device is in position.

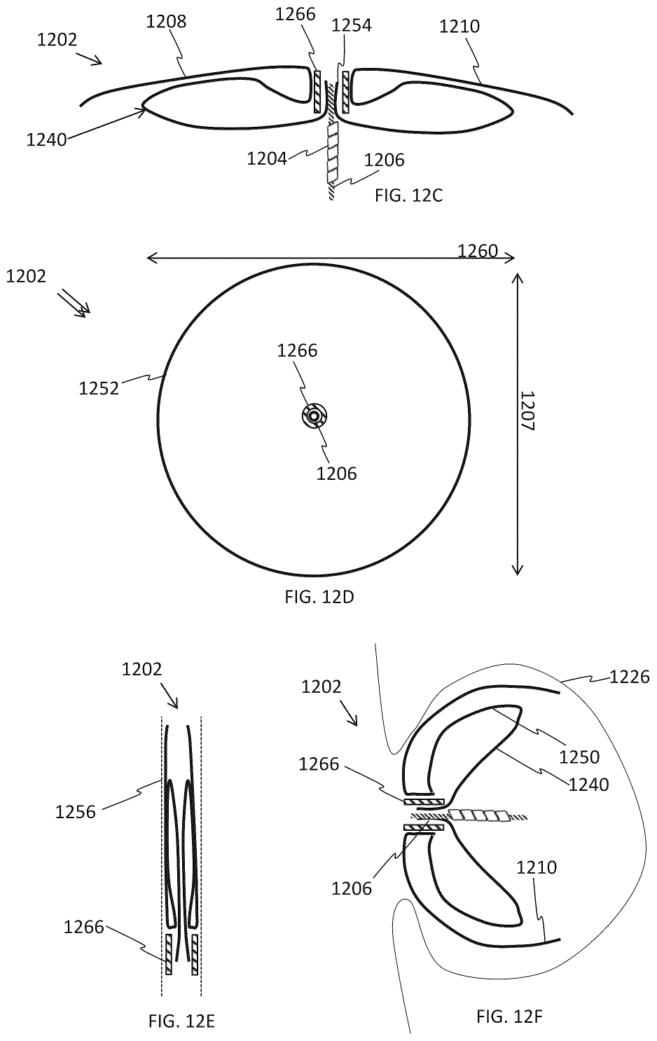

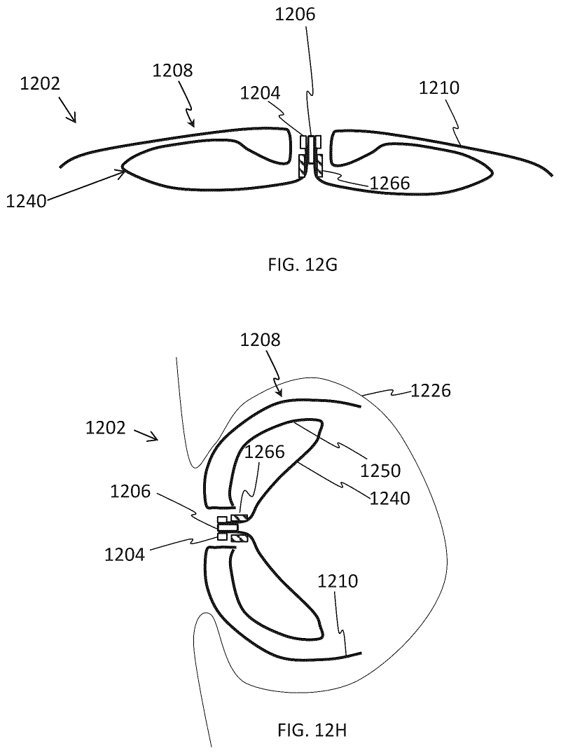

In some embodiments, selection is from a kit including a range of expandable bodies (e.g. as described regarding FIGS. 12A-F) and/or a range of radioactive sources. Where, in some embodiments, once a device is selected, the selected source is attached to the selected expandable body.

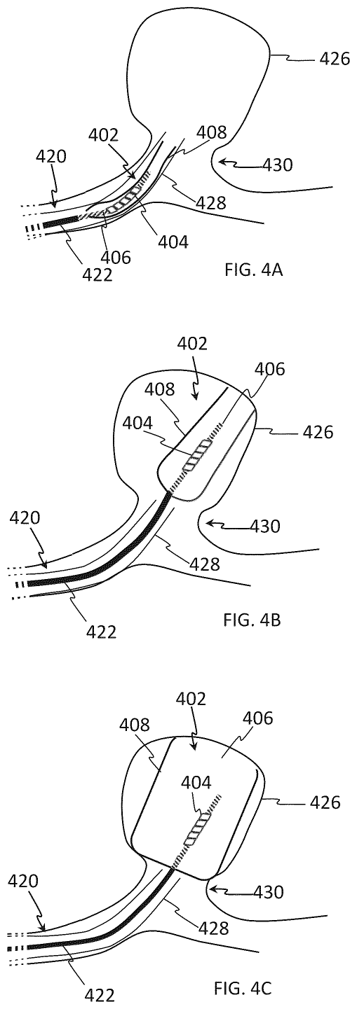

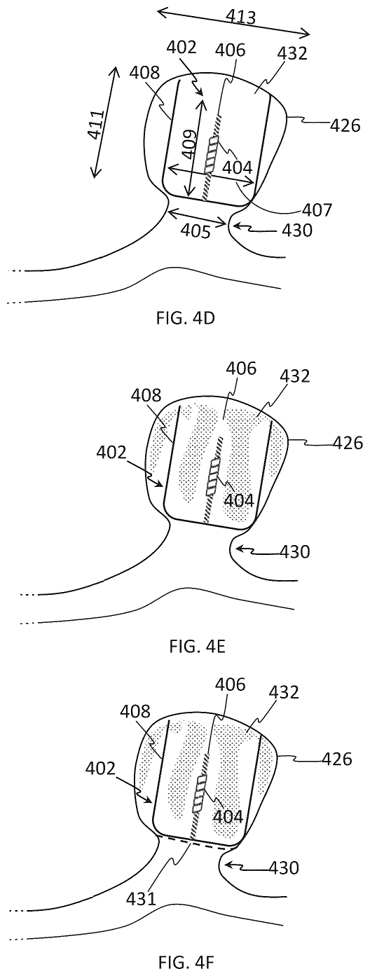

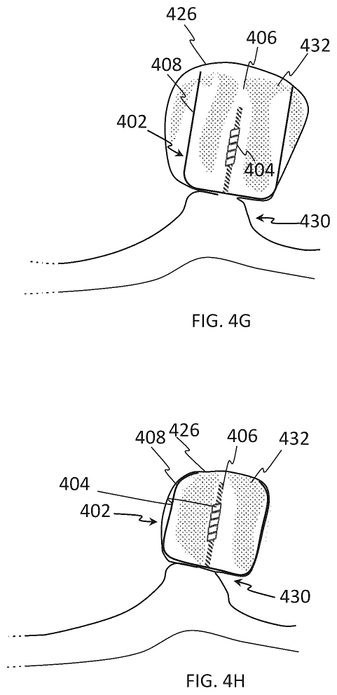

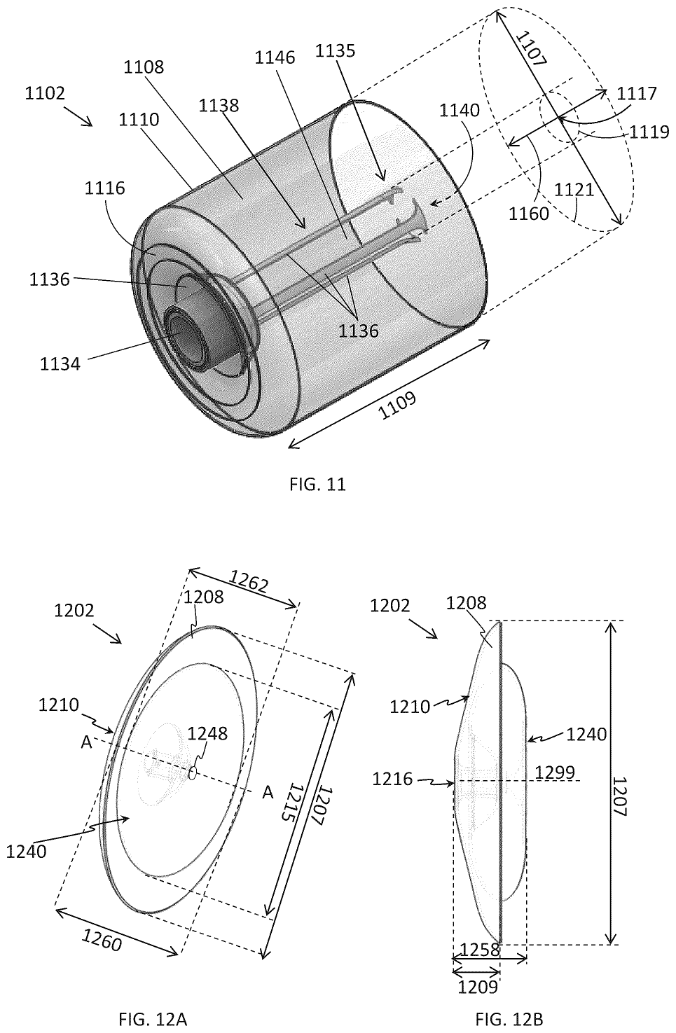

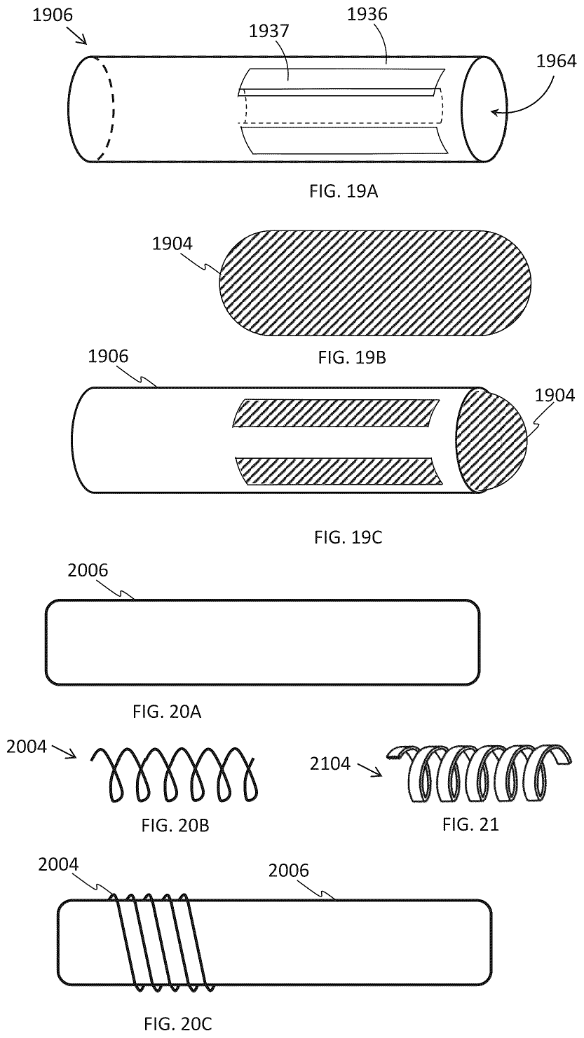

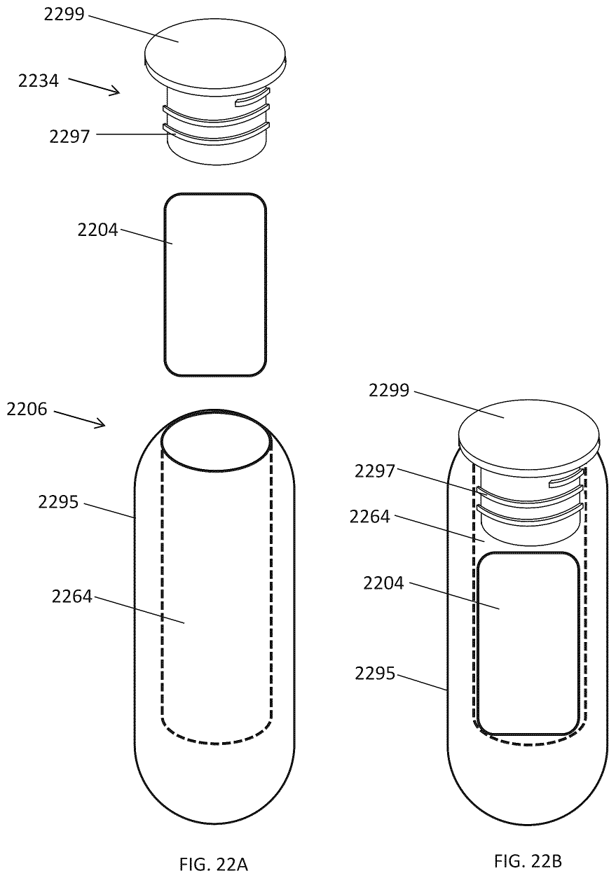

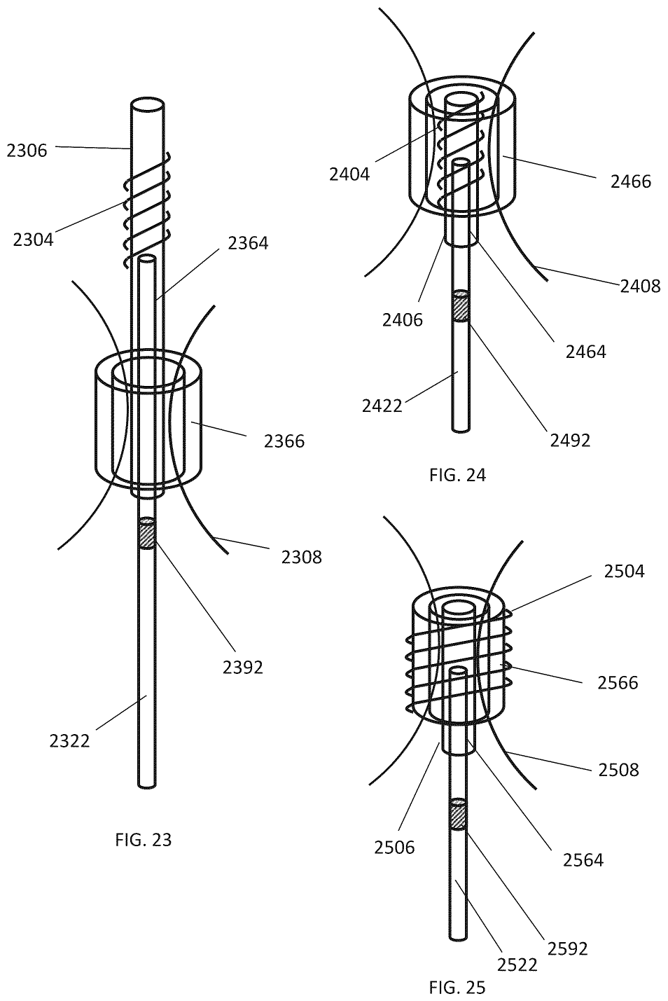

At 202, in some embodiments, a radioactive source is coupled to an expandable intravascular device.

Where the expandable device includes, for example, one or more feature as illustrated in and/or described regarding one or more of intravascular devices of FIGS. 1, 4A-H, 5-10, 11, 12A-H, 13A-C, 14A-C, 15A-B, 16A-B, 17A-B.

Where coupling of the radioactive source includes one or more feature as illustrated in and/or described regarding one or more of FIG. 11, FIGS. 19A-C, FIGS. 20A-C, FIG. 21, FIGS. 22A-B, and FIGS. 23-26.

In some embodiments, radioactive source/s are attached to the expandable device using a mounting tool. Where, in some embodiments, the tool allows a user to attach the radioactive source to expandable device without directly contact the source and/or device. Potentially maintaining sterility of the device and/or protecting the user from radiation exposure.

At 203, optionally, in some embodiments, the expandable device is connected to one or more portion of a delivery apparatus e.g. to a push-wire. Alternatively, in some embodiments, the expandable device is provided (e.g. prior to coupling of the radioactive source at step 200) pre-attached to a push-wire and/or delivery apparatus.

At 204, optionally, in some embodiments, the expandable device is compressed. Alternatively, in some embodiments, the expandable device is provided pre-compressed e.g. where coupling of the radioactive source is to a compressed device.

At 206, in some embodiments, the device is loaded into a delivery catheter. In some embodiments, steps 204 and 206 are combined where compressing is achieved, at least partially, positioning the expandable device within the delivery catheter. For example, by withdrawing the expandable device into the catheter and/or pulling the catheter over the expandable device.

At 207, optionally, in some embodiments, tissue wounding is performed at the aneurysm site. For example, wounding to the aneurysm neck and/or sac. In some embodiments, wounding is mechanical wounding. Alternatively or additionally, wounding is chemical e.g. via chemical agent/s. In an exemplary embodiment, the expandable device system is moved to inflict friction wounding on the aneurysm neck where the expandable device itself and/or push-wire and/or other portion/s of the expandable device delivery apparatus are moved against the aneurysm neck to wound it. Where the movement is backwards and forwards and/or rotational movement.

At 208, in some embodiments, the expandable device is delivered to an region of an aneurysm, for example, to an entrance of the aneurysm. Where, in some embodiments, the device is delivered in a contracted and/or crimped configuration within a catheter. In some embodiments, where the expandable device is elastically expandable, the device is restrained from expanding by walls of the catheter. For example, as part of a catheterization procedure. In some embodiments, delivery is assisted by imaging. In some embodiments, the expandable device is intravascularly delivered e.g. through a catheter e.g. movement of the device effected by pressure applied to a push-wire connected to the device.

At 210, in some embodiments, the expandable device delivered to the aneurysm and expanded within the aneurysm. Where, in some embodiments, delivery and expanding are simultaneous e.g. a self-expanding device being delivered into the aneurysm sac whilst exiting a delivery catheter. In some embodiments, a self-expanding device expands towards a relaxed configuration within the aneurysm. In some embodiments, the device expands to the relaxed confirmation. Alternatively in some embodiments, the aneurysm sac prevents the device from expanding fully and/or the device conforms at least partially to a shape of the aneurysm sac (e.g. the configuration of expandable device 402 FIG. 4H and/or of expandable device 1202 FIG. 12E). Where, for example, in some embodiments, elastic relaxation force of one or more portion of the expandable device is less than the resistance to expansion of portion/s of the aneurysm walls, shape of expandable device, at least partially conforming to a shape of the aneurysm.

Alternatively, in some embodiments, the device is delivered and then expanded afterwards e.g. where the device is mechanically expanded (e.g. balloon expansion) and/or where a restraining element (e.g. catheter) is withdrawn after the device is within the aneurysm.

At 212, in some embodiments, delivery apparatus (e.g. the push-wire) is disconnected from the expandable device. Where, in some embodiments, detachment is by one or more of mechanical detachment, electrolytic detachment and electrothermal detachment.

For example, where disconnecting includes one or more feature as illustrated in and/or described regarding detachment region 2392 FIG. 23.

At 214, in some embodiments, the delivery apparatus, for example, including the push-wire and/or catheter are retracted e.g. from the subject's body.

FIG. 3 is a flow chart of a method of treatment, according to some embodiments of the invention.

At 300, in some embodiments, blood flow into and/or out of an aneurysm is reduced. For example, mechanically, by a mesh at least partially blocking an entrance to the aneurysm.

At 302, in some embodiments, thrombi formation within the aneurysm sac is stimulated e.g. by exposure of the aneurysm lumen to low level radiation e.g. the radiation levels including one or more feature as described in the section of this document entitled “Exemplary radiation levels”.

At 304, in some embodiments, migration of formed clots (e.g. above a certain size) within the aneurysm to outside the aneurysm is prevented. For example by mesh blocking a passage of thrombi within the aneurysm and/or within a volume defined by the mesh structure to a lumen of a blood vessel to which the aneurysm is a deformity.

At 306, in some embodiments, closure of the entrance to the aneurysm is stimulated.

Optionally, in some embodiments, stimulation includes wounding (e.g. mechanical and/or chemical) of tissue of the entrance to the aneurysm (also termed in this document “aneurysm neck”).

In some embodiments, stimulation includes exposure to low level ionizing radiation. Which, in some embodiments, stimulates closure of the aneurysm neck by enothelization of the mesh over the aneurysm neck followed by neointimal formation.

FIGS. 4A-H are simplified schematic cross sections showing treatment of an exemplary aneurysm 426, according to some embodiments of the invention.

Referring to FIG. 4A, in some embodiments, an expandable device 402 is delivered in a collapsed configuration by a delivery apparatus 420 to an opening of aneurysm 426. Where, in some embodiments, delivery apparatus 420 includes a catheter 428 and expandable device 402 is delivered through catheter 428. In some embodiments, when collapsed (e.g. within catheter 428) an expandable body 408 of expandable device 402 is collapsed against a mount 406 which hosts a radioactive source 402.

Referring to FIG. 4B, in some embodiments, expandable device 402 is delivered to aneurysm 426. Referring to FIG. 4C, in some embodiments, expandable device 402 is expanded within aneurysm 426. Where delivery and/or expanding includes one or more feature as illustrated in and/or described regarding step 210 FIG. 2.

In some embodiments, once expanded, the expandable device 402 is moved e.g. using push-wire 422. For example, to wound tissue e.g. as described in step FIG. 2. For example, to position and/or reposition the expandable device within aneurysm 426.

Referring to FIG. 4D, in some embodiments, once the device is positioned within aneurysm 426, delivery apparatus 420 is removed.

In some embodiments, expandable device 402 is configured to hold a base of device 402 in position (e.g. touching tissue around the opening) to close aneurysm opening at aneurysm neck 430. In some embodiments, in a proximal-distal direction a length 409 of expandable device is selected, with respect to a length 411 of the aneurysm to hold the device in position. In some embodiments, expandable device 402 is selected so that, at least in a length dimension, the expandable device, in a relaxed configuration, is larger than the aneurysm length 411 so that the elastic expansion force of device 402 on aneurysm 426 walls holds device in position. In some embodiments, the force is not sufficient to deform the walls of the aneurysm and/or is not sufficient to weaken the walls of the aneurysm. Where, for example, in some embodiments, for one or more direction (e.g. at least length e.g. all directions) the relaxed device dimension in that direction is at most 5-50%, or 5-20%, or 5-10%, or lower or higher or intermediate percentages or ranges larger than a maximum dimension and/or average dimension of the aneurysm (e.g. in that direction).

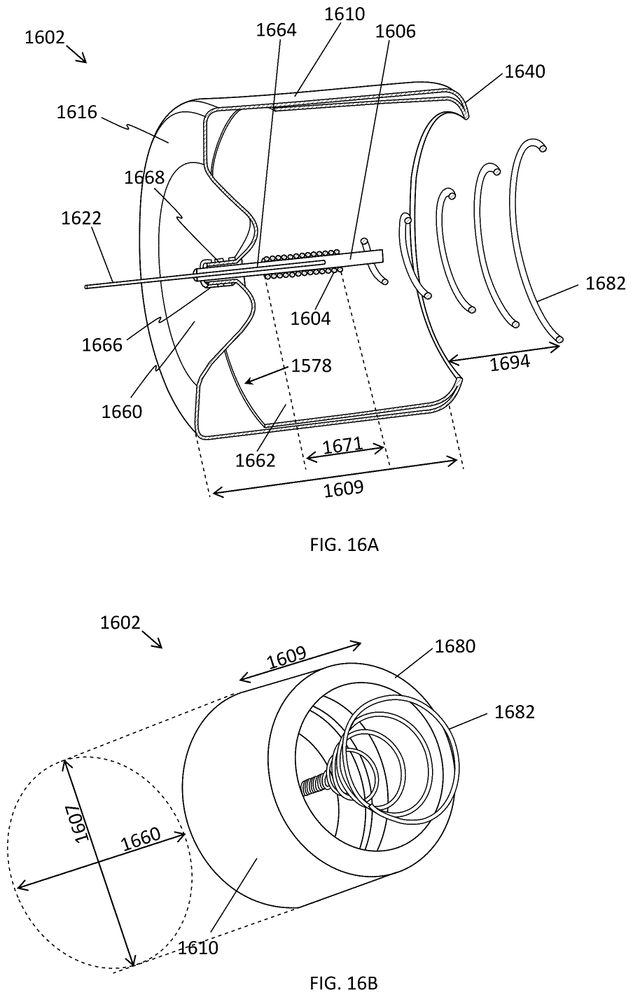

In some embodiments, expandable device 402 includes an additional expandable support which, in some embodiments, extends from expandable body 408 e.g. to contact aneurysm 426. For example, including one or more feature as illustrated in and/or described regarding support 1682 FIGS. 16A-B.

In some embodiments, expandable device 402 expands in width 407 and/or is sized and/or shaped to hold device 402 in position at the aneurysm opening. Where, in some embodiments, the expandable device does not contact a roof of the aneurysm and/or does not fill a length 411 of the aneurysm.

In some embodiments, an aneurysm neck width 405 is 0.5-20 mm, or 1-15 mm, or 2-13 mm or lower or higher or intermediate widths or ranges.

In some embodiments, an average and/or maximal dimension of the aneurysm e.g. height 411 and/or width 413 is 0.5-20 mm, or 2-15 mm, or 2-14 mm, or 2-13 mm, or lower or higher or intermediate dimensions or ranges. In some embodiments, (e.g. in a relaxed configuration) a width 407 and/or length 409 of expandable body 408 is 1-20 mm, or 2-15 mm, or 3-14 mm, or lower or higher or intermediate dimensions or ranges.

Referring to FIG. 4E, in some embodiments, thrombi 432 form (e.g. according to one or more feature as described in and/or illustrated regarding step 304 FIG. 3) within expandable device 402 and/or between expandable device 402 and aneurysm 426 walls.

Referring to FIG. 4F, in some embodiments, tissue growth (e.g. according to one or more feature as described in and/or illustrated regarding step 306 FIG. 3) at aneurysm neck 430 acts to close aneurysm 426. Where, in some embodiments, tissue first begins to grow onto mesh of expandable device 402 e.g. as illustrated by dashed line 431. After which, in some embodiments, tissue continues to grow to close the entrance to the neck. For example, as illustrated in FIG. 4G. In some embodiments, closure of the neck is achieved while expandable device 402 remains expanded within the aneurysm.

Alternatively, in some embodiments, referring to FIG. 4G, aneurysm 426 sac begins to contract, collapsing onto expandable device 402. In some embodiments, aneurysm 426 continues to contract. In some embodiments, contraction of aneurysm partial collapses expandable device 402 e.g. as illustrated in FIG. 4H.

### Exemplary Expandable Devices

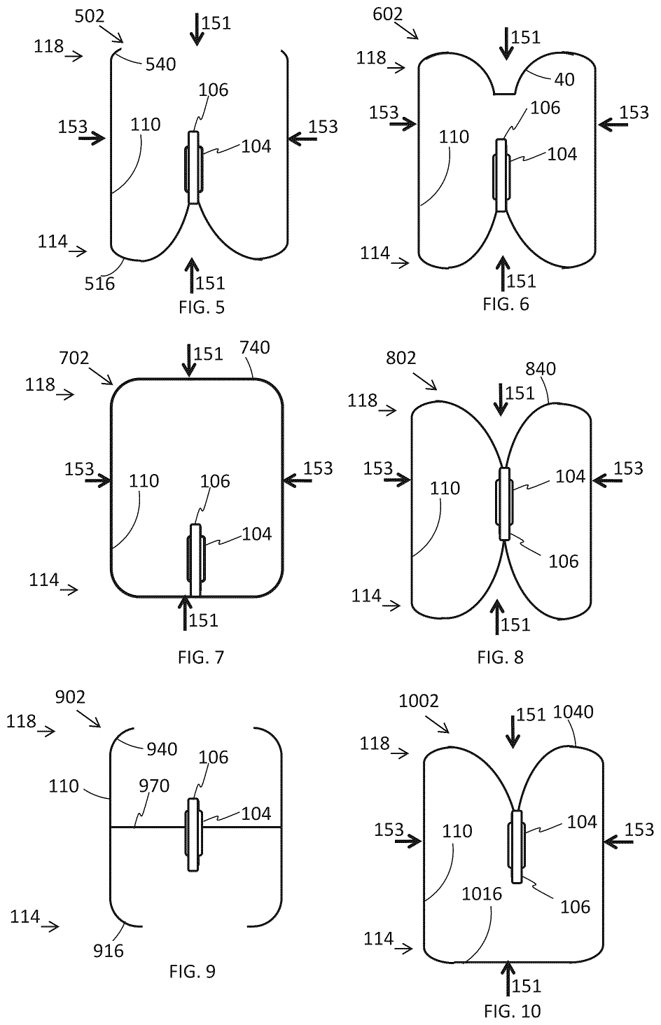

FIGS. 5-10 are simplified schematic cross sectional views of exemplary expandable devices.

In some embodiments, each device 502, 602, 702, 802, 902, and 1002, has a proximal end 114 and a distal end 118 and includes tubular walls 110, a source 104 which, in some embodiments, is attached to a body of the expandable device via a mount 106.

All of FIGS. 5-10 illustrate exemplary embodiments where, for at least the cross section illustrated in FIGS. 5-10, source 104 is located at a central region of the expandable device in a direction perpendicular to a proximal-distal direction. In some embodiments, source is located at a central region of the device is all directions perpendicular to the proximal-distal direction.

FIG. 5, FIG. 6, and FIGS. 8-10 illustrate exemplary embodiments where source 104 is positioned within a central region of the expandable device in a proximal-distal direction.

FIGS. 5-9 illustrate exemplary embodiments where source 104 and/or holder 106 are attached to a base of the device; bases 516, 616, 716, 816.

FIG. 5, FIG. 6, and FIG. 8 illustrate exemplary embodiments, where source 104 and/or holder 106 are attached to a recessed device base 516, 616, and 816 respectively.

FIGS. 6-8 and FIG. 10 illustrate exemplary embodiments where the device includes a closed top 640, 740, 840, 1040.

FIG. 5 and FIG. 9 illustrate exemplary embodiments including a top with an opening 540, 940.

FIG. 6, FIG. 8 and FIG. 10 illustrate exemplary embodiments, where tops 640, 840 and 1040 are attached to source 104 and/or holder 106.

In some embodiments, expandable devices experience (e.g. at a treatment site e.g. within an aneurysm) to forces 151, 151. For example, compressive forces 151 in a proximal-distal direction and/or forces 153 in a direction perpendicular to the proximal-distal direction (e.g. axial direction).

In some embodiments, a shape of a recessed portion (e.g. base and/or top) provides structural resilience to contraction and/or collapse in the proximal-distal direction e.g. embodiments illustrated in FIGS. 6, FIG. 8, and FIG. 10.

In some embodiments, closure, even if only partial, of a top (or base) provides increased structural resilience in the proximal-distal direction e.g. as illustrated in all of FIGS. 5-10 where, for example, partial closure e.g. as illustrated for top 540 FIGS. 5 and/or 940 top, base 916 FIG. 9.

FIG. 5 illustrates an exemplary embodiment where base 516 is partially recessed within walls 510 and where device 502 lacks a top (or where the top partially covers a lumen defined by walls 110.

FIG. 6 illustrates an exemplary embodiment where both base 616 and top 640 are partially recessed within walls 610.

FIG. 7 illustrates an exemplary embodiment including both a closed base 716 and a closed top 740 where neither base 716 nor top 740 are recessed. In some embodiments, source 104 and/or holder 106 are disposed at a proximal end of expandable device 702.

FIG. 8 illustrates an exemplary embodiment where both a base 816 and a top 840 are closed and/or recessed.

FIG. 9 illustrates an exemplary embodiment where both a base 916 and the top 940 are open and a support structure 970 connects source 104 and/or holder 106 to walls 110.

FIG. 10 illustrates an exemplary embodiment where source 104 and/or holder 106 are attached to a top 1040 (and not to a base 1016). Where, in some embodiments, both base 1016 and top 1040 are closed. In some embodiments, top 1040 is recessed and base 1016 is not recessed. Alternatively both top 1040 and base 1016 are recessed, e.g. without base 1016 being attached to source 104 and/or support 106.

FIG. 11 is a simplified view of an expandable device 1102, according to some embodiments of the invention.

In some embodiments, expandable device 1102 includes an expandable body 1108 with walls 1110 and a base 1116. In some embodiments, one or more portion of body 1108 is formed from mesh e.g. flow diverter braided mesh (e.g. including one or more feature as described in the section of this document entitled “Exemplary expandable body materials”). Optionally, in some embodiments, body 1108 includes a top 1140.

In some embodiments, arrow 1109 illustrates a proximal distal direction for device 1102 where, in some embodiments, base 1116 is proximal of top 1140.

In some embodiments, length 1109, width 1107 and/or depth 1160 (where, in some embodiments, cross section 1121 is symmetrical) are 1-20 mm, or 2-15 mm, or 3-14 mm, or lower or higher or intermediate dimensions or ranges. In some embodiments, walls 1110 form a tubular structure. Where, in some embodiments, a lumen enclosed by the walls maintains a constant cross section along a length 1109 of walls 1110. In some embodiments, base 1116 closes a proximal end of walls 1110. In some embodiments, length 1109 is 1-20 mm, or 2-10 mm, or 2-15 mm, or 3-14 mm, or lower or higher or intermediate dimensions or ranges.

In some embodiments, a length 1109 to width 1107 and/or depth 1160 ratio is 1:1 to 1:10, or 1:1 to 1:5, or lower or higher or intermediate ratios or ranges.

In some embodiments, expandable device 1102 includes a source holder 1138 connected to expandable body 1108 e.g. at base 1116. For example, at a central region of base, for example, where in some embodiments, holder 1136 and/or a holder cross section 1119 center 1117 is disposed within a central region of a cross section 1121 of expandable body 1108 where the cross section is taken parallel to base 1116 and/or perpendicular to walls 1110. For example, where holder cross section 1119 and/or center 1117 is in a central region of expandable body cross section 1121 e.g. where centre 1117 is disposed within a central region of expandable body cross section 1121 width 1107 and/or depth 1160. Where, in some embodiments a central region is defined as being a central 5-80%, or 5-50%, or 5-20%, or lower or higher or intermediate percentages or ranges of a length and/or cross section area.

In some embodiments, holder includes a lumen 1146 sized and/or shaped to receive a radioactive source. In some embodiments, a holder cross section average and/or maximum cross sectional dimension 1109 is 0.05-1 mm, or 0.05-0.5 mm, or 0.1-0.4 mm or lower or higher or intermediate ranges or dimensions. In some embodiments, holder 1106 extends within a majority of length 1109 of device e.g. 50-95%, or 50-80%, or lower or higher or intermediate percentages or ranges, of device length. Alternatively, in some embodiments, holder is short with respect to length 1109, with a length of at most 50%, or 5-30%, or lower or higher or intermediate ranges of percentages of a device length 1109. In some embodiments, a long holder holds a short (e.g. less than 50% of the holder lumen's length) source, potentially enabling selection of positioning within the holder of the source and/or enabling loading of the holder with more than one source e.g. as illustrated in FIG. 27 where holder 2706 holds two sources 2704a, 2704b.

In some embodiments, lumen 1146 is defined by a plurality of arms 1136. In some embodiments, arms 1136 are connected at a proximal end by a holder base 1136. In some embodiments, holder base 1136 is connected to expandable body 1108 base 1116.

In some embodiments, portion/s 1134, 1136 form a cap for holder 1138. In some embodiments portion 1134 is an attachment for a push-wire (not illustrated) to expandable body 1108. In some embodiments, attachment 1134 is a screw attachment, including threading on connector 1134 and/or portion 1136. Where, in some embodiments, to detach a push-wire, portion 1136 is unscrewed from portion 1134. Other attachment mechanisms (e.g. as described elsewhere in this document) between portions 1134, 1136 are envisioned and encompassed.

In some embodiments, holder 1138 is connected to expandable body 1108 by a connector which, in some embodiments, has a ring shape and/or is optionally radiopaque. Where, in some embodiments, connection is by one or more of crimping, welding, and quenching.

Optionally, in some embodiments, arms 1136 are connected in one or more additional position distal of holder base 1136 (not illustrated) additional connection/s potentially reinforcing the holder.

In an exemplary embodiment, distal ends 1135 of arms 1136 are shaped to prevent a source from exiting lumen 1146 distally. For example, in some embodiments, distal ends 1135 of arms 1136 curve to reduce a cross section of lumen 1146 e.g. at a distal end of lumen 1146. Alternatively, or additionally, in some embodiments, holder includes a base attached to the arms 1136 e.g. at distal ends 1135 of the arms.

Optionally, in some embodiments, holder 1138 includes a cap 1134 which, in some embodiments, is sized and/or shaped to prevent a radioactive source within lumen 1146 from falling out. In some embodiments, cap 1134 closes an end of lumen 1146. Alternatively, in some embodiments, cap 1134 constricts the opening of lumen 1146 to a size smaller than allows passage of the radioactive source. In some embodiments, cap 1134 connects to holder by a screw mechanism. For example, where cap 1134 and/or holder base 1136 include threading. In some embodiments, cap 1134 and/or holder 1138 include one or more feature of cap 2499 and/or holder 2406 as described regarding and/or illustrated in FIG. 24.

Optionally, in some embodiments, once the radiative source is positioned within lumen 1146 it is fixed into position, for example by deforming one or more arms 1136 e.g. towards the radioactive source. In embodiments where the arms are deformed, cap 1134 is optional.

FIGS. 12A-F are simplified schematic views of an expandable device 1202, according to some embodiments of the invention.

FIG. 12A is a simplified schematic view of an expandable device 1202, from a distal direction, according to some embodiments of the invention.

FIG. 12B is a simplified schematic side view of an expandable device 1202, according to some embodiments of the invention.

In some embodiments, FIGS. 12A-D illustrate expandable device 1202 in a relaxed configuration e.g. without externally applied forces on the expandable device 1202.

In some embodiments, expandable device 1202 has an expandable body 1208 which includes walls 1210 and a base 1216. Walls 1210 and base 1216, in some embodiments, form a flattened cup shape, for example, when device 1202 is in a relaxed expanded configuration. Where, in some embodiments, a width 1207 and/or height 1260 of the shape formed by walls 1210 and base 1216 is at least 1.5 times, or at least double, or 1.5-10 times or 3-10 times or, 3-7 times or about 5 times a thickness 1209 of the shape.

In an exemplary embodiment, a kit of expandable bodies (e.g. where one of the bodies is selected for treatment of a particular aneurysm e.g. based on size of the aneurysm) includes devices with width of 5 mm, 7 mm, 9 mm, 11 mm and 14 mm (and/or height 1260, for example, in embodiments where the device is rotationally symmetric about a central distal proximal axis 1299). Where, in some embodiments, for the kit, thickness is 3-7 times the height and/or of width.

In some embodiments, the device is symmetrical about one or more axis (e.g. when in a relaxed configuration). For example, in some embodiments, width 1260 and height 1207 of expandable body 1208 are the same, e.g. within 10%, or 5%, or 2%, or lower, or higher, or intermediate percentages of each other. In an exemplary embodiment maximum and/or average width 1260 and/or height 1207 are 1-20 mm, or 2-20 mm, or 4-15 mm, or lower or higher or intermediate dimensions or ranges. In some embodiments, expandable device includes one or more support 1240. In some embodiments, support 1240 reinforces walls 1210 and/or supports a radioactive source (radioactive source not illustrated in FIGS. 12A-B).

In some embodiments, support 1240 is large with respect to walls 1210, for example, with a maximal and/or average cross sectional dimension (and/or height 1215 and/or width 1262) which are 40-110%, or 50-100%, or 60-90%, or lower or higher or intermediate percentages or ranges of the equivalent dimension for walls 1210. Where a large support potentially strengthening device 1202 potentially preventing collapse of the device and/or providing strong blockage of flow of fluid into and/or out of aneurysm.

Alternatively, in some embodiments, support 1240 is smaller, for example, with a maximal and/or average cross sectional dimension (and/or height 1215 and/or width 1262) which are 1-20%, or 1-10% or lower or higher or intermediate percentages or ranges of the equivalent dimension for walls 1210. Where a smaller support potentially facilitates crimping and/or deployment of device 1202.

In some embodiments, the radioactive source is enclosed by expandable structure 1208 where, for example, the source is enclosed proximally by base 1216 and walls 1210 and is enclosed distally by support structure 1240 (except for, in some embodiments, opening 1248 in support structure 1240). In some embodiments, with respect to description elsewhere in this body support structure 1240 and/or a distal surface of support structure 1240 is considered to be a top to the device.

In some embodiments, opening 1248 provides access to an inner volume of expandable device 1202, e.g. for affixing the radioactive source within expandable body 1208. In some embodiments, opening 1248 has small area, for example, at most 1-10%, or 1-5%, or lower, or higher, or intermediate percentages or ranges of a cross section of maximal extent of walls 1210 (e.g. as illustrated by dimension 1260, 1207).

FIG. 12C is a simplified schematic cross sectional view of an expandable device 1202, according to some embodiments of the invention.

In some embodiments, FIG. 12C shows a cross section taken at a central axis of expandable device 1202 e.g. along line A-A marked in FIG. 12A.

FIG. 12C, in some embodiments, illustrates a radioactive source attached 1204 to expandable body 1208 by a holder 1206.

In some embodiments, source 1204 is attached to holder 1206 as described and/or illustrated regarding one or more of FIGS. 19A-C, FIGS. 20A-C, FIG. 21, FIGS. 22A-B, FIGS. 23-26.

In an exemplary embodiment, source 1204 is attached to holder 1206 by elastically deforming the source 1204 in attaching the source to the holder, such that elastic relaxation force of the source holds it onto the holder. Where, for example, in some embodiments, the source is wound around the holder 1206 one or more times e.g. is a coil shape. Where, in some embodiments, a wound on and/or wrapped around source is optionally elastically deformed in the attachment e.g. a relaxed lumen of the source is smaller than an external dimension of the holder 1206 where the source is attached.

In some embodiments, holder 1206 is attached to expandable body by a connector 1266. In some embodiments, connector 1266 has a ring shape. Optionally, in some embodiments, connector 1266 includes radiopaque material. In some embodiments, holder 1206 and source are not illustrated in FIGS. 12A-B. In some embodiments, holder 1206 extends away (e.g. distally) from expandable body. A potential advantage of which is the ability to position source 1204 within a central region of the aneurysm e.g. as illustrated in FIG. 12F. A potential disadvantage of an extending holder 1206 is increased risk of rupture of aneurysm 1226 by the holder e.g. during delivery and/or deployment of device 1202.

In some embodiments, support structure 1240 includes more than one layer. Where, in an exemplary embodiment, support structure 1240 is a loop extending from a central region of device 1202. In some embodiments, support structure 1240 is connected to the radioactive source 1204, for example, at a central region (e.g. central 20%) of the support structure.

In some embodiments, support structure 1240 provides additional layers (e.g. of mesh) providing blocking of the aneurysm opening. In some embodiments, support structure 1240 potentially provides additional mechanical resistance to collapse of the device.

FIG. 12D is a simplified schematic top view, from a proximal direction, of an expandable device 1202, according to some embodiments of the invention.

FIG. 12E is a simplified schematic cross section of a collapsed expandable device 1202, according to some embodiments of the invention.

In some embodiments, for example, as illustrated in and/or described regarding FIG. 2, expandable device 1202 is delivered to a treatment site in a collapsed configuration. Where, in some embodiments, the device is collapsed by being inserted into a catheter 1256. In some embodiments, the device is delivered through catheter 1256.

In some embodiments, in a collapsed configuration within a catheter 1256, the expandable body is extended so that portion/s do not overlap, the expandable body forming a single layer tubular structure within the catheter.

FIG. 12F is a simplified schematic cross section of an expandable device 1202 within an aneurysm 1226, according to some embodiments of the invention.

In some embodiments, walls 1210 and/or support structure 1240 conform, at least partially to a shape of aneurysm 1226. For example, walls 1210 and/or support structure 1240 bending away from a relaxed configuration towards a central region of the device and/or towards the source 1204. In some embodiments, expandable device 1202 bends in to a hemispherical shape.

A potential advantage of an expandable device 1202 which at least partially conforms to the aneurysm is the ability to deliver the device to the aneurysm from a variety of different angles and have the device walls be able to block the opening of the aneurysm.

Expandable device 1202, in some embodiments, presents a gentle curve at a proximal portion of the device, at least when the device is deployed within an aneurysm, (e.g. curve provided by base 1216 and/or walls 1210). In some embodiments, the gentle curve shape is, additionally or alternatively, provided by bending of walls 1210 as they conform to the aneurysm shape. A potential benefit of the gentle curve is that for a range of different rotational orientations of the expandable device 1202 within aneurysm 1226, the surface of the device is non-disruptive to flow of fluid (e.g. associated with reduced risk of clotting) through the vessel to which the aneurysm is a deformity.

In some embodiments, (not illustrated), walls 1210 and optionally base 1216 are formed of a double layer mesh, potentially providing increased reduction of flow between aneurysm 1226 inner volume and the vessel to which aneurysm 1226 is a deformity and/or increased scaffolding potentially increasing rate of growth of tissue to close the opening of the aneurysm.

FIG. 12G is a simplified schematic cross sectional view of an expandable device 1202, according to some embodiments of the invention.

In some embodiments, FIG. 12G shows a cross section taken at a central axis of expandable device 1202 e.g. along line A-A marked in FIG. 12A.

FIG. 12H is a simplified schematic cross section of an expandable device 1202 within an aneurysm 1226, according to some embodiments of the invention.

In some embodiments, the device as illustrated in and/or described in FIGS. 12A-F includes an alternative connection of a source 1204 to an expandable body 1208 (e.g. alternative than that illustrated in FIG. 12C and FIG. 12F).

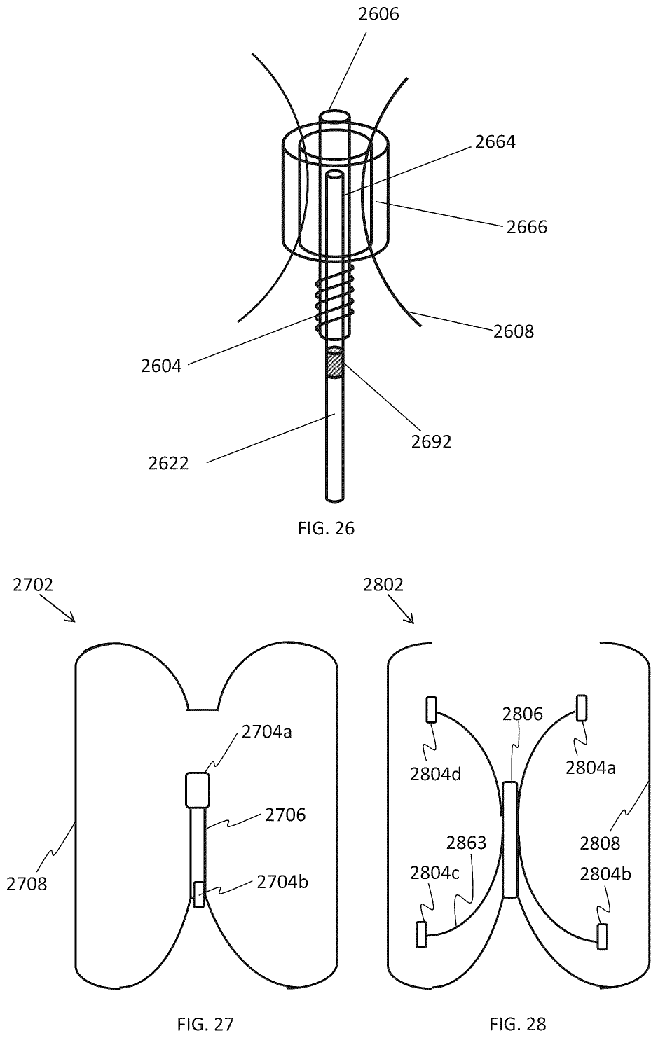

In some embodiments, source 1204 is disposed proximally of a connector 1266 (e.g. proximal position of a source including one or more feature illustrated in and/or described regarding source 2604 and/or holder 2606, and/or connector 2666, FIG. 26).

In an exemplary embodiment, source 1204 is attached to holder 1206 by elastically deforming the source 1204 in attaching the source to the holder, such that elastic relaxation force of the source holds it onto the holder. In some embodiments, the source is wound around the holder 1206 one or more times e.g. is a coil shape. Where, in some embodiments, a wound on and/or wrapped around source is optionally elastically deformed in the attachment e.g. a relaxed lumen of the source is smaller than an external dimension of the holder 1206 where the source is attached.

In some embodiments, connector 1266 holds mesh portions together without a holder therebetween e.g. without support 1206 therebetween (e.g. in some embodiments, connector 1266 is directly connected e.g. welding of the mesh and/or quenching of the connector onto the mesh). In some embodiments, source 1204 is a coil and/or ring directly attached e.g. crimped onto mesh of expandable body 1208 e.g. without support 1206 therebetween.

Where neither connector 1266 nor source 1204 hold mesh onto holder 1206, holder is optional. In some embodiments, source 1204 itself connects the mesh where both holder 1206 and connector 1266 are optional.

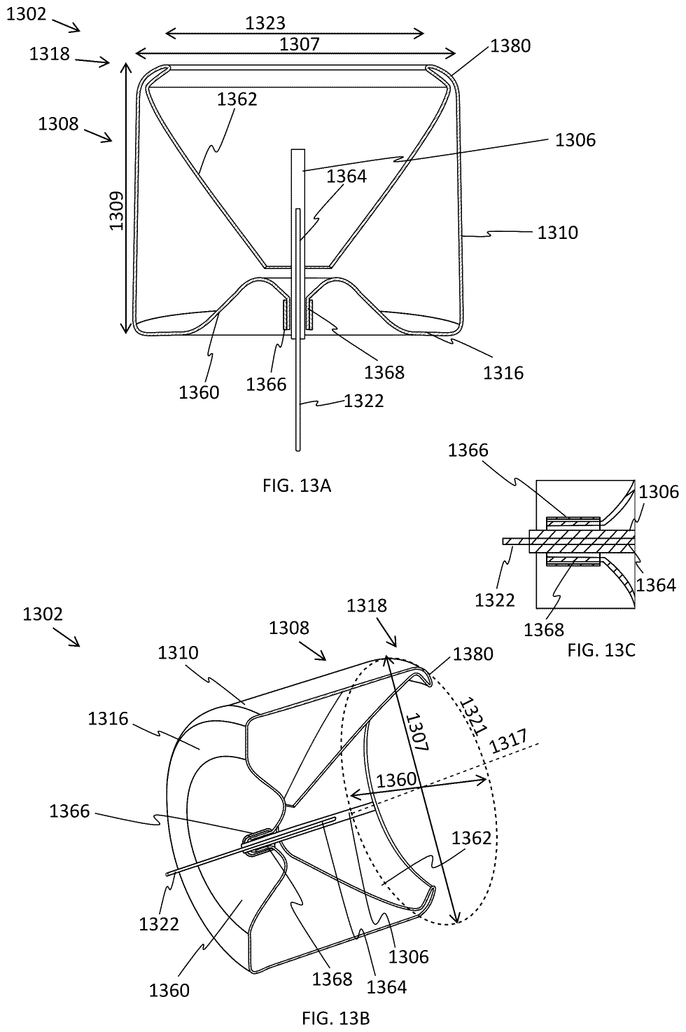

FIGS. 13A-B are simplified schematic sectional views of an expandable device 1302, according to some embodiments of the invention.

FIG. 13C is a simplified schematic cross sectional view of a portion of an expandable device 1302, according to some embodiments of the invention.

In some embodiments, FIGS. 13A-B and FIG. 13C show the same expandable device 1302.

In some embodiments, expandable device 1302 includes an expandable body 1308 having walls 1310 and a base 1316. In some embodiments, a shape of walls 1310 includes one or more feature as illustrated and/or described regarding walls 1110 FIG. 11.

In some embodiments, base 1316 includes a recessed central portion 1360. A potential benefit of which is recessing of connection/s (e.g. of base 1368 and/or of holder 1306 and/or of push-wire 1322) away from flow within the lumen to which the aneurysm is a deformity. In some embodiments, recessed portion 1360 is a small portion of an area of base 1316, potentially preventing formation of large thrombi within the recession. Where, in some embodiments, for cross sections taken perpendicular to a direction of elongation of walls 1310 and/or in a proximal-distal direction, recessed portion 1360 is at most 1-30%, or 1-20%, or lower or higher or intermediate percentages or ranges of an area of the wall cross section. Where, in some embodiments, recessed portion 1360 is maximally 0.1-5 mm², 0.1-3 mm² or lower or higher or intermediate ranges or areas in extent for cross sections taken perpendicular to a direction of elongation of walls 1310 and/or in a proximal-distal direction. In some embodiments, recession is to a depth of 0.5-20 mm, or 0.5-10 mm, or 0.5-5 mm, or 1-5 mm, or lower or higher or intermediate ranges or distances.

In some embodiments, an expandable body distal end 1318 includes a top 1380 which partially closes the lumen defined by walls 1310. For example, partially closing leaving a distal opening in expandable device 1308 where a maximal cross sectional dimension 1323 of the opening is 70-99% or 80-99%, or lower or higher or intermediate ranges or percentages, of a maximal cross sectional dimension 1307 of walls 1310.

In some embodiments, device 1302 includes a support 1362 which, in some embodiments, extends from top 1380 and/or walls 1310 at a distal end 1318 of expandable body 1308. In some embodiments, support 1362 is recessed within walls 1110. In some embodiments, moving proximally from distal end 1318, support 1362 extends towards a central region of a volume defined by walls 1310. To form, in some embodiments, a truncated cone portion where the tip of the cone points in a proximal direction. A potential advantage of support 1362 is increase resistance of the device to collapse and/or compaction in a proximal-distal direction. In some embodiments, lack of connection of proximal ends of support 1362 potentially means that there is no connection structure e.g. with associated benefits as described in the next paragraph. This, in some embodiments, at expense of risk of unravelling and/or delamination of wiring at the proximal ends.

A potential advantage of not connecting distal ends of walls 1310 (e.g. to form a top) and/or recessing a distal wall connection within walls 1310 (e.g. as illustrated by connection 1770 FIG. 17B) is reduced risk that the connection protrudes and injures and/or ruptures the aneurysm e.g. as the expandable device is delivered and/or expanded. In some embodiments, expandable body 1308 includes a wall support structure 1362 which, in some embodiments, is a recessed portion extending from top 1380.

Potential advantages of recessed portion/s e.g. portion 1360 and/or portion 1362 include one or more advantage described in the “overview” section of this document. For example, one or more of; positioning the source in a central region of the device, reduced disruption to vessel blood flow of connections to body, increased scaffolding for tissue healing; increased mechanical resistance to collapse of the device.

In some embodiments, expandable device 1302 includes a holder 1306 for a radioactive source (the source is not illustrated in FIGS. 13A-B).

In some embodiments, holder 1306 is attached to base 1316 e.g. to recessed portion central portion 1366 of base 1316. In some embodiments, holder 1306 is held at a central region of expandable device 1308. For example, at a central region of base, for example, where in some embodiments, holder 1336 and/or a holder cross section center 1317 is disposed within a central region of a cross section 1121 of expandable body 1308 where the cross section is taken parallel to base 1316 and/or perpendicular to walls 1310. For example, where holder cross section and/or center 1317 is in a central region of expandable body cross section 1321 e.g. where center 1317 is disposed within a central region of expandable body cross section 1321 width 1307 and/or depth 1360. Where, in some embodiments a central region is defined as being a central 5-80%, or 5-50%, or 5-20%, or lower or higher or intermediate percentages or ranges of a length and/or cross section area.

In some embodiments, holder 1306 includes material which is resistant (e.g. maintains its mechanical characteristics) to ionizing radiation. In some embodiments, holder 1306 includes electrically insulating material (e.g. when electro-chemical detachment is used to detach the push-wire). In an exemplary embodiment, holder 1306 includes polymer (for example, ionizing radiation resistant polymer e.g. PEEK and/or polyimide and/or cyanoacrylate) and, in some embodiments, is formed of polymer. In some embodiments, a radioactive source is mounted on holder 1306, for example, a distal portion of the holder. In some embodiments, holder 1306 connects push-wire 1322 to expandable body 1308. For example, in an exemplary embodiment holder 1306 includes a lumen 1364 into which a distal end of push-wire 1322 is inserted. In some embodiments, connection between push-wire 1322 and holder 1306 is by adhesion. For example, thermal adhesion where the junction is heated to adhere material of holder 1306 onto push-wire 1322. For example, by gluing e.g. where glue is applied to the push-wire and/or support 1306 before contacting them (e.g. inserting push-wire into lumen 1364). In some embodiments, gluing is using cyanoacrylate glue. In some embodiments, at least a portion of holder 1306 is formed from cyanoacrylate where the cyanoacrylate performs both adhesion, mechanical support (including resistance to ionising radiation) and optionally electrolytic and/or electrothermal isolation roles e.g. as described with reference to exemplary holders within this document.

Referring now to FIG. 13C (although feature/s described in this paragraph are also illustrated in FIGS. 13A-B). In some embodiments, holder 1306 is attached to base 1316, for example, by a connector 1366 which, in some embodiments, holds a portion 1368 of base 1316 onto holder 1306. In some embodiments, connector 1366 is ring shaped. Optionally, in some embodiments, connector 1366 includes radiopaque material.

Optionally, in some embodiments, recessed portion 1362 is attached to one or both of base 1366 and/or holder 1306 (not illustrated in FIGS. 13A-B). In some embodiments, portion 1362 is held together with base 1366 onto holder 1306 by connector 1366. Alternatively or additionally, in some embodiments, a second connector (e.g. a second ring) connects portion 1362 to holder 1306.

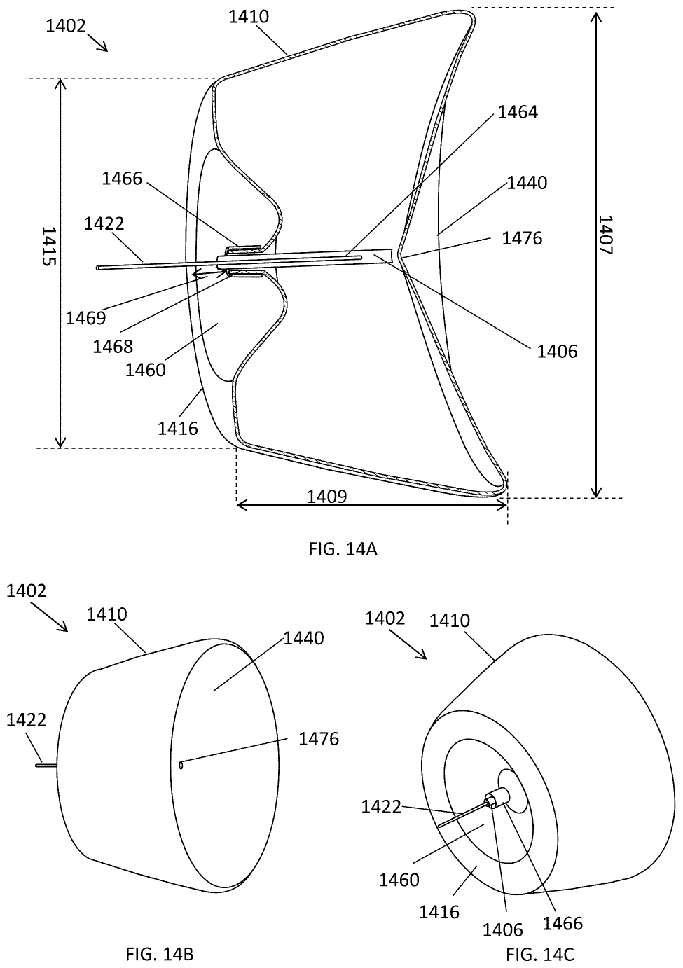

FIG. 14A is a simplified schematic sectional view of an expandable device 1402, according to some embodiments of the invention.

FIGS. 14B-C are simplified schematics of an expandable device 1402, according to some embodiments of the invention.

In some embodiments, FIG. 14A and FIGS. 14B-C illustrate the same expandable device 1402.

In some embodiments, expandable device 1402 includes an expandable body 1408 having a base 1416 (at a proximal end of expandable body 1408), walls 1410 and a top 1440 (at a distal end of expandable body 1408).

In some embodiments, walls 1410 are tubular and have an expanding cross section distally. In some embodiments, a width 1415 of the proximal end of walls 1410 (which, in some embodiments corresponds to a width of base 1416) is larger than a width of a distal end of walls 1407 (which, in some embodiments corresponds to a width of top 1440) where the widths are of the same cross section of the device e.g. as illustrated in FIG. 14A. In some embodiments, walls 1410 gently splay from base 1416 where distal width 1407 is 1.05-2 times, or 1.1-1.5 times, or about 1.3 times, or lower or higher or intermediate multiples or ranges, proximal width 1415.

In some embodiments, a depth 1409 of expandable body 1408 is 0.2-2, or 0.5-1 times or about 0.7 times, or lower, or higher, or intermediate multiples, or ranges of multiples of base width 1407. In some embodiments, a depth 1409 of expandable body 1408 is 0.1-5, or 0.2-1 times or about 0.6 times, or lower, or higher, or intermediate multiples, or ranges of multiples of base width 1407.

In some embodiments, expandable body 1408 is symmetrical in direction/s perpendicular to a proximal-distal direction. For example, a lumen defined by walls 1410 being circular along a length 1409 of the walls. In some embodiments, expandable body 1408 has a truncated cone shape (where the point of the cone is truncated).

In some embodiments, a holder 1406 (including one or more feature as illustrated in and/or described regarding holder 1306 FIGS. 13A-B). Where central positioning of holder 1406 (e.g. as described regarding holder 1306 FIGS. 13A-B) is, in some embodiments, with respect to cross sections of base 1416 and/or top 1440 and/or walls 1410.

In some embodiments, holder 1406 is elongated. In some embodiments, top 1440 includes a top opening 1476. In some embodiments, a source (not illustrated) is attached to holder 1406 by accessing holder 1406 through opening 1476. In some embodiments, a push wire 1422 is connected to holder 1406, at a proximal end of holder 1406.

In some embodiments base 1416 and/or base recessed portion 1460 and/or connection of base 1416 to holder 1406 e.g. by connector 1366 include one or more feature as illustrated in and/or described regarding base 1316 and/or base recessed portion 1360 and/or connection of base 1316 to holder 1306 e.g. by connector 1366 respectively of FIGS. 13A-B.

Optionally, in some embodiments, top 1440 is attached to holder 1306 (not illustrated in FIGS. 13A-B). In some embodiments, top 1440 is held together with base portion 1466 onto holder 1406 by connector 1466. Alternatively or additionally, in some embodiments, a second connector (e.g. a second ring) connects top 1440 to holder 1406.

Referring now to FIG. 14C, in some embodiments, holder 1406 protrudes from recessed base portion 1460.

In some embodiments, recession of connector 1468 (or any of elements 1204, 1368, 1568, 1766) is to a depth 1469 which is, in some embodiments, 0.1-5 mm, or 0.1-2 mm, or 0.1-1 mm, or lower or higher or intermediate depths or ranges. Where the term connector is, at least, in this paragraph and the paragraph below this paragraph, in some embodiments, used to refer to element/s of the device which connect material (e.g. mesh material of the device) together and/or to element/s which are rigid e.g. in comparison to material of the body of the device (e.g. mesh).

In some embodiments, material of the device is recessed further than a depth of the connector 1468 and then curves to meet the connector 1468, the maximal depth of recession of the material being e.g. from a surface of a base of the device being, in some embodiments, 0.1-10 mm, or 0.5-5 mm, or 1-5 mm, or lower or higher or intermediate distances or ranges.

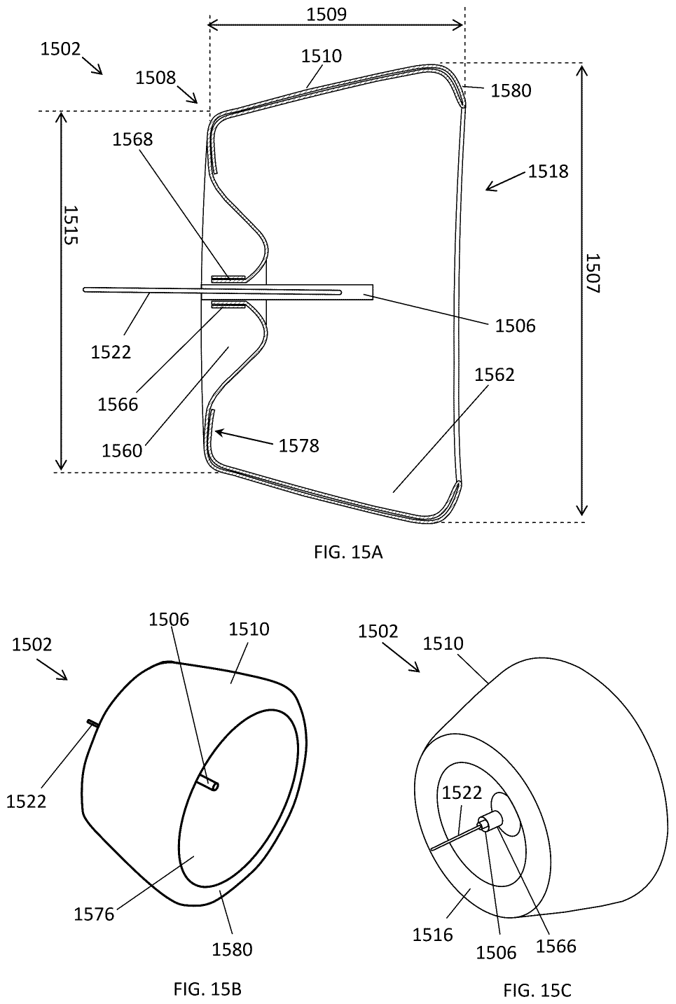

FIG. 15A is a simplified schematic sectional view of an expandable device 1502, according to some embodiments of the invention.

FIGS. 15B-C are simplified schematics of an expandable device 1502, according to some embodiments of the invention.

In some embodiments, FIG. 15A and FIGS. 15B-C illustrate the same expandable device 1502.

In an exemplary embodiment, an expandable device 1502 includes walls 1510, a base 1516, a partially open distal end 1518, and a centrally positioned holder 1506.

Where, in some embodiments, expandable body 1508 walls 1510 have a shape including one or more feature as illustrated for and/or described regarding walls 1410 FIGS. 14A-C. For example, expandable body 1508, in some embodiments, has a truncated cone shape.

Where, in some embodiments, base 1516 includes one or more feature as illustrated in and/or described regarding base 1316 of expandable device 1302 FIGS. 13A-B. For example where recessed portion 1560 and/or connecting portion 1566 correspond to recessed portion 1360 and/or portion 1366.

Where, in some embodiments, partially open distal end 1518 of expandable device 1502 includes one or more feature as illustrated in and/or described regarding partially open distal end 1318 of expandable device 1302 FIGS. 13A-B.

Where, in some embodiments, holder 1506 includes one or more feature illustrated in and/or described regarding holder 1306 FIGS. 13A-C and/or regarding position of holder 1406 FIG. 14A (e.g. with respect to expandable body 1408 portion/s).

In some embodiments, expandable device 1502 includes a support 1562 which extends proximally from (and is optionally attached to) top 1540, support 1562 extending towards base 1516, and closely following (and/or in contact with) walls 1510. In some embodiments, support 1562 extends to and closely follows (and or is in contact with) a portion of base 1516, providing a double layered base region 1578. In some embodiments, support 1562 closely follows (e.g. is at most 0.5 mm, or 0.3 mm or 0.2 mm, or 0.01-0.5 mm, or 0.01-0.2 mm, or 0.05-0.2 mm, or 0.1-0.2 mm, or lower or higher or intermediate distances or ranges away from walls 1510 and/or base 1516) and/or is in contact with surfaces of walls 1510 and/or base 1578.

In some embodiments, support 1562 is recessed within walls 1510 (e.g. as illustrated in FIG. 15A). Within walls 1510 supports walls 1510 and/or base 1516. Alternatively, in some embodiments, support 1562 is disposed externally to walls 1510 and/or is proximal of base 1516.

Optionally, in some embodiments, support 1562 extends (e.g. closely following base 1516 e.g. including base recessed portion 1560) to holder 1506.

In some embodiments, support 1562 ends 1578 at an external region of base 1516. A potential advantage being reduced screening of radioactive emissions from the source connected to holder 1516 by the double layering provided by support 1562. In some embodiments, support 1562 ending before a central region of base 1516 is as mechanical support and/or reduced porosity is provided by denser wire distribution of base 1516 at central regions of the device e.g. associated with properties of expanding mesh structures.

Optionally, in some embodiments, support 1562 extends centrally e.g. along base recessed portion 1560 and is connected to holder 1506.

FIG. 16A is a simplified schematic sectional view of an expandable device 1602, according to some embodiments of the invention.

FIG. 16B is a simplified schematic view of an expandable device 1602, according to some embodiments of the invention.

In some embodiments, FIG. 16A and FIG. 16B illustrate the same expandable device 1602.

In an exemplary embodiment, an expandable device 1602 includes walls 1610, a base 1616, a centrally positioned holder 1606, a partially closed top 1640 and a support 1662.

Where, in some embodiments, walls 1610 have a shape including one or more feature as illustrated for and/or described regarding walls 1310 FIGS. 13A-B.

Where, in some embodiments, base 1616 including one or more feature as illustrated in and/or described regarding base 1316 FIGS. 13A-B.

Where, in some embodiments, support 1662 extends from top 1640 and/or follows walls 1610 as illustrated in and/or described regarding support 1562 and walls 1510 FIG. 15A.

In some embodiments, a source 1604 is attached to holder 1606. Where one or more feature of the attachment and/or source is as illustrations referred to in and/or described in the sections of this document entitled “ ”. In an exemplary embodiment, source 1604 is a rhodium strip which is irradiated with Pd-103. In some embodiments, source 1604 is attached to holder 1606 by winding the strip onto the holder. Alternatively or additionally, source 1604 is provided as a coil and is then fitted onto holder 1606 e.g. with an interference fit associated with sizing of the holder 1606 and/or source 1604.

In some embodiments, support walls 1662 do not extend to base 1616. In some embodiments, support 1662 does not extend to a proximal portion of walls 1610. A potential advantage being reduced screening of radioactive emissions from source 1604 by support 1662 in a direction towards aneurysm opening.

Optionally, in some embodiments, expandable device 1602 includes one or more distal support 1682. In some embodiments, distal support 1682 is configured to hold expanding device 1602 in position within an aneurysm, for example holding device 1602 in position blocking an opening to the aneurysm. In some embodiments, distal support 1682 is an expandable structure which expands distally, for example, to contact and/or apply pressure to a top of the aneurysm. In some embodiments, an induced reactive force to the pressure, from the aneurysm on the device pushes the device into close contact with the aneurysm opening. In an exemplary embodiment, distal support 1682 is a spring. In some embodiments, distal support 1682 is connected to holder 1606. Alternatively or additionally, in some embodiments, distal support is attached to one or more of base 1616, walls 1610 and top 1640.

In some embodiments, in a collapsed configuration (e.g. of distal support 1682 and/or of expandable device 1602) distal support 1682 is recessed within walls 1610.

In some embodiments, the expandable devices illustrated in FIGS. 1, 4A-H, 5-10, 11, 12A-F, 13A-C, 14A-C, 15A-C, 17A-B, 18 include a distal support e.g. including feature/s as illustrated in and/or described regarding distal support 1682 FIG. 16A-B.

FIG. 17A is a simplified schematic of an expandable device 1702, according to some embodiments of the invention.

FIG. 17B is a simplified schematic sectional view of an expandable device 1702, according to some embodiments of the invention.

In some embodiments, FIG. 17A and FIG. 17B illustrate the same expandable device 1702.

Device 1702, in some embodiments, is described with respect to description in other portion/s of this document as having walls 1710 and a base 1716 to which a holder is attached 1706 and a proximally positioned support structure including support walls 1786 and a support base 1784. Where a push wire 1722 is attached to the support structure 1738.

Alternatively, in some embodiments, device 1702 is described with respect to description in other portion/s of this document as having a base 1784, a recessed portion 1788 of base 1784 to which push wire 1722 is attached. Where base 1784 is connected to walls 1786 which are closed by a top 1710 to which holder 1706 is connected.

Where, for example, recessed base 1788 includes one or more feature as illustrated in and/or described regarding 1360 FIGS. 14A-B.

Where, for example, a shape of walls 1786 includes one or more feature as illustrated in and/or described regarding walls 1410 FIGS. 14A-C.

Where, for example, recessed top 1710 includes one or more feature as illustrated in and/or described regarding 1362 FIGS. 13A-B.

FIG. 17B illustrates an embodiment, where holder 1706 is not directly connected to push-wire 1722 and/or proximal portion/s 1784, 1788 potentially increasing flexibility of device 1702 e.g. during delivery through tortuous vasculature and/or increasing ability of device 1702 to conform to a shape of an aneurysm.

In some embodiments, holder 1706 and push wire connector 1774 are a connected single piece (e.g. including one or more feature as illustrated in and/or described regarding holder 1306 FIGS. 13A-B and/or holder 1406 FIGS. 14A-C and/or holder 1506 15A-C and/or holder 1606 16A-B).

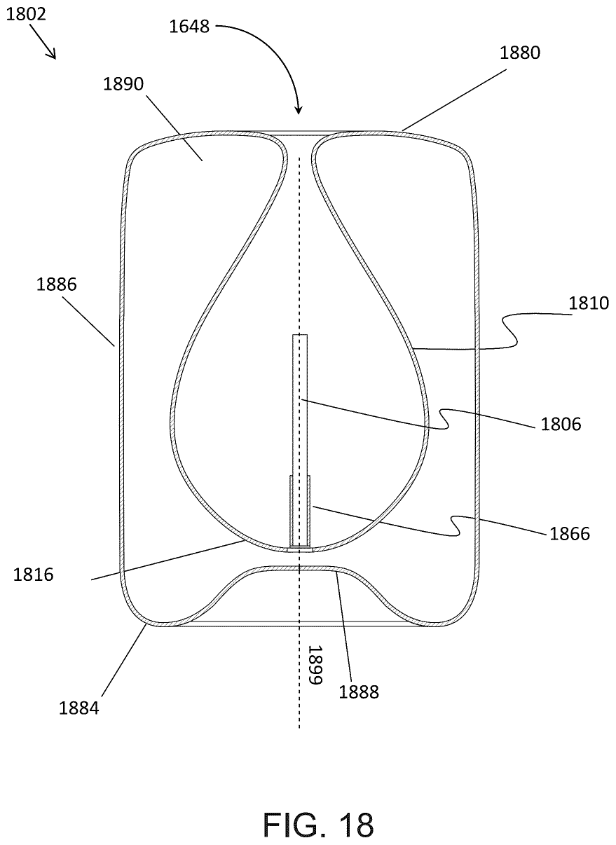

FIG. 18 is a simplified schematic cross sectional view of an expandable device 1802, according to some embodiments of the invention.

In some embodiments, expandable device 1802 includes walls 1810, a base 1816 and a support including support walls 1886 and a support base 1184 where the support extends around walls 1810 and base 1816. The support, for example, including one or more features as described and/or illustrated regarding support 1738 FIG. 17B.

In some embodiments, support walls 1886 are tubular in shape e.g. cylindrical and/or where the expandable body 1808 has rotational symmetry about central longitudinal axis 1899. In some embodiments, a shape of walls 1886 includes one or more feature as described and/or illustrated regarding walls 1310 FIG. 13A-B.

In some embodiments, a push-wire (not illustrated) is connected to a holder 1806. Extending, in some embodiments, through opening 1648. Alternatively, in some embodiments, the push wire extends from holder 1806 in an opposite direction, connected to and/or passing through a recessed base support portion 1888.

In some embodiments, holder 1806 is attached to base 1816 by connector 1866. Where, in some embodiments, connector 1866 is attached to base 1816 by one or more of crimping, welding and quenching and in an exemplary embodiments, is attached by quenching.

In some embodiments, holder 1806 hosts one or more radioactive source (not illustrated). In some embodiments, holder 1806 includes PEEK and/or cyanoacrylate and/or includes one or more feature as illustrated and/or described regarding holder/s elsewhere in this document.

### Exemplary Materials

### Exemplary Expandable Body Materials

In some embodiments, an expandable body of an expandable device is formed from mesh e.g. a mesh constructed of wires.

In some embodiments, porosity of one or more portion of the expandable device body, in an expanded conjuration (e.g. relaxed expanded, and/or expanded within an aneurysm) is 5-90%, or 10-90%, or 10-80%, or 20-80%, or 30-80%, or 20-70%, or lower or higher or intermediate ranges or percentages.

In some embodiments, pore density of one or more portion of the expandable device body, in an expanded conjuration (e.g. relaxed expanded, and/or expanded within an aneurysm) is 1-500, or 5-400 pores/mm².

In some embodiments, at least a portion of the wires are formed from nitinol. In some embodiments, at least a portion of the wires are nitinol with an additional material (also herein termed “composite wires”) where exemplary additional materials include platinum and tantalum.

For example, in some embodiments, wires include nitinol and a core of a different material (e.g. platinum and/or tantalum). In some embodiments, wires including nitinol and an additional material are 50-80% nitinol and 20-50% the additional material (by weight and/or volume). For example, in some embodiments, wires of the mesh include DFT® wire (Drawn Filled Tubing) from Fort Wayne Metals Research Products Corp.

In some embodiments, a mesh includes both nitinol wires and composite wires (e.g. DFT® wires). In an exemplary embodiment, at least a portion of an expandable body (e.g. the entire expandable body) is formed from 50-80% nitinol wires and 20-50% composite wires. For example, in a 96 wire device, in some embodiments, 64 of the wires are nitinol wires and 32 of the wires are composite wires.

In some embodiments, a single layer of mesh forming at least a portion of an expandable device includes 72-288 wires in the layer. In some embodiments, a multi-layer mesh (e.g. double layer mesh) forming at least a portion of an expandable device has 64-96 wires in each layer.

In an exemplary embodiment, mesh is of a ½ type woven mesh. Where, in some embodiments, wires in one direction pass under two wires and then over two wires in the other direction. Other types of mesh, for example, 1/1, are envisioned and encompassed.

In some embodiments, devices including more than one layer (e.g. FIG. 16A-B layers 1610, 1662) close mesh layers act to increase wire numbers of the mesh. For example, in some embodiments, a single layer mesh has 64-96 wires. In some embodiments, close stacking of mesh layers acts to double mesh density e.g. for 64-96 wire layers, to an effective 128-192 wire layer.

### Exemplary Connector Materials

In some embodiments, connector/s e.g. one or more of connector 1266 FIGS. 12C-F, connector 1366 FIGS. 13A-C, connector 1466 FIGS. 14A and 14C, connector 1566 FIGS. 15A and 15C, connector 1666 FIG. 16A, connector 1766 and FIG. 17B connector 1770 FIG. 17B include radiopaque material. For example, include a platinum/iridium alloy and/or Tantalum. In some embodiments, the connector/s include non-radiopaque material, for example, one or more of stainless steel, nitinol, titanium, cobalt-chrome (CoCr) alloy, polymer (e.g. PEEK and/or polyimide).

### Exemplary Sources and Exemplary Source Attachment

In many embodiments, a source is attached to a holder. In some embodiments, one or more property of holders in this section includes feature/s as described and/or illustrated regarding exemplary holders referred to within this document e.g. holder 1106 FIG. 11 and/or holder 1206 FIGS. 12A-H and/or holder 1606 FIGS. 16A-B.

FIG. 19A is a simplified schematic of a source holder 1906, according to some embodiments of the invention.

FIG. 19B is a simplified schematic of a radioactive source 1904, according to some embodiments of the invention.

FIG. 19C is a simplified schematic of a radioactive source 1904 connected to a source holder 1906, according to some embodiments of the invention.

In some embodiments, FIG. 19C illustrates source holder 1906 of FIG. 19A and source 1904 of FIG. 19B.

Referring now to FIG. 19B which illustrates an exemplary source 1904. In some embodiments, source 1904 (e.g. for device/s described in this document) has a body including a first material, which has been treated with radioactive material.

In some embodiments, a shape of source body 1904 is elongate (e.g. as illustrated in FIG. 19A-C) in one or more direction. In some embodiments, source body/ies 1904 are spherical, or rod-shaped, or are coils (e.g. as described later within this section).

In some embodiments, the body is treated with one or more radioisotope (e.g. to become a source) e.g. Pd-103 and/or I-125. Where treating, for example, includes coating the body in a material containing the radioisotope and/or performing ion implantation, and/or radioactive activation via proton and/or neutron bombardment.

In an exemplary embodiment, the body includes (e.g. is formed from) rhodium. Where, in some embodiments, the rhodium has been treated (e.g. by proton bombardment e.g. in a cyclotron) to include radioactive Pd-103 isotopes.

In some embodiments, the source includes an encapsulated seed.

In some embodiments, the source includes a commercially available brachytherapy seed e.g. ISOAID Advantage™ IAI-125A, ISOAID Advantage™ IAPd-103A.

In some embodiments, the source includes a seed (optionally encapsulated) which is 0.05-2 mm, or 0.1 mm-1 mm, or lower or higher or intermediate dimensions or ranges, in at least one dimension, e.g. 0.05-2 mm, 0.1-1 mm in two dimensions or lower or higher or intermediate dimensions or ranges e.g. 0.05-2 mm, 0.1-1 mm or lower or higher or intermediate dimensions or ranges in all three dimensions.

In some embodiments, emissions from source for an expandable device are selected based on a size of an aneurysm to be treated, a shielding of the expandable device when the device is within the aneurysm to be treated and a position of the source with respect to the device and/or with respect to the aneurysm, when the device is in position.

For example, referring devices with width of 5 mm, 7 mm, 9 mm, 11 mm and 14 mm.

In some embodiments, a source length for exemplary devices, is selected based on device dimension/s. Where, in some embodiments, one or more source characteristic remains the same (e.g. cross sectional dimension, radiation per volume and/or surface area of source). Where, for example, in some embodiments, a kit including a variety of sizes of device has sources scaled (e.g. by length) to be proportional to one or more dimension of the device e.g. in a relaxed expanded configuration and/or in expanded configurations with aneurysms.

For example, in some embodiments, one or more dimension of a device e.g. device expanded width and/or length and/or depth is 1-20, or 1-10, or 2-10, or lower or higher or intermediate times a central longitudinal axis length of the source.

For example, in some embodiments, a 5 mm width and/or length device has a source of source length 0.5-2 mm. For example, in some embodiments, a 7 mm width and/or length device has a source length of 1-3 mm. For example, in some embodiments, a 9 mm width and/or length device has a source length of 1.5-4 mm. For example, in some embodiments, an 11 mm width and/or length device has a source length of 3-7 mm. For example, in some embodiments, a 14 mm width and/or length device has a source length of 3.5-10 mm.

In some embodiments, source holder 1906 includes a lumen 1964 configured to receive one or more source. In some embodiments, holder 1906 includes arms 1936 (e.g. including one or more feature illustrated and/or described regarding arms 1136 FIG. 11). In some embodiments, source holder 1906 includes openings 1937 in the material of the holder potentially allowing escape of radiation un-attenuated by holder material from these openings.

In some embodiments, holder 1906 includes radiation blocking material, for one or more region of the holder. Potentially enabling control of directionality of radiation. For example, in some embodiments, radiation is screened in a direction towards optical nerve/s.

Referring now to FIG. 19C in some embodiments, source 1904 remains within holder 1906 at one or more end of holder by size and/or shape matching between the holder lumen 1964 and source 1904 e.g. holder walls are stretched during insertion of source 1904 into lumen 1964. Alternatively or additionally, holder is closed at one or both end e.g. by cap/s and/or deformed to reduce lumen 1964 size potentially preventing exit of the source from the holder.

In some embodiments, source 1904 has a shorter length than holder lumen 1964 (e.g. as illustrated in FIGS. 19A-B). In some embodiments, a position of source 1904 along a length of holder 1906 is selected e.g. before insertion of the device into a subject.

FIG. 20A is a simplified schematic of a source holder 2006, according to some embodiments of the invention.

FIG. 20B is a simplified schematic of a radioactive source 2004, according to some embodiments of the invention.

FIG. 20C is a simplified schematic of a radioactive source 2004 connected to a source holder 2006, according to some embodiments of the invention.

In some embodiments, source 2004 is attached to holder 2006 externally. In an exemplary embodiment, source 2004 is an elongated element which is disposed by being wrapped around holder 2006. In some embodiments, source 2004 is a coil e.g. wire as illustrated in FIG. 20B which is pushed onto support 2006. In some embodiments, inner dimension/s of source 2004 (and/or source 2104) are smaller than outer dimension/s of support 2006 and source is deformed (elastically and/or plastically) to fit onto support 2006. Reactive force, in some embodiments, acting to hold source 2004 (and/or source 2104) in position on support 2006.

In some embodiments, more than one source is attached to support 2006 e.g. in this way. For example, including one or more feature as illustrated in and/or described regarding FIGS. 29A-B.

FIG. 21 is a simplified schematic of a source 2104, according to some embodiments of the invention. In some embodiments, source is a tape coil 2104. In some embodiments, source 2104 is connected to a support e.g. including one or more feature illustrated and/or described regarding attachment of source 2024 to support 2006 FIGS. 20A-C. In some embodiments, tape coil 2104 and/or wire coil 2004 sources are attached to a holder by being inserted into a lumen of the holder and, in some embodiments, held within the lumen by a cap and/or distortion of the lumen.

FIG. 22A is a simplified schematic of a radioactive source 2204, a source holder 2406 and a source holder cap 2234, according to some embodiments of the invention.

FIG. 22B is a simplified schematic of a radioactive source 2204 connected to a source holder 2206, according to some embodiments of the invention.

In some embodiments, source 2204 is held within a holder lumen 2264 by cap 2234. Where, in an exemplary embodiments, cap 2234 fixes to holder 2206 by screwing where cap 2234 and/or holder 2206 includes threading 2297. Alternatively, or additionally, in some embodiments, cap 2234 is glued into position. Alternatively or additionally in some embodiments, cap 2234 and/or sized and/or shaped to have a portion insertable into lumen 2264 but difficult to remove e.g. having an interference fit with holder 2206. In some embodiments, cap 2234 is closed and/or closes lumen 2264. Alternatively, in some embodiments, cap includes one or more opening sized to be too small to allow passage of source 2204.

Optionally, in some embodiments, element 2204 as illustrated in FIGS. 22A-B is a second holder, where a source is attached externally to element 2204, where both element 2204 and a source (e.g. wrapped around, e.g. a coil source) coupled to element 2204 are held within lumen 2264.

FIGS. 23-26, in some embodiments, illustrate exemplary positions for a radioactive source with respect to a holder and/or connector.

In some embodiments, like elements of FIGS. 23-26 are indicated with like reference numerals, e.g. element 2304 corresponding to element 2504. Where “like” is herein defined as sharing one or more feature as illustrated in the figure/s and/or described regarding the figures in the text of this application.

FIG. 23 is a simplified schematic of a portion of an expandable device, according to some embodiments of the invention.

In some embodiments, a portion of an expandable body 2308 of an expandable device is held (e.g. closing a base or top) by a connector 2366. Where, in some embodiments, connector 2366 has a ring shape. In some embodiments, connector 2366 is attached by one or more of crimping, welding, quenching. In some embodiments, a holder 2306 is attached to expandable body 2308 by connector 2366, for example where (e.g. as illustrated in FIG. 23) expandable body 2308 is disposed between holder 2306 and connector 2366. Optionally, in some embodiments, connector includes radiopaque material.

Optionally, in some embodiments, holder 2306 provides connection of the expandable device to a push-wire 2322. In some embodiments, connection is via a detachment region 2392. Where, in some embodiments, detachment region 2392 is configured to be one or more of electrolytically and electrothermally weakened to release a proximal portion of push-wire 2322 from the expandable device. In some embodiments, detachment region 2392 includes a mechanical attachment which, in some embodiments is released for removal of push-wire 2322. Where, in some embodiments, mechanical attachment includes a screw attachment where push-wire is rotated to detach. In some embodiments, mechanical detachment includes a pull and/or push wire activated release, where the device includes an additional control wire e.g. extending through the catheter for control by a user.

In some embodiments, holder 2306 provides electrical insulation between detachment region 2392 and/or push-wire 2322 and expandable body 2308 (and/or other regions of the device). A potential benefit being the ability to detach the push-wire using electrical stimulation, without providing the stimulation to the rest of the device.

In some embodiments, a source 2304 is attached to holder 2306 e.g. externally e.g. including one or more feature as described regarding sources 2004, 2104 and holder 2006 FIGS. 20A-C and FIG. 21. Alternatively or additionally, a source is attached to holder 2306 by being inserted at least partially into a lumen of the holder (e.g. as described and/or illustrated regarding holder 1106 FIG. 11 and/or holder 1906 and/or source 1904 FIGS. 19A-C).

In some embodiments, source 2304 is attached to a portion of holder 2306 distal of connection of the holder to expandable body 2308 e.g. potentially positioning source 2304 in a central region of the device e.g. as described elsewhere in this document.

In some embodiments, connector 2366 includes radiopaque material meaning that potentially, it blocks radiation emitted by source 2304 distally. In some embodiments, the source is positioned proximally and/or externally to connector 2366.

FIG. 24 is a simplified schematic of a portion of an expandable device, according to some embodiments of the invention.

In some embodiments, FIG. 24 illustrates an embodiment where a source 2404 is attached externally to a holder 2406, between holder 2406 and a connector 2466 and/or mesh of an expandable body 2408. Where connector 2466 includes radiopaque and/or radiation attenuating material, potentially position of source 2404 within connector 2466 reduces radiation emanated (and received by tissue) in a direction perpendicular to the proximal-distal direction (where push-wire 2422 is disposed at a proximal end of the illustration of FIG. 24 and extends in a distal direction) and/or in an axial direction (with respect to a ring shaped connector 2466).

FIG. 25 is a simplified schematic of a portion of an expandable device, according to some embodiments of the invention.

In some embodiments a source 2504 is attached externally to a connector 2466. Potentially reducing attenuation of radiation emitted by source 2504 by connector e.g. as opposed to the configuration illustrated in FIG. 24.

FIG. 26 is a simplified schematic of a portion of an expandable device, according to some embodiments of the invention.

In some embodiments, a source 2604 is attached proximally of a connector 2666. If connector 2466 includes radiation attenuating material, this proximal attachment of source means that radiation directed proximally e.g. towards a neck of an aneurysm is not attenuated by connector 2666.

Exemplary Multiple Sources and/or Multiple Source Devices

FIG. 27 is a simplified schematic cross sectional view of an expandable device 2702, according to some embodiments of the invention.

In some embodiments, expandable device 2702 includes more than one source 2704a, 2704b. In some embodiments, one or more source 2704a is selected and/or positioned for irradiation of an aneurysm sac and/or internal volume. In some embodiments, one or more source 2704b is selected for irradiation of an aneurysm neck region. In some embodiments, more than one source 2704a, 2704b is hosted by a single holder 2706.

FIG. 28 is a simplified schematic cross sectional view of an expandable device 2802, according to some embodiments of the invention.

In some embodiments, expandable device 2802 includes a plurality of sources 2804a-d. In some embodiments, one or more expandable element 2863 is configured to position a source 2804c attached to expandable element 2863 e.g. within a volume defined by expandable body 2808. In some embodiments, each source 2804a-d is connected to holder 2806 by an individual expandable element (e.g. as illustrated in FIG. 28).

### Exemplary Shielding

For example, as described elsewhere in this document e.g. with respect to 2246 connector of FIGS. 22A-B being radiation blocking. In some embodiments, a source is shielded from irradiating in one or more direction. Where, in some embodiments, shielding includes attenuating the emitted radiation to generate a reduced dose and/or dose rate in one or more direction. Where, in some embodiments, shielding includes blocking emitted radiation.

In some embodiments, radiopaque materials are used for shielding e.g. connector 2246 including platinum.

In some embodiments, doped PMMA is used for shielding. For example, including one or more feature as described and/or illustrated in “Nuclear Engineering and Technology, Volume 52, Issue 11, November 2020, Pages 2613-2619 “Gamma radiation shielding properties of poly (methyl methacrylate)/Bi203 composites” by Da Cao, Ge Yang, Mohamed Bourham, Dan Moneghan” which is herein incorporated by reference in its entirety.

### Exemplary Radiation Levels

In some embodiments, total dose is measured using one or more technique as known in the art of radioactive source measurement. For example, total dose being measured from source, by using one or more of source isotope half-life time, known activity, and measurement time e.g. using a TG-43 algorithm as known in the art of dose calculation for radioactive sources.

In some embodiments, a neck of the aneurysm receives a desired dose e.g. of 10-40 Gy (and/or doses as described elsewhere in this document).

In some embodiments, the desired dose received is related to one or more of a plurality of device characteristics. For example, where, for a given source, the dose received by the neck of the aneurysm is related to a distance of the source from the neck and strength of any shielding between the source and the neck. Where, shielding in some embodiments, is provided by material of the expandable body and/or one or more additional element (e.g. marker, connector).

Where, in some embodiments, possible distances of the neck from the source are defined by a size of the aneurysm and/or maximal radiation dose to be tolerated by tissue outside the aneurysm. Where the radiation dose received by tissue outside the aneurysm is also affected by any shielding between the source and the tissue (e.g. as provided by a body of the expandable device therebetween). In some embodiments, the neck is 1-10 mm from the source.

In some embodiments, radioactive source/s for devices and/or exemplary radiation doses and/or dose rate/s for treatment (e.g. of aneurysm) as described within this document include one or more feature as described and/or illustrated regarding seed/s and/or sources within US Patent Publication No. US2020/0078602 which is herein incorporated by reference in its entirety.

In some embodiments, one or more portion of the expandable body absorbs 10-90%, or 20-80%, or 30-70%, or lower or higher or intermediate percentages or ranges. In some embodiments, one or more portion of the expandable body absorbs 20-80% of emitted radiation from a source incident on the expandable body, or lower or higher or intermediate percentages of radiation emitted from the source. For exemplary doses listed below, if, in some embodiments, the mesh is disposed at less that 5 mm away from an outer surface of the source, the dose levels listed are reduced by 20-80% at 5 mm and 10 mm the reduction, in some embodiments, reflecting absorption by the expandable body of the device. If the mesh is disposed between 5 mm and 10 mm then the dose levels listed at 10 mm, in some embodiments, are reduced by 20-80%.

In some embodiments, a radiation source provides, at 0-1 mm from an outer surface of the source, a dose of 100-500 Gy or lower or higher or intermediate ranges or doses. In some embodiments, a radiation source provides a dose of 5-130 Gy, at 5 mm from an outer surface of the source or lower or higher or intermediate ranges or doses. In some embodiments, a radiation source provides a dose of 1-20 Gy, or lower or higher or intermediate ranges or doses, at 10 mm from an outer surface of the source.

In some embodiments, a radiation source provides, at 0-1 mm from an outer surface of the source, a dose of 100-500 Gy or lower or higher or intermediate ranges or doses, and a dose of 5-130 Gy, at 5 mm from an outer surface of the source or lower or higher or intermediate ranges or doses, and a dose of 1-20 Gy, or lower or higher or intermediate ranges or doses, at 10 mm from an outer surface of the source.

In some embodiments, a radiation source provides, at 0-1 mm from an outer surface of the source, a dose of 100-500 Gy or lower or higher or intermediate ranges or doses, a dose of 15-75 Gy, at 5 mm from an outer surface of the source or lower or higher or intermediate ranges or doses, and a dose of 2-10 Gy, or lower or higher or intermediate ranges or doses, at 10 mm from an outer surface of the source.

In some embodiments, a radiation source provides, at 0-1 mm from an outer surface of the source, a dose of 100 Gy, at 5 mm from an outer surface of the source, a dose of 15 Gy, and a dose of 2 Gy at 10 mm from an outer surface of the source.

In some embodiments, a radiation source provides, at 0-1 mm from an outer surface of the source, a dose of 200 Gy, at 5 mm from an outer surface of the source, a dose of 30 Gy, and a dose of 4 Gy at 10 mm from an outer surface of the source.

In some embodiments, a radiation source provides, at 0-1 mm from an outer surface of the source, a dose of 300 Gy, at 5 mm from an outer surface of the source, a dose of 45 Gy, and a dose of 6 Gy at 10 mm from an outer surface of the source.

In some embodiments, a radiation source provides, at 0-1 mm from an outer surface of the source, a dose of 400 Gy, at 5 mm from an outer surface of the source, a dose of 60 Gy, and a dose of 8 Gy at 10 mm from an outer surface of the source.

In some embodiments, a radiation source provides, at 0-1 mm from an outer surface of the source, a dose of 500 Gy, at 5 mm from an outer surface of the source, a dose of 75 Gy, and a dose of 10 Gy at 10 mm from an outer surface of the source.

In some embodiments, a radiation source provides, at 0-1 mm from an outer surface of the source, a dose of 100-500 Gy or lower or higher or intermediate ranges or doses, a dose of 30-150 Gy, at 5 mm from an outer surface of the source or lower or higher or intermediate ranges or doses, and a dose of 4-20 Gy, or lower or higher or intermediate ranges or doses, at 10 mm from an outer surface of the source.

In some embodiments, a radiation source provides, at 0-1 mm from an outer surface of the source, a dose of 100 Gy, at 5 mm from an outer surface of the source, a dose of 30 Gy, and a dose of 4 Gy at 10 mm from an outer surface of the source.

In some embodiments, a radiation source provides, at 0-1 mm from an outer surface of the source, a dose of 200 Gy, at 5 mm from an outer surface of the source, a dose of 60 Gy, and a dose of 8 Gy at 10 mm from an outer surface of the source.

In some embodiments, a radiation source provides, at 0-1 mm from an outer surface of the source, a dose of 300 Gy, at 5 mm from an outer surface of the source, a dose of 90 Gy, and a dose of 12 Gy at 10 mm from an outer surface of the source.

In some embodiments, a radiation source provides, at 0-1 mm from an outer surface of the source, a dose of 400 Gy, at 5 mm from an outer surface of the source, a dose of 120 Gy, and a dose of 16 Gy at 10 mm from an outer surface of the source.

In some embodiments, a radiation source provides, at 0-1 mm from an outer surface of the source, a dose of 500 Gy, at 5 mm from an outer surface of the source, a dose of 150 Gy, and a dose of 20 Gy at 10 mm from an outer surface of the source.

In some embodiments, a radiation source provides, at 0-1 mm from an outer surface of the source, a dose of 100-500 Gy or lower or higher or intermediate ranges or doses, a dose of 5-25 Gy, at 5 mm from an outer surface of the source or lower or higher or intermediate ranges or doses, and a dose of 2-5 Gy, or lower or higher or intermediate ranges or doses, at 10 mm from an outer surface of the source.

In some embodiments, a radiation source provides, at 0-1 mm from an outer surface of the source, a dose of 100 Gy, at 5 mm from an outer surface of the source, a dose of 5 Gy, and a dose of 1 Gy at 10 mm from an outer surface of the source.

In some embodiments, a radiation source provides, at 0-1 mm from an outer surface of the source, a dose of 200 Gy, at 5 mm from an outer surface of the source, a dose of 10 Gy, and a dose of 2 Gy at 10 mm from an outer surface of the source.

In some embodiments, a radiation source provides, at 0-1 mm from an outer surface of the source, a dose of 300 Gy, at 5 mm from an outer surface of the source, a dose of 15 Gy, and a dose of 3 Gy at 10 mm from an outer surface of the source.

In some embodiments, a radiation source provides, at 0-1 mm from an outer surface of the source, a dose of 400 Gy, at 5 mm from an outer surface of the source, a dose of 20 Gy, and a dose of 4 Gy at 10 mm from an outer surface of the source.

In some embodiments, a radiation source provides, at 0-1 mm from an outer surface of the source, a dose of 500 Gy, at 5 mm from an outer surface of the source, a dose of 25 Gy, and a dose of 5 Gy at 10 mm from an outer surface of the source.

In some embodiments, radiation source/s of an expandable device (e.g. expandable devices as described within this document) are configured to irradiate a region of the aneurysm sac and/or neck, while minimally radiating tissue outside the aneurysm. For example, where, the aneurysm has an average (and/or maximum) dimension of 5 mm, in some embodiments, a radioactive dose at 5 mm from a source central axis is 15% of the dose at 1 mm from the source central axis. In some embodiments, a radioactive dose at 10 mm away from a source central axis, and/or a source outer surface is 0.5% of a dose 1 mm from the source.

In some embodiments, a radioactive source is 0.1-10 mm, or 1-5 mm long, or lower or higher or intermediate ranges or lengths. In some embodiments, the source is elongate. In some embodiments, a width (e.g. maximal and/or average cross sectional dimension e.g. taken perpendicular to a length of the source) is 0.1-1 mm, or 0.15-0.5 mm wide or higher or lower or intermediate ranges or widths. In some embodiments, a radioactive source is 0.1-10 mm long and 0.1-1 mm wide. In some embodiments, a radioactive source is 1-5 mm long and 0.15-0.5 mm wide.

In some embodiments, the radioactive source includes Iodine-125 (I-125). For example, a titanium encapsulated I-125 seed. Where, in some embodiments, the seed is 0.5-1 mm in average and/or maximum cross sectional dimension (e.g. diameter) and/or 5-10 mm in length. In some embodiments, the radioactive source is a commercially available Iodine-125 brachytherapy seed, produced for use in brain brachytherapy.

In some embodiments, a radioactive dose at 5 mm from a source central axis is 15% of the value at 1 mm, and the dose at 10 mm is 4% of the dose at 1 mm.

In some embodiments, a radioactive source includes Palladium-103. Where, in some embodiments, a radioactive dose at 10 mm away from a source outer surface is 0.5% of a dose 1 mm from the source.

In some embodiments, energies of the gamma isotopes are 20-30 KeV. In some embodiments 4-15, or 10-15 mm, or lower or higher or intermediate distances or ranges away from the source all of the radiation is absorbed. In some embodiments, a radioactive dose received by tissue at 4-15, or 10-15 mm, or lower or higher or intermediate distances or ranges away from an outer surface of the source is less than 1% of the total dose received by tissue.

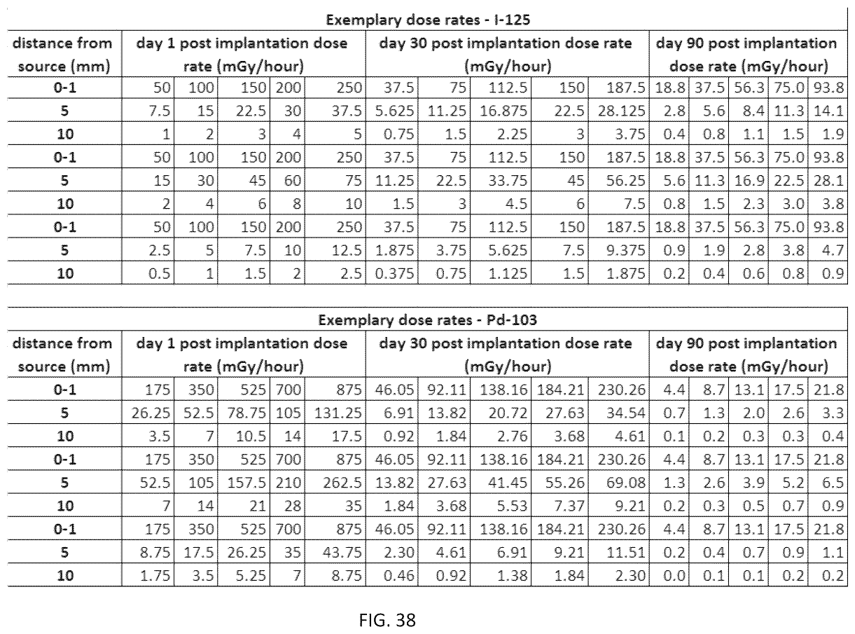

FIG. 38 shows tables illustrating exemplary dose rates, according to some embodiments of the invention.

In some embodiments, exemplary doses for two exemplary isotopes are illustrated, I-125 and Pd-103. Where, in some embodiments, each column, for each table, illustrates three exemplary embodiments, of different dose rates for three different distances from a surface of the exemplary source. In some embodiments, for each time duration after implantation, values of a column related to values of a similarly positioned column for the different time duration. For example, referring to the first example of the first column of the first table, an I-125 source, at one day post implantation irradiates at 0-1 mm 50 mGy/hour, at 5 mm 7.5 mGy/hour and, at 10 mm 1 mGy/hour and, in some embodiments, the same source irradiates at 30 days post implantation irradiates at 0-1 mm 37.5 mGy/hour, at 5 mm 5.625 mGy/hour and, at 10 mm, 0.75 mGy/hour. In some embodiments, all values within the table and/or within this section of the document are approximate values, understood to be +/−20% of the stated values.

### Additional Exemplary Embodiments

Patent document EP3136986 (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to structure/s as described in EP3136986.

FIG. 29A is a simplified schematic of an expandable device 2902, according to some embodiments of the invention.

FIG. 29B is a simplified schematic cross section of an expandable device 2902 within an aneurysm, according to some embodiments of the invention.

In some embodiments, expandable device 2902 includes an expandable body 2908 which in some embodiments, includes mesh e.g. is formed from mesh. Where, in some embodiments, an open end of a tubular mesh structure is closed by a connector 2966. In some embodiments, connector 2966 attaches a holder 2906 to expandable body 2908. In some embodiments, connector 2966 is connected to a delivery apparatus (e.g. including a push-wire), which, in some embodiments, is detached after deployment of the device. In some embodiments, holder 2906 hosts one or more radioactive source 2904. Alternatively, or additionally, in some embodiments, source 2904 is directly attached to expandable body 2098. In some embodiments, expandable body 2908 includes walls 2910 where, in some embodiments, walls 2910 conform, at least partially, to a shape of aneurysm 2926 inner walls. In some embodiments, expandable body 2908 includes a base portion 2916 (e.g. formed mesh) where, for example, walls 2910 are connected by base 2916. Where, in some embodiments, connector 2966 is connected to base portion 2916. In some embodiments, expansion of expandable body 2908 (e.g. elastic) in a direction away from connector and/or base holds device 2902 in position within aneurysm 2926, covering (at least partially) an opening of aneurysm 2926.

In some embodiments, device 2902 is symmetrical (e.g. in a relaxed expanded configuration) about one or more axis e.g. where a 3D geometry of the device, in some embodiments, is formed by rotating the cross section as illustrated in FIG. 29A around a central proximal-distal axis 2999.

Patent document US20190269414 (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to structure/s as described in US20190269414.

FIG. 30A is a simplified schematic of an expandable device 3002, according to some embodiments of the invention.

FIG. 30B is a simplified schematic cross section of an expandable device 3002 within an aneurysm 3026, according to some embodiments of the invention.

In some embodiments, device 3002 includes a plurality of expandable portions 3008 connected to each other by connectors 3066. In some embodiments, one or more of connectors 3066 FIG. 30A 3066a-d FIG. 30B (e.g. a most proximal of connectors 3066 e.g. 3066d FIG. 30B) are connected to a delivery apparatus (e.g. including a push-wire), which, in some embodiments is detached after deployment of the device. Where, in some embodiments, expandable portions include mesh e.g. are formed of mesh. In some embodiments, the mesh for each expandable portion has the same properties. Alternatively, in some embodiments, mesh of one or more of the expandable portions 3008 have different properties e.g. relaxed configuration dimension/s and/or porosity and/or mesh material. In some embodiments, individual expandable portions conform separately to a shape of aneurysm 3026. For example, each expandable portion 3008 changing length and/or width to fit aneurysm. In some embodiments, one or more portion of expandable device 3002 is connected to a source (e.g. exemplary sources and/or source attachment as described elsewhere in this document). Where, in some embodiments, one or more connector 3066 FIG. 30A, 3066a-d FIG. 30B is a radiation source and/or hosts one or more a radiation source and/or is attached to a holder which hosts one or more radiation source. In some embodiments, central connectors are and/or host radiation source/s e.g. connector 3066b and/or connector 3066c. In some embodiments, only connector/s located within a central region of device 3002 are and/or host sources.

U.S. Pat. No. 9,629,635 (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to structure/s as described in U.S. Pat. No. 9,629,635.

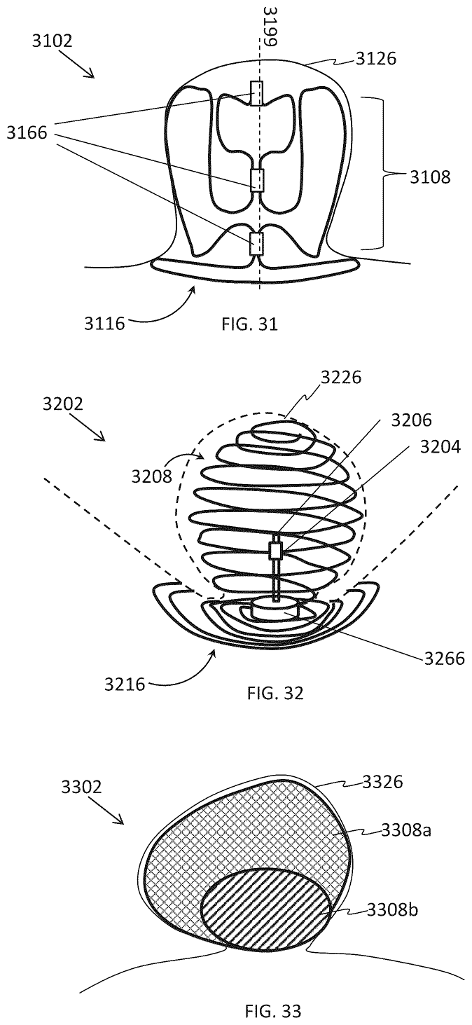

FIG. 31 is a simplified schematic cross section of an expandable device 3102 within an aneurysm 3126, according to some embodiments of the invention.

In some embodiments, expandable device 3102 includes an expandable body 3108 which is connected to a cover 3116. Where, in some embodiments, one or both of body 2108 and cover 3116 include (e.g. are formed of) mesh. In some embodiments, expandable body 3108 is expanded within aneurysm 3126 where, in some embodiments, sizing and/or relaxation force of the expandable body on the aneurysm walls holds body 3108 in position within aneurysm 3126. In some embodiments, cover 3116 is sized and/or shaped to cover an opening of aneurysm 3126. In some embodiments, cover 3116 is disposed outside aneurysm opening and/or is, when the device is correctly positioned e.g. as illustrated in FIG. 31 elastically relaxed. Alternatively, in some embodiments, cover 3116 is configured to at least partially conform to a shape of the blood vessel at a region of the aneurysm opening. Where, in some embodiments, cover as illustrated in FIG. 31 is applying expanding force on portion/s of the vessel wall with which it is in contact. In some embodiments, tension between connection of the cover and expandable body 3108 holds the cover against vessel tissue at the aneurysm opening, in positon. In some embodiments, device 3102 is symmetrical (e.g. in a relaxed expanded configuration) about one or more axis e.g. where a 3D geometry of the device, in some embodiments, is formed by rotating the cross section as illustrated in FIG. 31 around a central proximal-distal axis 3199. In some embodiments, device 3102 includes one or more connector 3166. Where, in some embodiments, connector/s 3166 (e.g. a most proximal of connector/s 3166) close open region/s of tubular mesh and/or hold a shape (e.g. including folded portion/s) of device 3102 in position. In some embodiments, connector/s 3166 are connected to a delivery apparatus (e.g. including a push-wire), which, in some embodiments is detached after deployment of the device. In some embodiments one or more of connector/s is a radioactive source (e.g. as described within this document) and/or hosts a radioactive source (e.g. externally and/or within a lumen) and/or is attached to a holder which hosts one or more radioactive source. In some embodiments, only connector/s located within a central region of device 3102 are and/or host sources.

Patent document US20170367708 (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to structure/s as described in US20170367708.

FIG. 32 is a simplified schematic view of an expandable device within an aneurysm, according to some embodiments of the invention.

In some embodiments, device 3202 includes (e.g. is formed of) an elongated portion (e.g. wire) which, when in a relaxed configuration coils through space to delineate a shape of the device 3202. In some embodiments, device 3202 includes a body 3208 and a cover 3216 where body and/or cover include one or more feature as illustrated in and/or described regarding cover 3216 and/or body 3208. In some embodiments, device 3216 includes a connector 3266. Where, in some embodiments, connector 3266 connects cover 3216 to body 3208. In some embodiments, connector 3266 is connected to a delivery apparatus (e.g. including a push-wire), which, in some embodiments is detached after deployment of the device. In some embodiments, connector 3266 includes one or more radioactive source 3204. Alternatively or additionally, in some embodiments, connector 3266 hosts one or more source 3204. In some embodiments, connector 3266 is connected to a holder 3206 hosting source 3204. In some embodiments, for example, where connector 3266 is radiopaque, a source is connected proximally to connector 3266, optionally, by a holder.

Patent document US20180140305 (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to structure/s as described in US20180140305.

FIG. 33 is a simplified schematic view of an expandable device 3302 within an aneurysm 3326, according to some embodiments of the invention.

In some embodiments, expandable device 3302 includes an outer body 3308a. Where, in some embodiments, outer body 3308a expands to conform to a shape of aneurysm 3326. In some embodiments, outer body 3308a includes (e.g. is formed of) mesh. Optionally, in some embodiments, device 3302 includes an inner body 3308b. In some embodiments, inner body 3308b is positioned to provide a double layer of mesh at an opening of aneurysm 3326. In some embodiments, inner body 3308b is attached to outer body 3308b e.g. to position inner body 3308b at the aneurysm opening. In some embodiments, inner body 3308b is delivered and/or expanded separately to outer body 3308b.

In some embodiments, one or more portion of outer body 3308a and/or of inner body 3308b is a radioactive source. In an exemplary embodiment, inner body 3308b includes a radioactive source e.g. in some embodiments, mesh of inner body 3308b (e.g. of the entire inner body 3308b, in some embodiments) is a radioactive source. For example, as described elsewhere in this document, in some embodiments, a portion of a body of a radioactive device is itself a radioactive source e.g. at least a portion of a mesh e.g. in some embodiments is treated to become a radioactive source, the emissions of which are according to description of sources elsewhere in this document. In some embodiments, a portion of a mesh body is a radioactive source e.g. where treatment includes one or more feature of treatment's as described elsewhere in this document.

Patent document JP2019526324A (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to structure/s as described in JP2019526324A and/or are attached to commercially available devices for treatment of LAA for example, device/s produced by Boston Scientific e.g. Watchman™.

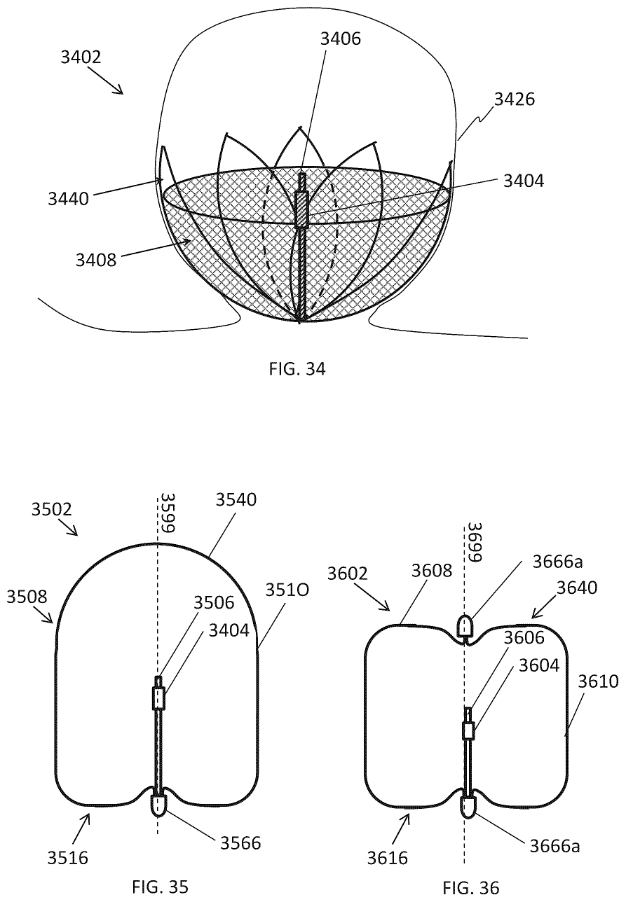

FIG. 34 is a simplified schematic view of an expandable device 3402 within an aneurysm 3426, according to some embodiments of the invention.

In some embodiments, an open ended device 3402 e.g. where positioning and/or maintaining of the device in position includes one or more feature as described regarding open ended device 2902 FIGS. 29A-B. In some embodiments, device 3402 includes a support structure 3440. In some embodiments, support structure 3440 is expandable e.g. elastically expandable. In some embodiments, support structure 3440 is formed from elongated element/s e.g. wires. In some embodiments, support structure is at least partially covered in a body 3408. Where, in some embodiments, body 3408 includes (e.g. is formed of) mesh. Alternatively or additionally, in some embodiments, body 3408 includes a polymer layer. In some embodiments, at least portion of the body 3408 includes radioactive material, for example, being a source. Alternatively or additionally, in some embodiments, a source 3404 (or multiple sources) are attached to support 3440 and/or body 3408 directly and/or via a holder 3406 hosting source 3404.

In some embodiments, device 3402 includes a distal support structure (not illustrated) including one or more feature of distal support structure 1682 as illustrated in and/or described regarding FIGS. 16A-B.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to commercially available aneurysm treatment device/s produced by Medtronic e.g. ARTISSE™.

FIG. 35 is a simplified schematic cross section of an expandable device 3502, according to some embodiments of the invention.

In some embodiments, device 3502 is symmetrical (e.g. in a relaxed expanded configuration) about one or more axis e.g. where a 3D geometry of the device, in some embodiments, is formed by rotating the cross section as illustrated in FIG. 35 around a central proximal-distal axis 3599.

In some embodiments, device 3502 includes an expandable body 3508 which includes one or more feature as described and/or illustrated regarding expandable bodies elsewhere in this document. In some embodiments, expandable body 3508 includes walls 3510 (e.g. tubular walls 3510) and a base 3516. In some embodiments, expandable body 3508 includes (e.g. is formed of mesh). In some embodiments, expandable body 3508 includes a top 3540 where a mesh structure of the top is not open and/or not closed with a connector. Optionally, in some embodiments, mesh ends meeting at a central region of base 3516 are connected by a connector 3566. In some embodiments, connector 3566 connects a detachable delivery apparatus (not illustrated) to device 3502. In some embodiments, one or more radioactive source 3504 is connected to expandable body 3508, for example, by connector 3566 directly and/or via a holder 3506. Where, in some embodiments, holder 3506 includes one or more feature as illustrated in figures associated with of this document and/or described regarding holder/s, elsewhere in this document.

U.S. Pat. No. 9,629,635 (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) are attached to device/s as described in U.S. Pat. No. 9,629,635 and/or are attached to commercially available devices for treatment of aneurysm for example, device/s produced by Terumo Group e.g. Woven EndoBridge® WEB™ SL and/or WEB™ SLS.

FIG. 36 is a simplified schematic cross section of an expandable device 3602, according to some embodiments of the invention.

In some embodiments, device 3602 includes an expandable body 3608 having tubular walls 3610 where ends of the walls, proximally and distally are connected to form a base 3616 and a top 3640 respectively, by connectors 3666a and 3666b respectively. In some embodiments, device 3602 is symmetrical (e.g. in a relaxed expanded configuration) about one or more axis e.g. where a 3D geometry of the device, in some embodiments, is formed by rotating the cross section as illustrated in FIG. 36 around a central proximal-distal axis 3699.

In some embodiments, proximal connector 3666a connects a detachable delivery apparatus (not illustrated) to device 3602. In some embodiments, one or more radioactive source 3604 is connected to expandable body 3608, for example, by one of connector/s 3666a 3666b (and in an exemplary embodiment, by proximal connector 3666b) directly and/or via a holder 3606. Where, in some embodiments, holder 3606 includes one or more feature as illustrated in figures associated with of this document and/or described regarding holder/s, elsewhere in this document.

U.S. Pat. No. 9,844,382 (which is herein incorporated by reference in its entirety) shows vascular deformity (e.g. aneurysm) treatment devices.

In some embodiments, exemplary source/s as described in this document (e.g. in the section titled “Exemplary radiation levels” and/or in the section titled “Exemplary sources and exemplary source attachment”) are attached (e.g. including one or more attachment method and/or structure as described in the section of this document titled “Exemplary sources and exemplary source attachment”) to structure/s as described in U.S. Pat. No. 9,844,382.

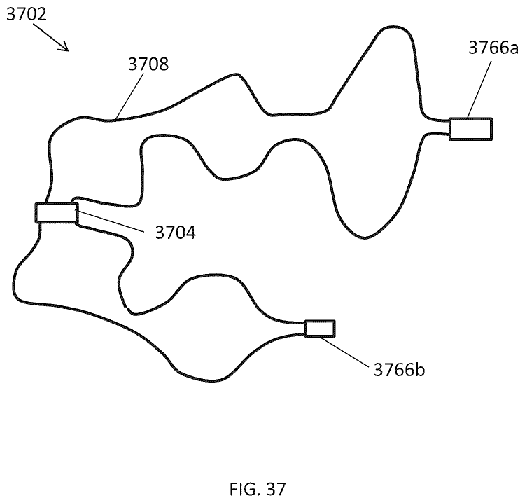

FIG. 37 is a simplified schematic cross section of an expandable device 3702, according to some embodiments of the invention.

In some embodiments, expandable device 3702 is an elongate structure, including a mesh body 3708. Where, in some embodiments, a body 3708, which in some embodiments, has a tubular structure, is coiled into an aneurysm. In some embodiments, mesh body 3708 is expandable at a direction perpendicular to a central longitudinal axis of the device, at one or more region. Where, for example, in some embodiments, once within an aneurysm, expandable regions expand e.g. increasing a volume of the aneurysm which is enclosed by surfaces of the device. In some embodiments, one or more portion of the elongate structure includes a connector 3766a, 3766b which in some embodiments, hosts a source and/or is connected to a holder hosting a source. Alternatively or additionally, in some embodiments one or more source 3704 is connected to body 3708 along a length of the body. In embodiments, where a source is connected to one or both of connectors 3766a, 3766b, source 3704 is optional.

### Additional Exemplary Methods

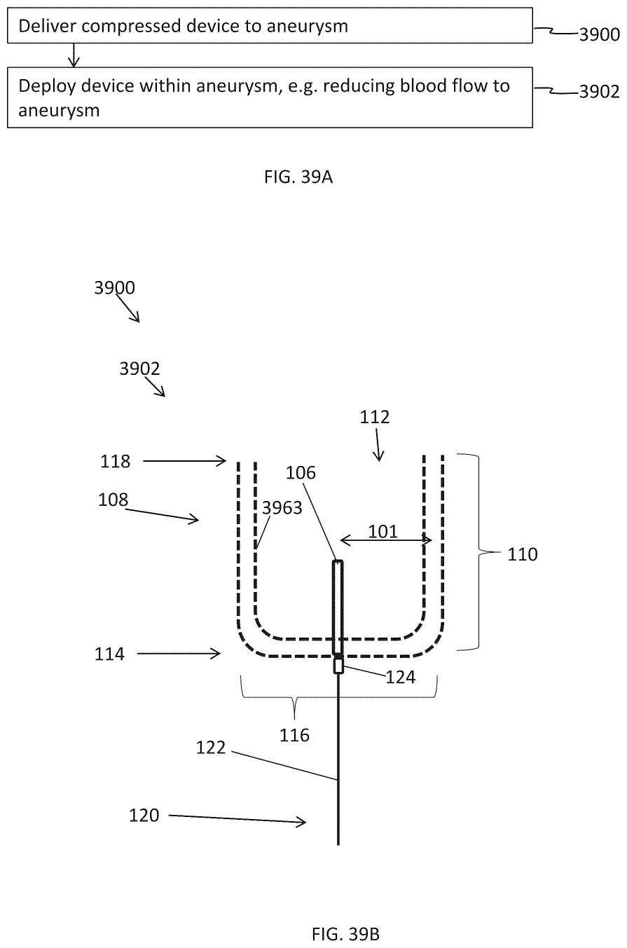

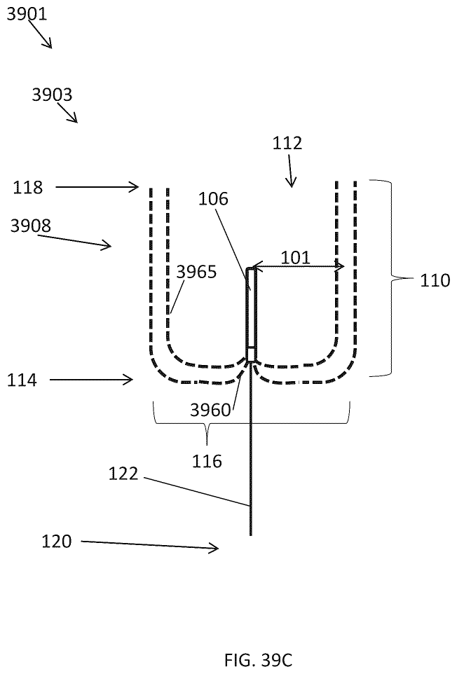

FIG. 39A is a flow chart of a method, according to some embodiments of the invention.

At 3900, in some embodiments, a compressed device is delivered to a treatment site, e.g. to an aneurysm. Delivery, in some embodiments, including one or more feature of step 208 FIG. 2.

In some embodiments, the device does not include radioactive source/s. For example, devices as described elsewhere in this document are used without attachment of a radioactive source. In some embodiments, the device does not include a holder for a radioactive source. Where, in some embodiments, holders as described elsewhere within this document are lacking and/or replaced by element/s offering the described mechanical feature/s.

In some embodiments, the device includes one or more feature of device 102 of FIG. 1 without source 104 and/or holder 106.

In some embodiments, the device includes one or more feature of device 402 of FIGS. 4A-H without source 404 and/or holder 406. For example, in some embodiments, device 402 without holder 406, push-wire 422 is attached directly to body 408 e.g. as opposed to attached to holder 406 which provides attachment of the push-wire 422 to body 408.

In some embodiments, the device includes one or more feature of device 502 of FIG. 5 without source 504 and/or holder 506.

In some embodiments, the device includes one or more feature of device 602 of FIG. 6 without source 604 and/or holder 606.

In some embodiments, the device includes one or more feature of device 702 of FIG. 7 without source 704 and/or holder 706.

In some embodiments, the device includes one or more feature of device 802 of FIG. 8 without source 804 and/or holder 806.

In some embodiments, the device includes one or more feature of device 902 of FIG. 9 without source 904 and/or holder 906.

In some embodiments, the device includes one or more feature of device 1002 of FIG. 10 without source 1004 and/or holder 1006.

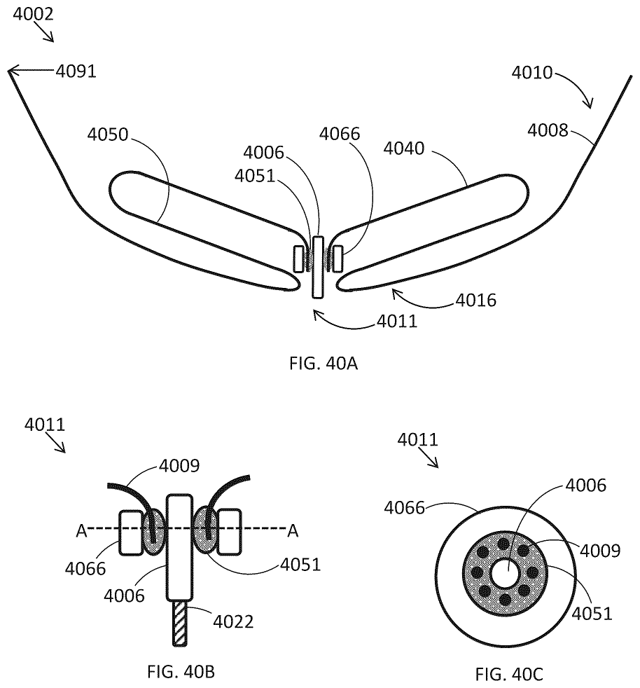

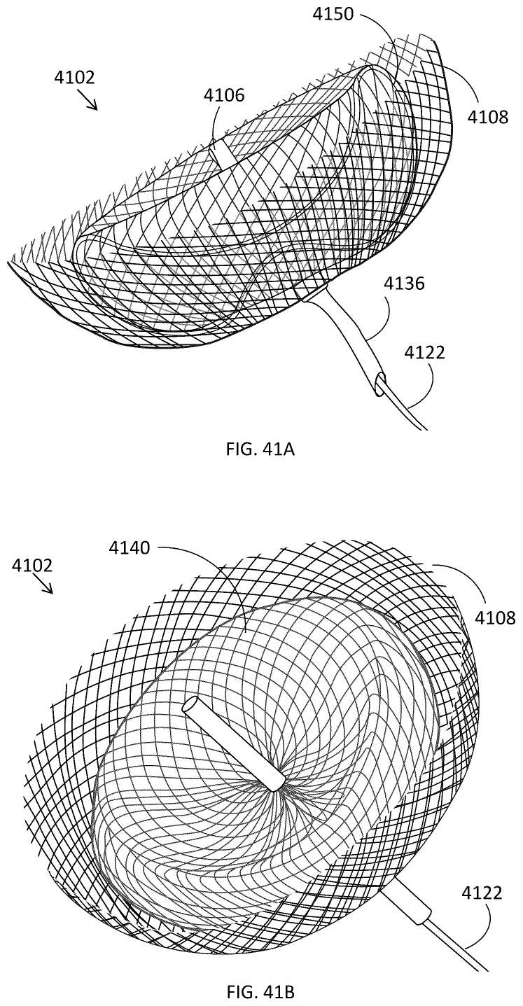

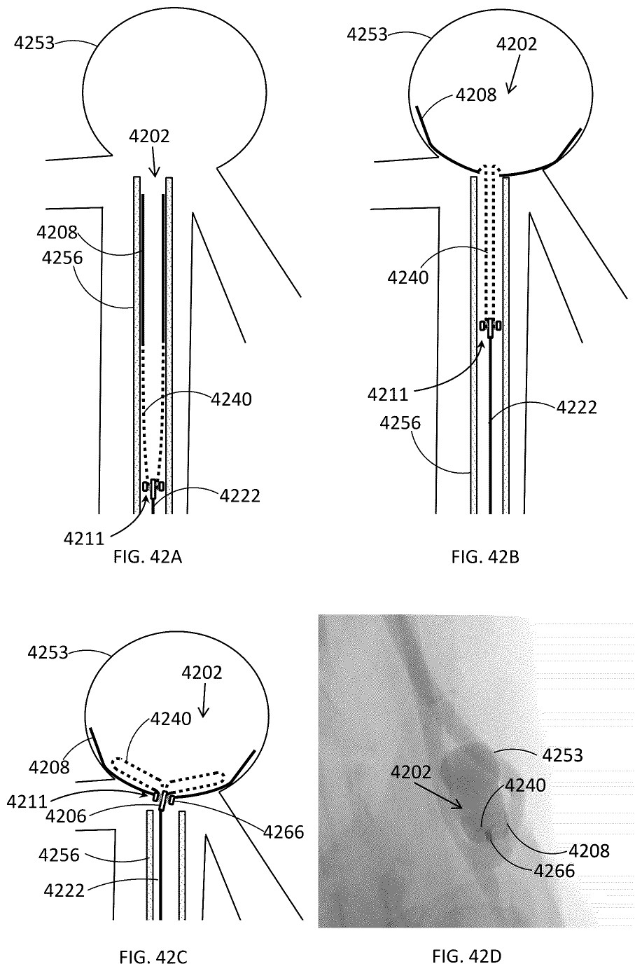

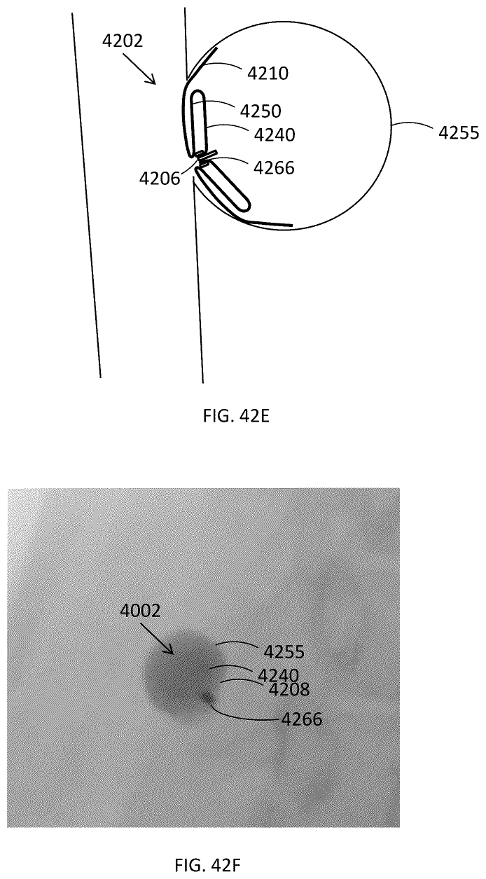

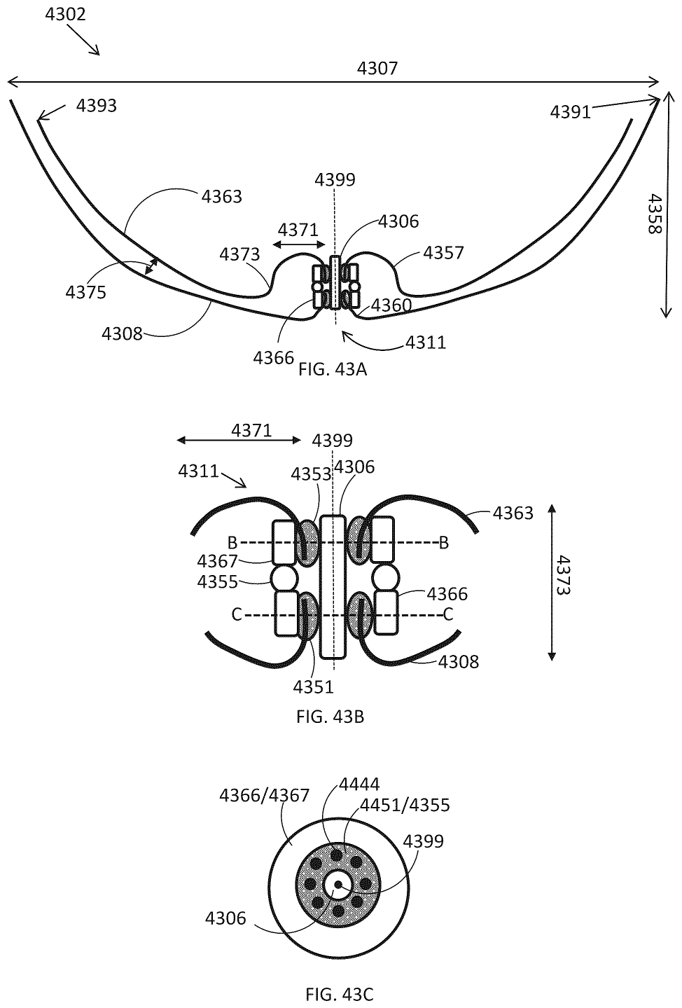

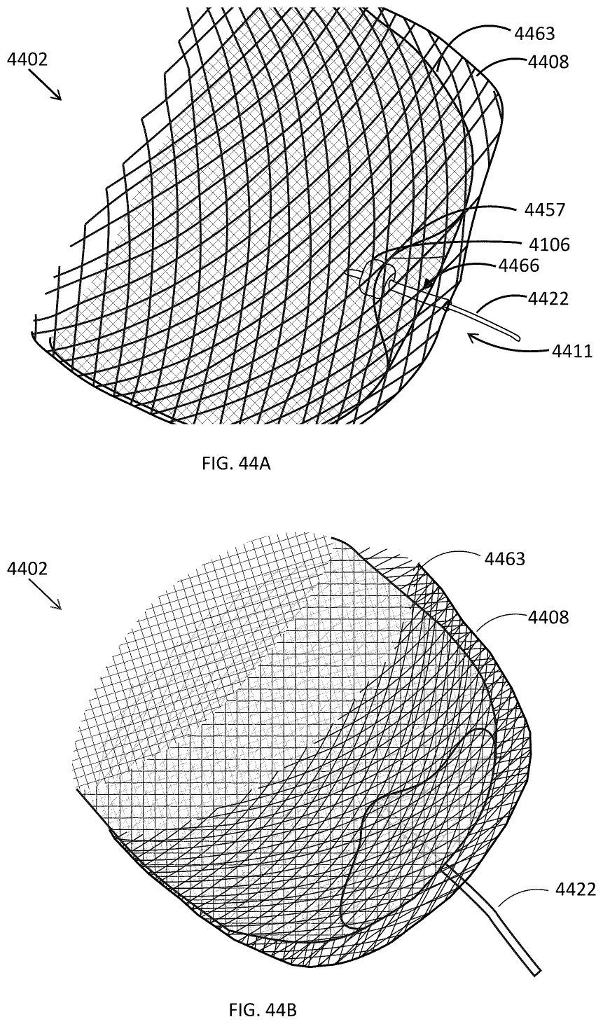

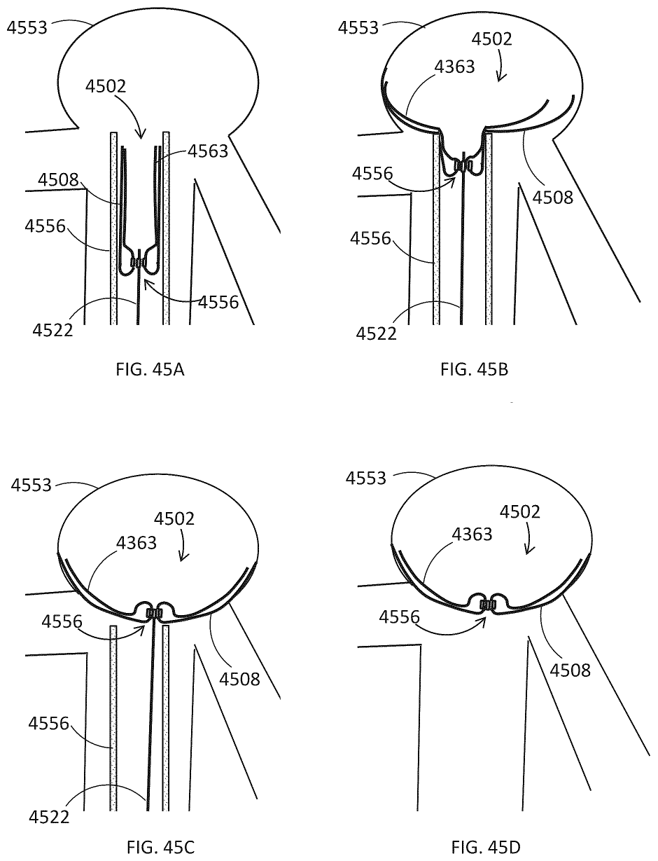

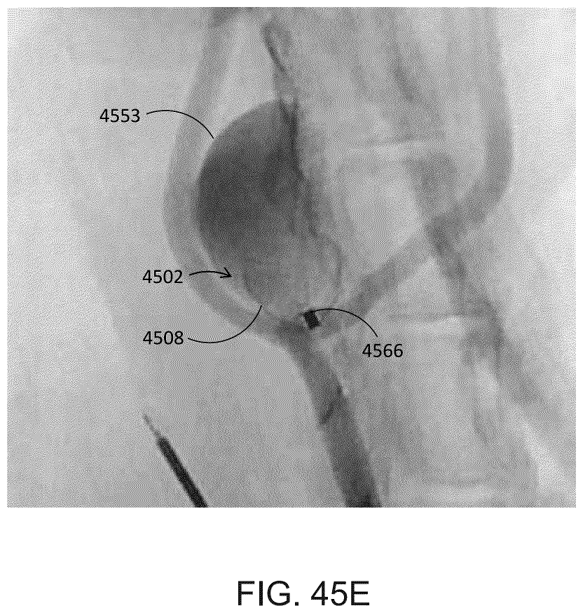

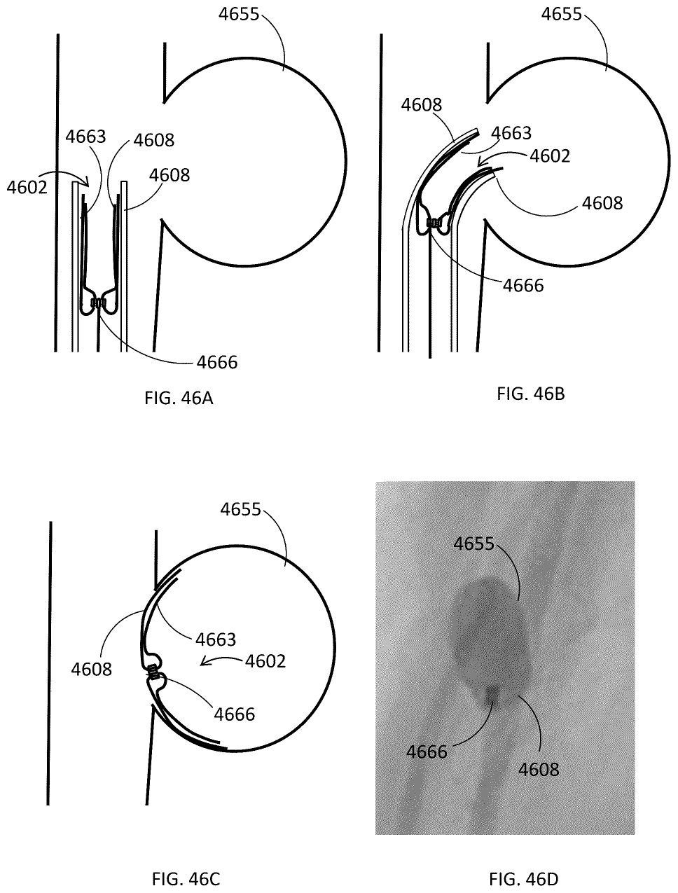

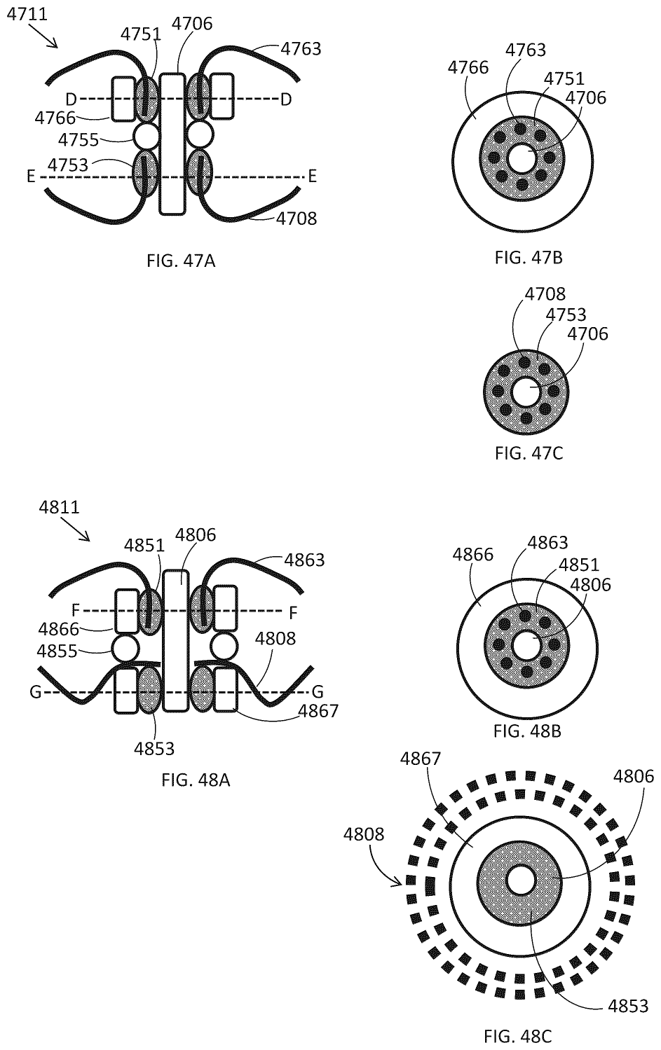

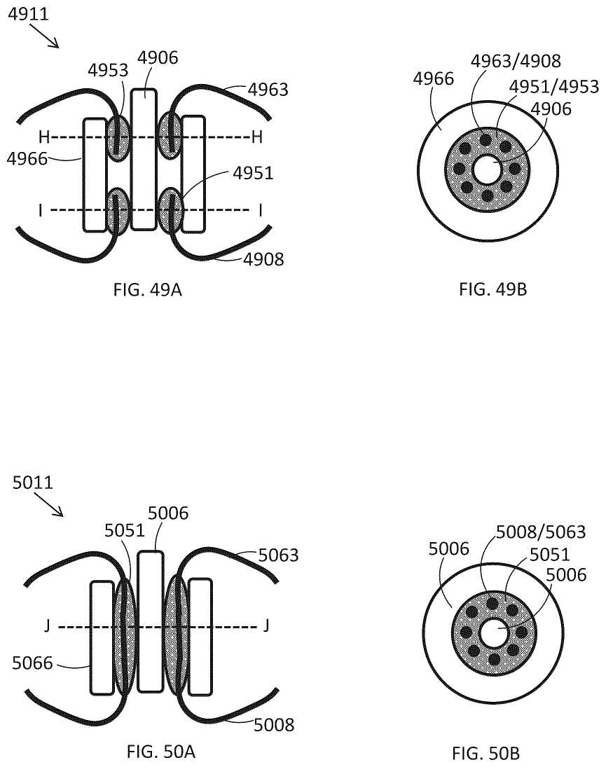

In some embodiments, a device includes one or more feature of device 1102 of FIG. 11 where no source is placed within lumen 1146 and/or where device 1102 lacks portion/s 1136 and/or 1134. For example, connection to a push-wire being provided by portion/s replacing portion 1136 and/or portion 1134. For example, in some embodiments, a device has shape and/or geometry and/or other body characteristic/s as described regarding body 1108 but with an attachment region including feature/s as described elsewhere in this document e.g. regarding one or more of attachment regions 4011 FIGS. 40A-C, 4111 FIGS. 41A-B, 4211 FIGS. 42A-F, 4311 FIGS. 43A-C, 4411 FIGS. 44A-B, 4511 FIGS. 45A-D, 4611 FIGS. 46A-D, 4711 FIGS. 47A-C, 4811 FIGS. 48A-C, 4911 FIGS. 49A-B, 5011 FIGS. 50A-B.

In some embodiments, the device includes one or more feature of device 1202 of FIGS. 12A-H e.g. without source 1204 (and/or holder 1206).

In some embodiments, the device includes one or more feature of device 1302 (e.g. having shape and/or geometry and/or other characteristics of body 1108) of FIGS. 13A-C without a source and/or holder 1306.

In some embodiments, the device includes one or more feature of device 1402 (e.g. having shape and/or geometry and/or other characteristics of body 1408) of FIG. 14A-C without a source and/or holder 1406.

In some embodiments, the device includes one or more feature of device 1502 (e.g. having shape and/or geometry and/or other characteristics of body 1508) of FIGS. 15A-C without a source and/or holder 1506.

In some embodiments, the device includes one or more feature of device 1602 (e.g. having shape and/or geometry and/or other characteristics of body 1608) of FIGS. 16A-B without source 1604 and/or holder 1606.

In some embodiments, the device includes one or more feature of device 1702 (e.g. having shape and/or geometry and/or other characteristics of body 1708) of FIGS. 17A-B without a source and/or holder 1706.

In some embodiments, the device includes one or more feature of device 1802 (e.g. having shape and/or geometry and/or other characteristics of a body of device 1802 of FIG. 18 without a source and/or holder 1806 (device body e.g. including base 1816 and/or walls 1810 and/or support base 1884 and/or walls 1886).

In some embodiments, the device includes one or more feature of device 2702 (e.g. having shape and/or geometry and/or other characteristics of body 2708) of FIG. 27 without source 2704a and/or holder 2706.

In some embodiments, the device includes one or more feature of device 2802 (e.g. having shape and/or geometry and/or other characteristics of body 2808) of FIG. 28 without sources 2804a-d and/or holder 2806.

At 3902, in some embodiments, the device is deployed at the treatment site. For example, deployed into the aneurysm. Deployment, for example, including one or more feature of step 210 of FIG. 2.

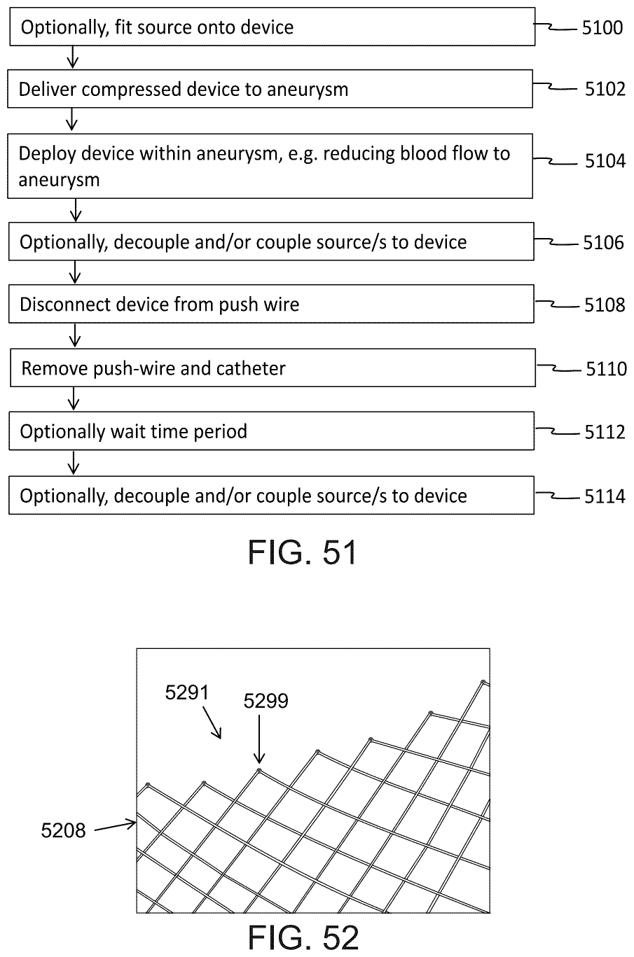

FIG. 51 is a flowchart of a method, according to some embodiments of the invention.

At 5100, optionally, a radioactive source is fitted onto a device. Fitting, for example, including one or more feature of step 202 FIG. 2.

At 5102, in some embodiments, a compressed device is delivered to a treatment region (e.g. to an aneurysm). For example, delivery including one or more feature as described regarding step 3900 FIG. 39A.

In some embodiments, the device is compressed by inserting the device into a tubular structure e.g. crimping tube. For example, by threading the device through the crimping tube (e.g. using the push-wire) and then pulling the device into the crimping tube e.g. by pulling on the device push-wire.

At 5104, in some embodiments the device is deployed within the treatment site (e.g. aneurysm). For example, deployment including one or more feature as described regarding step 3902 FIG. 39A.

At 5106, optionally, in some embodiments, one or more radioactive source is decoupled and/or coupled to the device.

For example, in some embodiments, once deployed, radioactive source/s are delivered to the treatment region and optionally coupled and/or attached to the deployed device. In some embodiments, after device deployment, portion/s of the device for hosting radioactive source/s are selected e.g. based on their position with respect to treatment site anatomy. For example, in some embodiments, once deployed radioactive source/s are decoupled from the device e.g. and then are removed or repositioned. A potential benefit of coupling and/or decoupling after deployment being the ability to accurately position source/s with respect to treatment site anatomy.

In some embodiments, after device deployment, a medical practitioner decides whether or not to couple or decouple radioactive source/s. For example, after conducting one or more measurement. For example, in some embodiments, flow measurement/s are collected after positioning of the device and, based on how the mechanical structure of the expanded device affects blood flow to the aneurysm, radioactive sources are coupled and/or decoupled. For example, where blood flow is sufficiently reduced mechanically, in some embodiments, no radioactive source is coupled, the device, in situ, in some embodiments, not including any radioactive sources.

At 5108, in some embodiments, the device is decoupled from delivery apparatus e.g. from a push-wire. For example, including one or more feature of step 212 FIG. 2.

At 5110, in some embodiments, delivery apparatus (e.g. including the push-wire and a delivery catheter) is removed. For example, including one or more feature of step 214 FIG. 2.

At 5112, optionally, in some embodiments, a time period is allowed to elapse. For example, sufficient time to determine treatment efficacy of the device in situ. For example, the time period being 1 day to 6 months. In some embodiments, imaging and/or other measurement/s are performed to determine efficacy of treatment with the device (e.g. the patient is diagnosed using one or more feature of step 200 FIG. 2). In some embodiments, based on the diagnosis, change/s to the device are selected. Where, in some embodiments, change/s include removal and/or addition of radioactive source/s.

At 5114, optionally, in some embodiments, decouple and/or couple radioactive source/s to device. For example, as selected based on the diagnosis. In some embodiments, a delivery apparatus is positioned at the treatment site and interacts with the device to couple and/or decouple one or more radioactive source.

For example, referring to FIGS. 11, 19A-B, where the device includes container/s configured to house radioactive device/s coupled to the device, in some embodiments, a container is opened by the delivery apparatus to remove a radioactive source or to insert a radioactive source (in some embodiments, to insert an additional radioactive source).

In some embodiments, one or more radioactive source is attached to the device e.g. to a holder of the device and/or to a body of the device. For example, where, in some embodiments, a radioactive source is wrapped (or unwrapped) around a portion of the device (e.g. holder) and/or fixed onto a portion of the device (e.g. a coil radioactive source is pushed onto (or pulled off of) a portion of the device e.g. holder.)

### Additional Exemplary Expandable Devices

FIG. 39B is a simplified cross sectional schematic of an intravascular device system 3900, according to some embodiments of the invention.

In some embodiments, system 3900 includes one or more feature of system 100 FIG. 1 but lacking source 104 FIG. 1 and/or device 3902 includes one or more feature of device 102 of FIG. 1 but lacking source 104 FIG. 1.

In some embodiments, device 3902 includes a second body layer 1363 (also termed “support”).

FIG. 39C is a simplified cross sectional schematic of an intravascular device system 3901, according to some embodiments of the invention.

In some embodiments, system 3901 includes one or more feature of system 100 FIG. 1 but lacking source 104 FIG. 1 and/or device 3903 includes one or more feature of device 102 of FIG. 1 but lacking source 104 FIG. 1.

In some embodiments, device 3904 includes a second body layer 3965 (also termed “support”).

In some embodiments, a device body 3908 is recessed e.g. at a base, a recessed portion 3960 including one or more feature of recessed portion 1360 FIGS. 13A-B, and/or recessed portion 1460 FIGS. 14A-C, and/or recessed portion 1560 FIGS. 15A-C, and/or recessed portion 1788 FIGS. 17A-B.

FIG. 40A is a simplified schematic cross sectional view of a device 4002, according to some embodiments of the invention.

FIG. 40B is a simplified schematic cross sectional view of a portion 4011 of a device, according to some embodiments of the invention.

In some embodiments, FIG. 40B shows an enlarged view of connection region 4011 of FIG. 40A.

FIG. 40C is a simplified schematic cross sectional view of a portion 4011 of a device, according to some embodiments of the invention.

In some embodiments, FIG. 40C illustrates a cross section of portion 4011 taken along line AA of FIG. 40B.

In some embodiments, device 4002 includes one or more feature of device/s 1202 as illustrated and/or described regarding FIGS. 12A-H. Where, in some embodiments, like numerals refer to corresponding features, e.g. feature 1210 corresponding to feature 4010.

In some embodiments, device 4002 includes an expandable body 4008. In some embodiments, body 4008 has a curved cup shape including wall regions 4010 and a base region 4016.

In some embodiments, at least a portion of expandable body 4008 is constructed by using a tubular shaped material (e.g. mesh) where one of the ends of the tubular shape is narrowed and/or closed and/or is held together at a connection region 4011. The tubular shape, in some embodiments, has a lower cross sectional area at an end of the device at the connection region 4011 than that at the distal ends of walls 4010. In some embodiments, the tubular shape has reducing cross sectional area moving in a direction towards the connection region 4011 e.g. to form a flattened cup shape with an opening at connection region 4011.

In some embodiments, the tubular mesh has a uniform porosity and/or mesh density along the tube. In some embodiments, the tubular mesh is then shaped e.g. folded and/or constricted (e.g. attached at attachment region) to form body 4008 and/or support 4040. In some embodiments, folding and/or constriction changes porosity of the device and/or density of mesh in one or more portion of the device e.g. from that of the tubular structure from which the device is formed. For example, as illustrated in FIGS. 41A-B where mesh density increases (e.g. porosity decreases) towards a center of the device. In some embodiments, the device is elastically relaxed when in an open configuration as illustrated in FIGS. 40A-C and/or FIGS. 41A-B. In some embodiments, elastic relaxation in the open shape configuration is produced using a tubular nitinol structure which is heat treated to have shape memory of the open configuration. For example, in some embodiments, a tubular mesh is inserted into and/or positioned on a mold and then heat treated to shape memory set at least a portion of the shape of the device to that of the mold. In some embodiments, shape of the device is set using a single mold and/or heat treatment. In some embodiments, a plurality of mold/s and/or heat treatments are used (e.g. sequentially) to shape the device. In some embodiments, device 4002 includes one or more support 4040, 4050. Where, in some embodiments, support 4040, 4050 includes one or more additional layer of material (e.g. body material) which layer/s at least extend over (at least a portion of) a central region (e.g. at least a central 10%, e.g. central in at least one dimension) of the device. Where, in some embodiments, a central region of the device is at connection region 4011.

In an exemplary embodiment, support 4040, 4050 is formed from the same tube of material (e.g. mesh) as body 4010. Support portions 4040, 4050 being formed by folding of the tube one or more times e.g. twice as illustrated in FIG. 40A.

In some embodiments, device 4002 optionally includes a holder 4006 and/or a optionally includes a connector 4066 and/or optionally includes bonding material 4051.

In some embodiments, connection region 4011 (e.g. including one or more of holder 4006, connector 4066, and bonding material) provides one or more of connection of body end portion 4009 (e.g. as described above), connection of device 4002 to a delivery apparatus 4022, connection of device 4002 to a holder 4006, radiopaque marking (e.g. in some embodiments connector 4066 and/or holder 4006 include radiopaque material), and, optionally, hosting of radioactive source/s.

In some embodiments, of illustrated elements holder 4006, connector 4066, and bonding material 4051, device 4002 includes only bonding material. Where, for example, end portion 4009, and/or delivery apparatus 4022 are attached by bonding and/or welding only 4051.

In some embodiments, along with bonding material 4051, connector 4066 is present. For example, connector 4066 providing mechanical support to connection of end portion 4009 and/or radiopaque marking.

In some embodiments, device 4002 also includes a holder 4006.

Optionally, in some embodiments, holder 4006 is disconnected from delivery apparatus 4022 during and/or after device deployment. In some embodiments, holder 4006 is a portion of the delivery apparatus and, in some embodiments, is removed from device 4002 during and/or after device deployment.

In some embodiments, connection of body 4008 at connection region 4011 is by bonding and/or welding another portion to a portion of body 4008 end. For example, by bonding one portion to body 4008 end. For example, by bonding one of holder 4006 and connector 4066 to body e.g. where, the other of holder 4006 and connector 4066 is not connected (e.g. is removable) and/or is not present.

Referring now to FIGS. 40B-C, in some embodiments, connection is by holding a body end portion 4009 between two other portions (which end portion 4009, in some embodiments, is an end portion of the tubular material extending from support 4040). For example, in some embodiments, end portion 4009 is held between a connector 4066 and a holder 4006, optionally with bonding material 4051 disposed between the connector and holder. In some embodiments, for example, where body 4008 material is porous (e.g. is a mesh), end portion 4009 material is immersed in bonding material 4051 e.g. as illustrated in FIG. 40B. In some embodiments, bonding material 4051 is one of disposed between connector 4066 and body portion 4051 and disposed between body 4008 and holder 4006.

In some embodiments, for example, as described elsewhere in this document, body portion/s of device 4002 are connected and/or closed at connection region 4011.

In some embodiments, holder 4006 (e.g. as described elsewhere in this document) holds one or more radioactive source and/or provides connection to delivery and/or positioning apparatus 4122.

In some embodiments, body 4008 is bonded to holder 4006 and/or to a connector 4066 e.g. by bonding material 4051. In some embodiments, connector 4066 provides mechanical strength to connection of body 4008 material. Alternatively or additionally, connector 4066 is a marker e.g. includes radiopaque material.

In some embodiments, connector 4066 is annular (e.g. as illustrated in FIG. 40C, FIG. 40B and FIG. 40C together illustrating, in some embodiments, where connector 4066 is cylindrical). In some embodiments, bonding material 4051 is continuous in a contour around connector 4066 and/or holder 4006 (e.g. as illustrated in FIG. 40C). Alternatively, connector 4066 and/or bonding material 4051 form a non-continuous contour e.g. for one or more cross section in the direction of FIG. 40C.

In some embodiments, bonding material 4051 includes adhesive e.g. epoxy e.g. UV cured epoxy. In some embodiments, bonding material 4051 includes weld.

In some embodiments, body 4010 is porous e.g. includes a mesh. For example, FIG. 40C illustrating body 4010 porosity where bonding material 4051, in some embodiments, enters into pores of body 4010.

In some embodiments, device body 4008 is disconnected from delivery apparatus (e.g. push-wire 4022) e.g. after deployment e.g. where disconnecting, in some embodiments, includes one or more feature as illustrated in and/or described regarding detachment zone 2392 FIG. 23.

FIG. 41A is an image of a side view of a device 4102, according to some embodiments of the invention.

FIG. 41B is an image of a device 4102, according to some embodiments of the invention.

In some embodiments, device 4102 includes one or more feature of device 4002 FIGS. 40A-C and/or device 1202 FIGS. 12A-F.

Visible in FIGS. 41A-B are a device body 4180, a support 4140, and a push-wire 4122. In some embodiments, holder 4106 includes electrically insulating material (e.g. PEEK). In some embodiments, for example, as described regarding detachment region 2392 FIG. 23, push-wire 4122 is detached from device 4102 using applied electrical stimulation. In some embodiments, a holder proximal portion 4136 extends proximally outwards from body 4108 and, optionally, in some embodiments, includes electrically and/or thermally insulating material (e.g. PEEK). In an exemplary embodiment, holder 4106 is formed by covering and/or coating portion/s of push-wire 4122 with electrically insulating material (e.g. PEEK).

FIG. 42A is a simplified schematic cross sectional view of a device 4202 being delivered to a treatment region 4253, according to some embodiments of the invention.

FIG. 42B is a simplified schematic cross sectional view of a device 4202 being deployed in a treatment region 4253, according to some embodiments of the invention.

FIG. 42C is a simplified schematic cross sectional view of a deployed device 4202 in a treatment region 4253, according to some embodiments of the invention.

In some embodiments, FIGS. 42A-F illustrate delivery, and deployment of the same device. In some embodiments, device 4202 includes one or more feature of device 1202 FIGS. 12A-F, and/or device 4002 FIGS. 40A-C, and/or device 4102 FIGS. 41A-B.

In some embodiments, a device body 4008 and a support 4240 are formed with a single tubular structure where illustration of body 4008 using solid lines and support 4240 using dashed lines, in some embodiments, is to illustrate portions of the tubular structure which, when the device is in an expanded configuration, correspond to the body 4008 and support 4240.

Referring now to FIG. 42A, in some embodiments, a delivery catheter 4256 is positioned with an outlet at the treatment region 4253 e.g. where the outlet is opposite an opening of an bifurcation aneurysm 4253. FIG. 42A, in some embodiments, illustrates device 4202 in a collapsed and/or crimped configuration. Where, in some embodiments, device 4202 is stretched from the elastically relaxed open configuration into a tubular shaped where an end of the tubular shape is closed at a connection region 4211. In some embodiments, the end is connected to a push-wire 4222. A potential advantage of the crimped configuration illustrated in FIG. 42A is low profile (e.g. cross sectional area in a direction perpendicular to the cross section illustrated in the figure) of the crimped device, for example, potentially enabling use of a smaller cross section catheter, navigation of tortuous vasculature and/or deployment through small cross section vasculature. In some embodiments, a device collapsed and/or crimped configuration maintains folding of support 4040 e.g. as illustrated in FIG. 12E.

In some embodiments, the device is advanced through catheter 4256 by pushing pressure applied to push-wire 4222.

Referring now to FIG. 42B, in some embodiments, as body 4208 of device 4202 exits catheter it elastically relaxes body 4208 forming a curved closure to aneurysm 4253.

Referring now to FIG. 42C, in some embodiments, as device 4202 is fully delivered through catheter 4256 support 4240 elastically relaxes to fold into position within a volume defined by body 4208 (e.g. by a base and walls of the body).

In some embodiments, after device 4202 is deployed, e.g. as illustrated in FIG. 42C, push-wire 4222 is detached from device 4202 and withdrawn e.g. after and/or along with delivery catheter 4256. In some embodiments, holder 4206 is detached from device 4202 e.g. and withdrawn along with push-wire 4222. In some embodiments, holder 4206 is a portion of push-wire 4222.

FIG. 42D is an image of a deployed device 4202 in a treatment region 4253, according to some embodiments of the invention.

In some embodiments, FIG. 42D illustrates an x-ray image of deployed device 4202 within a bifurcation aneurysm 4253. Visible in FIG. 42D are device body 4208, support structure 4240, and marker 4266. In FIG. 42D, delivery apparatus (e.g. including a push-wire and/or a catheter) has been withdrawn.

FIG. 42E is a simplified schematic cross sectional view of a deployed device 4202 in a treatment region 4255, according to some embodiments of the invention.

In some embodiments, treatment region 4255 is a saccular aneurysm. In some embodiments, delivery and/or deployment to a saccular aneurysm of device 4202 includes one or more feature as described regarding delivery and/or deployment of device 4602 FIGS. 46A-C e.g. bending of a delivery catheter.

FIG. 42F is an image of a deployed device 4202 in a treatment region 4255, according to some embodiments of the invention.

In some embodiments, FIG. 42F illustrates an x-ray image of deployed device 4202 within a saccular aneurysm 4255. Visible in FIG. 42F are device body 4208, support 4240, and marker 4266. In FIG. 42F, delivery apparatus (e.g. including a push-wire and/or a catheter) has been withdrawn.

FIG. 43A is a simplified schematic cross sectional view of a device 4302, according to some embodiments of the invention.

FIG. 43B is a simplified schematic cross sectional view of a portion 4311 of a device, according to some embodiments of the invention.

In some embodiments, FIG. 43B shows an enlarged view of connection region 4311 of FIG. 43A.

FIG. 43C is a simplified schematic cross sectional view of a portion 4311 of a device, according to some embodiments of the invention.

In some embodiments, FIG. 43C illustrates a cross section of portion 4311 taken along line BB or taken along line CC of FIG. 43B.

Referring now to FIG. 43A, in some embodiments, device 4302 includes a body 4308 the shape of which includes one or more feature of body 4208 of FIGS. 42A-F (and/or body 1208 FIGS. 12A-H). In some embodiments, device 4302 includes a second supporting layer 4363 (also herein termed a “support”).

In some embodiments, support 4363 follows a shape of body 4308 for example, both body 4308 and support 4363 having a cup shape, or flattened cup shape and/or walls and a base. Where, in some embodiments, support 4363 is nested within base 4308 e.g. occupies a volume described by body 4308. In some embodiments, support 4363 follows a shape of body 4308 excluding a region 4371 of support 4363 adjacent to connection region 4311. Where, in some embodiments, within region 4371 support 4373 is separated from body 4308 e.g. by a distance corresponding to a height of connection region 4311 element/s. For example, where, in some embodiments, region 4371 length is at least about the same length as height 4373, or 0.5-4 times, or 1-3 times, or 1-2 times, or lower or higher or intermediate multiples or ranges of length 4373. In some embodiments, length 4371 is 1-20%, or 5-20%, or 5-10% of a width 4307 (in one or more dimension) of device 4302.

In some embodiments, portion/s of support 4363 following a shape of body 4308 have a separation between 4375 a contour of body 4308 and a contour of support 4363 which is at most 0.1-20%, or 0.5-10%, or 1-10% of device width 4307 and/or height 4358 of device.

In some embodiments, material of body 4308 is enters into connection region in a first direction and material of support 4373 enters into connection region in a second direction. Where the directions, in some embodiments, are generally opposite e.g. at about 180 degrees to each other.

In some embodiments, a separation between support 4373 and base 4308 being at least that of connection region rigid element/s at region 4371 and/or opposing directions of interaction of support 4373 and body 4308 with connection region 4311 contribute to a low profile of device 4302 when in a collapsed or crimped configuration e.g. as illustrated by device 4502 FIG. 45A.

In some embodiments, body 4308 is closed at connection region. In some embodiments, support 4363 is closed at connection region. For example, body 4308 and/or support 4363 each being constructed from a tubular material portion which is connected and/or closed at connection region 4311.

In some embodiments, closing of body 4308 and support 4363 is by separate connectors 4366, 4367 and bonding material portions 4351, 4355.

Referring now to FIG. 43B, in some embodiments, body 4308 is closed by bonding material 4351 and/or connector 4366 and/or holder 4306, the various options as described regarding connection of body 4008 by bonding material 4051 and/or connector 4066 and/or holder 4006 of FIGS. 40A-C.

In some embodiments, support 4363 is closed by bonding material 4353 and/or connector 4367 and/or holder 4306, the various options as described regarding connection of body 4008 by bonding material 4051 and/or connector 4066 and/or holder 4006 of FIGS. 40A-C.

In some embodiments, support 4363 and body 4308 are connected by connection of both portions to holder 4306. Alternatively or additionally, in some embodiments, support 4363 and body 4308 are connected by connecting connectors 4366, 4367 e.g. by welding material 4355. Alternatively or additionally, in some embodiments, support 4363 and body 4308 are connected by bonding material 4353, 4351 extending to connect the portions (e.g. as illustrated by bonding material 5051, FIGS. 50A-B). Alternatively or additionally, in some embodiments, function of connectors 4366, 4377 are provided by a single connector (e.g. as illustrated by connector 5066 FIGS. 50A-B).

In some embodiments, connector/s and/or bonding material region/s form closed shapes (e.g. are annular) around a central axis 4399 of device 4302 e.g. as illustrated in FIG. 43C.

Additional embodiments for attachment of body 4308 and support 4363 are illustrated in FIGS. 47A-C, FIGS. 48A-C, and FIGS. 49A-B.

In some embodiments, body 4308 and support 4363 are provided connected e.g. are constructed by a single folded tubular structure (e.g. tubular mesh). FIGS. 50A-B, in some embodiments, illustrate such an embodiment.

In some embodiments, body 4308 includes a recessed portion 4360 at a central region of body as body joins connection region 4311. Where, recessed portion 4360 includes one or more feature of recessed portion/s as described elsewhere in this document.

FIG. 44A is an image of a side view of a device 4402, according to some embodiments of the invention.

FIG. 44B is an image of a device 4102, according to some embodiments of the invention.

In some embodiments, FIGS. 44A-B illustrate device 4302 of FIGS. 43A-C.

Visible in FIGS. 44A-B are a body 4408, a support 4463, a central region 4457 of support 4463, a push-wire 4422, a connector 4466, a holder 4406. Visible in FIG. 44A-B at a region of body 4408 adjacent to connection region 4411, are wires protruding from body 4408. These wires, in some embodiments, are connected (e.g. laser welded) to body 4408. Where, for example, in some embodiments, a protruding wire is connected to a junction (e.g. by welding) where two wires of the mesh cross.

In some embodiments, holder 4406 includes electrically insulating material (e.g. PEEK) where, in some embodiments, push-wire 4422 is detached from device 4402 using electrical stimulation (e.g. as described elsewhere in this document) e.g. electrolytic detachment, e.g. electro-thermal detachment.

FIG. 45A is a simplified schematic cross sectional view of a device 4502 being delivered to a treatment region 4553, according to some embodiments of the invention.

FIG. 45B is a simplified schematic cross sectional view of a device 4502 being deployed in a treatment region 4553, according to some embodiments of the invention.

FIG. 45C is a simplified schematic cross sectional view of a deployed device 4502 in a treatment region 4553, according to some embodiments of the invention.

FIG. 45D is a simplified schematic cross sectional view of a deployed device 4502 in a treatment region 4553, according to some embodiments of the invention.

FIG. 45E is an image of a deployed device 4502 in a treatment region 4553, according to some embodiments of the invention.

In some embodiments, FIGS. 45A-E illustrate delivery, and deployment of the same device. In some embodiments, device 4502 includes one or more feature of device 4302 FIGS. 43A-C, and/or device 4402 FIGS. 44A-B.

Referring now to FIG. 45A, in some embodiments, a delivery catheter 4556 is positioned with an outlet of the catheter at the treatment region 4553 e.g. where the outlet is opposite an opening of an bifurcation aneurysm 4553. FIG. 45A, in some embodiments, illustrates device 4502 in a collapsed and/or crimped configuration. Where, in some embodiments, device 4502 is collapsed by folding both body 4508 and support 4363 in a same direction towards a central axis of device 4502 (central axis 4399 e.g. as illustrated in FIG. 43A). In some embodiments, a shape of support 4563 at a central region of device 4502 enables a smaller device cross sectional profile. For example, in some embodiments, narrowing of catheter 4556 with respect to device 4502 pushes body 4508 into contact with elements of connection region 4511, narrowing limited by size of connection region 4511 and thickness of body 4508 material. But, in some embodiments, where support 4563 curves away from connection region 4511, the profile of device 4508 is not additionally limited by thickness of support 4563 material.

In some embodiments, the device is advanced through catheter 4556 by pushing pressure applied to push-wire 4522.

Referring now to FIG. 45B, in some embodiments, as body 4508 (and optionally support 4556) of device 4502 exits catheter 4556 body 4508 (and optionally support 4556) elastically relaxes forming a curved closure (optionally, with support 4556, a double layer closure) to aneurysm 4553.

Where, FIG. 45C, in some embodiments, illustrates the device elastically relaxed into aneurysm 4553. In some embodiments, after device 4502 is deployed, push-wire 4522 is detached from device 4502 and withdrawn e.g. after and/or along with delivery catheter 4556. For example, leaving device 4502 in situ e.g. as illustrated in FIG. 45D. In some embodiments, holder 4506 is detached from device 4502 e.g. and withdrawn along with push-wire 4522. In some embodiments, holder 4506 is a portion of push-wire 4522.

FIG. 45E is an image of a deployed device 4502 in a treatment region 4553, according to some embodiments of the invention.

In some embodiments, FIG. 45E illustrates an x-ray image of deployed device 4502 within a bifurcation aneurysm 4553. Visible in FIG. 45E are device body 4508, and marker 4566. In FIG. 42E, delivery apparatus (e.g. including a push-wire and/or a catheter) has been withdrawn.

FIG. 46A is a simplified schematic cross sectional view of a device 4602 being delivered to a treatment region 4655, according to some embodiments of the invention.

FIG. 46B is a simplified schematic cross sectional view of a device 4602 being deployed in a treatment region 4655, according to some embodiments of the invention.

FIG. 46C is a simplified schematic cross sectional view of a deployed device 4602 in a treatment region 4655, according to some embodiments of the invention.

FIG. 46D is an image of a deployed device 4602 in a treatment region 4655, according to some embodiments of the invention.

In some embodiments, device 4602 includes one or more feature of device 4302 FIGS. 43A-C and/or of device 4403 FIGS. 44A-B and/or of device 4502 FIGS. 45A-E.

In some embodiments, device 4602 is delivered to a saccular aneurysm 4655 (and/or one or more other device described within this document) by bending of a delivery catheter 4656 (e.g. a distal end of the delivery catheter) so that an outlet of delivery catheter 4656 is directed towards an opening of aneurysm 4655. Where, in some embodiments, a distal portion of catheter is 4656 controllably bendable. For example, in some embodiments, a Bandit™ microcatheter is used. For example, in some embodiments, selective tension applied to an elongated element coupled to a distal end of the catheter is used to bend catheter 4656.

FIG. 52 is an image of a portion of a device 5208, according to some embodiments of the invention.

In some embodiments, the illustrated portion is an end region of a device (e.g. as defined hereinbelow).

In some embodiments, wire/s of mesh structure/s are connected e.g. at a distal end of structure/s of device bodies e.g. potentially preventing delamination of the mesh. For example, in some embodiments, distal ends of one or more tubular structures of one or more device as described in this document have connections between wires of the mesh. For example, open distal end 4391 of body 4308 and/or open distal end of support 4393 of the device of FIG. 43A. For example, open distal end 4091 of body 4008 of the device of FIGS. 40A-C).

In some embodiments, distal wire/s of device mesh are connected (e.g. by welding e.g. laser welding e.g. spot welding) of portion/s of the mesh together. Potentially, connection prevents delamination of the mesh.

In some embodiments, distal end/s 5299 of wires are connected. For example, where distal ends of two wires, at a distal intersection of the wires, is connected.

In some embodiments, each, or most e.g. more than 90%, of distal ends of wires of a device mesh are connected. In some embodiments, a portion of distal ends are connected, e.g. 20-90%, or lower or higher or intermediate percentages or ranges.

In some embodiments, a proportion of intersection/s proximal of the distal end are connected.

In some embodiments, interconnection of wires of the mesh is for a distal region of the device mesh. For example, interconnection being within a distal 0.1-3 mm, or 0.1-1 mm, or lower or higher or intermediate portions or ranges of the mesh e.g. as measured from a distal end of the mesh.

In some embodiments, a circumferential portion of a mesh body is interconnected e.g. a portion for which radial reinforcement is desired.

A potential advantage of increased connections between wires of the mesh is reduced likelihood of delamination and/or increased mechanical strength of the device e.g. at the region of connections. For example, increased expanding force (e.g. radial expanding force) e.g. of the device on walls of the aneurysm e.g. elastic expanding force of the device. For example, resistance (e.g. radial resistance) to crushing.

A potential disadvantage of fewer connections between wires of the mesh is prevention of movement of wires with respect to each other e.g. during expansion and/or contraction of the device.

FIG. 54 is an image of a portion of a device 5408, according to some embodiments of the invention.

In some embodiments, the illustrated portion is an end region of a device (e.g. as defined hereinbelow).

In some embodiments, one or more end (and/or end region) of one or more wire of a device mesh 5408 is blunt. A potential benefit being reduced likelihood of the wires traumatizing tissue walls e.g. penetration of the wire/s into a wall of an aneurysm in which the device is positioned. For example having a rounded shape and/or a larger cross section in one or more direction than proximal portion/s of the wire.

In an exemplary embodiment e.g. illustrated in FIG. 54, end/s of wires 5497 are welded to change the shape of the tip 5497 of the wire end e.g. by laser welding and/or by spot welding.

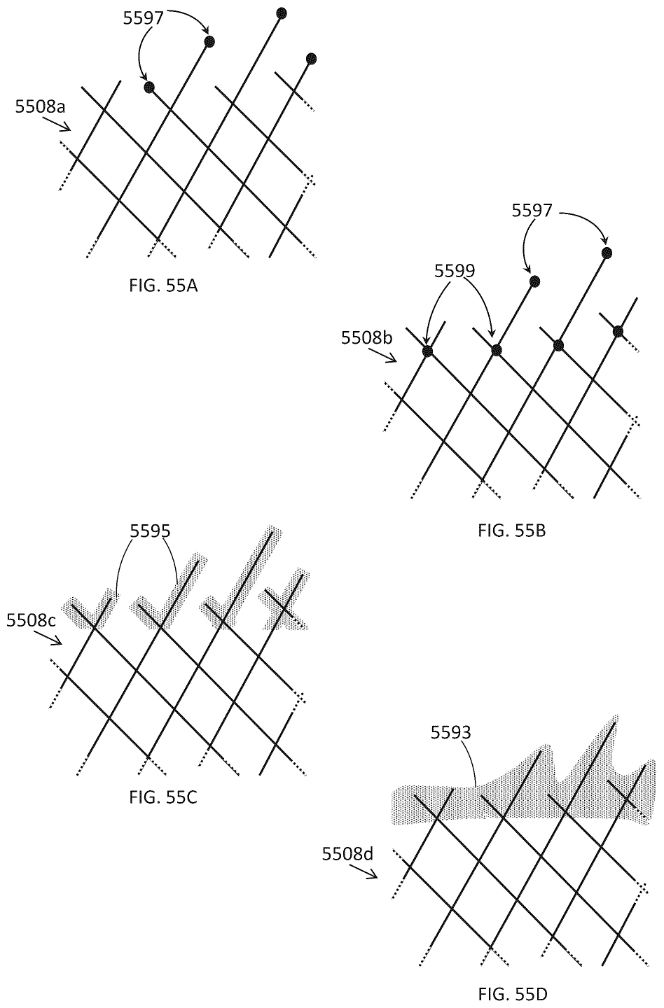

FIGS. 55A-D are simplified schematic device portions 5508a-d, according to some embodiments of the invention.

In some embodiments, the illustrated portions are an end regions of a device (e.g. as defined hereinbelow).

Referring now to FIG. 55A, in some embodiments, selected tips 5597 (e.g. less than all of the tips) of mesh 5508a are blunted (e.g. by welding).

Referring now to FIG. 55B, in some embodiments, tips 5597 are blunted and wires are connected 5599 e.g. connection including one or more feature as described regarding connections 5299 FIG. 52.

Referring now to FIG. 55C, in some embodiments, one or more end regions of device 5508c are blunted. For example, by coating e.g. dip coating of end/s of device 5508c.

Referring now to FIG. 55D, in some embodiments, one or more end regions of device 5508d are blunted by covering e.g. by a film and/or thin material layer 5593 connected to ends of device 5508d.

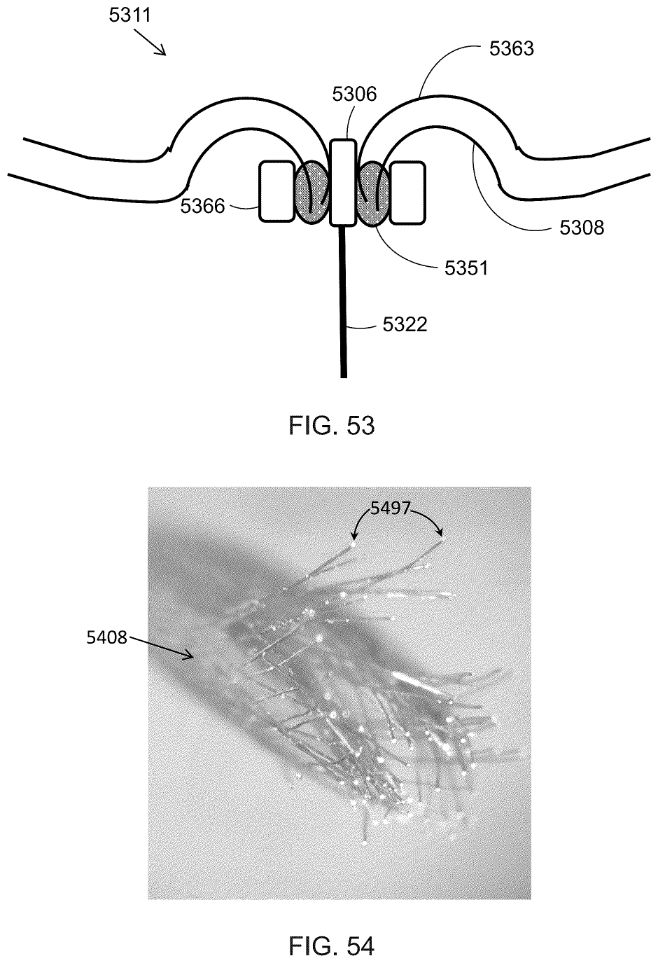

FIG. 53 is a simplified schematic cross sectional view of a portion 5311 of a device, according to some embodiments of the invention.

In some embodiments, a device includes one or more feature of the device depicted in FIGS. 43A-C, however with connection of a body 5308 (corresponding to body 4308 FIGS. 43A-C) and a support 5363 (corresponding to support 4363 FIGS. 43A-C) as illustrated in FIG. 53.

In some embodiments, proximal ends of a tubular structure forming body 4308 and proximal ends of a tubular structure forming support 4363 are both connected entering a proximal end of a connector 5366 lumen. Where connection options are, as described regarding FIGS. 43A-C e.g. bonding material only 5351, connector only 5366, both bonding material and connector.

In some embodiments, connection of body 5308 and support 5363 in the same direction enables the body and support to be in close proximity at the connection region, potentially increasing blocking of blood flow through the layers at the connection region.

In some embodiments, a free end of a portion of the device includes ends of mesh filaments where ends are not gathered by a connector. Where, in some embodiments, for a free end, the shape of a contour the ends is not deformed by one or more element. Where, in some embodiments, multiple ends (or not more than 5, or not more than 10, or not more than 20 or not more than 50 wire ends and/or end regions) and/or end region/s are not bent towards each other. In some embodiments, a free end portion of a device is not folded backwards on and/or into itself.

### Exemplary Connection Region Embodiments

In some embodiments, FIGS. 47A-C, FIGS. 48A-C, FIGS. 49A-B, FIGS. 50A-B illustrates embodiments of connection regions 4711, 4811, 4911, 5011 respectively suitable for use with body 4308 and/or support 4363 of device 4302 FIGS. 43A-C (e.g. to replace component/s of connection region 431 FIGS. 43A-C).

FIG. 47A is a simplified schematic cross sectional view of a portion 4711 of a device, according to some embodiments of the invention.

FIG. 47B is a simplified schematic cross sectional view of a portion of a device, according to some embodiments of the invention.

In some embodiments, FIG. 47B illustrates a cross section of portion 4711 taken along line DD of FIG. 47A.

FIG. 47C is a simplified schematic cross sectional view of a portion of a device, according to some embodiments of the invention.

In some embodiments, FIG. 47C illustrates a cross section of portion 4711 taken along line EE of FIG. 47A.

In some embodiments, body 4708 is attached to holder 4706 by bonding material 4753. In some embodiments, support 4763 is attached to holder 4706 by bonding material 4751. In some embodiments, connector 4766 is attached by bonding material 4751. In some embodiments, adhesive regions 4751, 4753 are attached to each other by weld 4755.

Referring to FIGS. 47B-C, in some embodiments, one or more of connector 4766, bonding material 4751, and bonding material 4753 have a closed shape around holder 4706 (e.g. annular shape).

FIG. 48A is a simplified schematic cross sectional view of a portion 4811 of a device, according to some embodiments of the invention.

FIG. 48B is a simplified schematic cross sectional view of a portion 4811 of a device, according to some embodiments of the invention.

In some embodiments, FIG. 48B illustrates a cross section of portion 4811 taken along line FF of FIG. 48A.

FIG. 48C is a simplified schematic cross sectional view of a portion 4811 of a device, according to some embodiments of the invention.

In some embodiments, FIG. 48C illustrates a cross section of portion 4811 taken along line GG of FIG. 48A.

Referring now to FIG. 48A, in some embodiments, body 4808 is attached between connectors 4866, 4867 e.g. by welding 4855. In some embodiments, body 4808 then bends outwards from connection region 4811 element/s and/or away from support 4863. In some embodiments, one or more of connectors 4866, 4867 are attached to holder 4806 by bonding material 4851, 4553 respectively.

Referring to FIGS. 48B-C, in some embodiments, one or more of connector 4866, connector 4867, bonding material 4851, and bonding material 4853 have a closed shape around holder 4806 (e.g. annular shape). In some embodiments weld 4855 has a closed shape around holder 4806 (but, in some embodiments, is not in contact with holder 4806) e.g. an annular shape.

FIG. 49A is a simplified schematic cross sectional view of a portion 4911 of a device, according to some embodiments of the invention.

FIG. 49B is a simplified schematic cross sectional view of a portion 4911 of a device, according to some embodiments of the invention.

In some embodiments, FIG. 49B illustrates a cross section of portion 4911 taken along line HH and/or line II of FIG. 49A.

FIG. 49A illustrates an embodiment including a single connector 4966 which connects both body 4908 and support 4963. In some embodiments, bonding material 4951 attaches connector 4966 to body 4908 and/or holder 4906. In some embodiments, bonding material 4953 attaches connector 4966 to support 4963 and/or holder 4906.

Referring to FIG. 49B, in some embodiments, one or more of connector 4966, bonding material 4951, and bonding material 4953 have a closed shape around holder 4906 (e.g. annular shape).

FIG. 50A is a simplified schematic cross sectional view of a portion of a device, according to some embodiments of the invention.

FIG. 50B is a simplified schematic cross sectional view of a portion of a device, according to some embodiments of the invention.

In some embodiments, FIG. 50B illustrates a cross section of portion 5011 taken along line JJ of FIG. 50A.

In some embodiments, body 5008 and support 5063 are formed by a single tubular material portion which is folded around connection region 5011. In some embodiments, a single connector 5066 and/or bonding material 5051 region attach body 5088 and support 5063 and/or attach body and support to holder 5006.

Referring to FIG. 50B, in some embodiments, one or more of connector 5066, and bonding material 5051 have a closed shape around holder 4906 (e.g. annular shape).

### General

It is expected that during the life of a patent maturing from this application many relevant devices for treatment of intravascular abnormality and/or aneurysm will be developed and the scope of the term intravascular device is intended to include all such new technologies a priori.

As used herein the term “about” refers to ±20%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An intravascular device comprising:
an expandable body having an expanded configuration and a contracted configuration, said expandable body in said expanded configuration having a convex semi-spherical curved shape having a continuous arch shaped cross section;
a support which provides a double layer to at least a portion of said expandable body
a connector;
wherein said connector is attached to said expandable body and to said support and recessed within the convex semi-spherical curved shape.

2. The device according to claim 1, wherein the intravascular device is set for expanding the expandable body prior to expansion of the support.

3. The device according to claim 1, wherein said expandable body joins said connector from a proximal end of said connector.

4. The device according to claim 1, wherein said support joins said connector from a distal end of said connector.

5. The device according to claim 1, wherein one or both of said expandable body and said support include mesh.

6. The device according to claim 1, wherein said expandable body, in said expanded configuration is sufficiently resilient to resist collapse within an aneurysm.

7. The device according to claim 1, wherein the semi-spherical curved shape having the continuous arch shaped cross section excludes a straight portion and/or a concave portion.

8. The device according to claim 1, wherein a distal end of the expandable body is open and a distal end of the support is open and non-connected to the open distal end of the expandable body.

9. The device according to claim 1, wherein the support has a convex semi-spherical curved shape having a continuous arch shaped cross section, and nested within a volume of the expandable body when in the expanded configuration.

10. The device according to claim 9, wherein the expandable body and the support are arranged as one of: concentric semi-spherical curved shapes, and parallel semi-spherical cured shapes.

11. The device according to claim 1, wherein the expandable body and the support are non-anchored at distal ends thereof when in the contracted configuration, and made of a flexible material selected for navigation through tortuous vasculature.

12. The device according to claim 1, wherein the connector includes radiopaque material.

13. The device according to claim 1, wherein the connector is configured for releasably connecting to a push wire.

14. The device according to claim 1, wherein distal ends of the support extend past distal ends of the expandable body.

15. The device according to claim 1, wherein the expandable body and the support each include 72-288 wires.

16. The device according to claim 1, wherein porosity of the intravascular device in the expanded configuration, by the expandable body and the support, is 60-75%.

17. The device according to claim 1, wherein said connector is attached to said expandable body and to said support and recessed within a center of the convex semi-spherical curved shape.

18. An intravascular device comprising:
an expandable body having an expanded configuration and a contracted configuration, said expandable body in said expanded configuration having a convex curved shape;
a support which provides a double layer to at least a portion of said expandable body
a connector,
wherein said expandable body joins said connector from a proximal end of said connector,
wherein said support joins said connector from a distal end of said connector; and
wherein said connector is attached to said expandable body and to said support and recessed within the convex curved shape.

19. An intravascular device comprising:
an expandable body having an expanded configuration and a contracted configuration, said expandable body in said expanded configuration having a convex curved shape;
a support which provides a double layer to at least a portion of said expandable body
a connector connected to the expandable body and to the support;
wherein said connector is attached to said expandable body and recessed within said convex curved shape,
wherein a distal end of the expandable body is open and a distal end of the support is open and non-connected to the open distal end of the expandable body.

20. An intravascular device comprising:
an expandable body having an expanded configuration and a contracted configuration, said expandable body in said expanded configuration having a convex semi-spherical curved shape;
a support which provides a double layer to at least a portion of said expandable body; and
a connector connected to the expandable body and to the support;
wherein the intravascular device is set for expanding the expandable body prior to expansion of the support.
